(19) United States
(12) Patent Application Publication
(10) Pub. No.: US 2004/0235054 A1
(43) Pub. Date: Nov. 25, 2004

(54) Novel encoding method for "one-bead one-compound" combinatorial libraries

(75) Inventors: Kit S. Lam, Davis, CA (US); Aimin Song, Davis, CA (US); Carlito B. Lebrilla, Davis, CA (US); Jinhua Zhang, Davis, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Regents of the University of California, Oakland, CA (US)

(21) Appl. No.: 10/811,331

(22) Filed: Mar. 25, 2004

Related U.S. Application Data
(60) Provisional application No. 60/458,470, filed on Mar. 28, 2003.

(51) Int. Cl. C12Q 1/68; G01N 33/53; G01N 33/543

(52) U.S. Cl. 435/7.1; 436/518

(57) ABSTRACT

The present invention provides a library of compounds, wherein each compound is encoded by several coding building blocks that are each separately attached to a solid support via a cleavable linker. Following screening of the compounds, the coding tags can be cleaved, and then characterized by mass spectrometry.

### CROSSED-REFERENCE TO RELATED APPLICATION

This application claims the benefit of U.S. Provisional Application Ser. No. 60/458,470, filed Mar. 28, 2003, the content of which is incorporated herein by reference.

### STATEMENT AS TO RIGHTS TO INVENTIONS MADE UNDER FEDERALLY SPONSORED RESEARCH AND DEVELOPMENT

A portion of the present invention was made under federally sponsored research and development under National Institutes of Health Grant No. CA 86364 and National Institutes of Health/National Cancer Institute Grant No. R33 CA 89706. The Government may have rights in certain aspects of this invention.

### BACKGROUND OF THE INVENTION

Combinatorial chemistry has become an essential component of the drug discovery process during the past decade. In 1991, the first “one-bead-one-compound” (OBOC) combinatorial library method was introduced (Lam, K. S. et al. Nature 1991, 354, 82-84). Using a “split-mix” synthesis procedure (Lam, K. S. et al. Nature 1991, 354, 82-84; Houghten, R. A. et al. Nature 1991, 354, 84-86; Furka, A. et al. Int. J. Peptide Protein Res. 1991, 37, 487-493), peptide or chemical libraries can be generated such that each bead display only one compound entity. With an on-bead screening assay, literally millions of compound-beads can be screened against specific molecular targets in a few days (Lam, K. S., et al. Nature 1991, 354, 82-84). Individual positive beads can then be isolated for structure determination. This approach has been successfully applied to the identification of ligands for a large number of biological targets (Lam, K. S., et al. Chem. Rev. 1997, 97, 411-448).

While peptide-beads can easily be microsequenced with Edman degradation using an automatic protein sequencer, structure determination of small molecule-beads is more challenging. Various indirect encoding methods have been developed to sequence small molecule-beads more readily, and the subject has been reviewed several times (Lam, K. S. et al. Chem. Rev. 1997, 97, 411-448; Czarnik, A. W. Curr. Opin. Chem. Biol. 1997, 1, 60-66; Xiao, X. Y. Front. Biotechnol. Pharm. 2000, 1, 114-149; Barnes, C., et al. Curr. Opin. Chem. Biol. 2000, 4, 346-350; Affleck, R. L. Curr. Opin. Chem. Biol. 2001, 5, 257-263). In most cases, a coding tag (comprising a coding building block and a coding linker) is synthesized on each bead in addition to the library component. These tags define the chemical history of any particular bead and hence the structure of the compound it supports. The coding tag is released from the bead following biological screening and analyzed by a highly sensitive analytical technique. For example, electron capture capillary gas chromatography has been successfully used for the detection of volatile halocarbon tags released from the beads via photolytic (Ohlmeyer, M. H. J., et al. Proc. Natl. Acad. Sci. USA 1993, 90, 10922-10926) or oxidative (Nestler, H. P., et al. J. Org. Chem. 1994, 59, 4723-4724) cleavage; reversed-phase HPLC with fluorescence detection has been used to determine the dansylated secondary amine tags released by mineral acid hydrolysis (Ni, Z. J., et al. J. Med. Chem. 1996, 39, 1601-1608; Murphy, M. M., et al. J. Am. Chem. Soc. 1995, 117, 7029-7030); and automatic microsequencers have been used to decode the peptide tags (Nikolaiev, V., et al. Peptide Res. 1993, 6, 161-170; Kerr, J. M., et al. J. Am. Chem. Soc. 1993, 115, 2529-2531; Liu, R., et al. J. Am. Chem. Soc. 2002, 124, 7678-7680).

Several physical encoding methods have also been described, but prove unattractive with huge diversity libraries (e.g. 250 000 compounds, Vaino, A. R.; Janda, K. D. Proc. Natl. Acad. Sci. 2000, 97, 7692-7696; Guiles, J. W., et al. Angew. Chem. Int. Ed. 1998, 37, 926-928), or cannot be enclosed in a single 80 μm bead (Nicolaou, K. C., et al. Angew. Chem., Int. Ed. Engl. 1995, 34, 2289-2291; Moran, E. J., et al. J. Am. Chem. Soc. 1995, 117, 10787-10788; Li, W., et al. J Comb. Chem. 2000, 2, 224-227).

Mass spectrometry (MS) has been widely used in the analysis of combinatorial libraries due to its intrinsic sensitivity, speed of analysis, specificity of detection and automation capability. Sequencing of peptides by MS is well-known (Biemann, K.; Martin, S. A. Mass Spectrom. Rev. 1987, 6, 1-76; Egner, B. J., et al. J. Org. Chem. 1995, 60, 2652-2653; Siuzdak, G.; Lewis, J. K. Biotechnol. Bioeng. 1998, 61, 127-134; Fitzgerald, M. C., et al. Bioorg. Med. Chem. Lett. 1996, 6, 979-982), and databases for MS sequencing of peptide libraries are commercially available. Because simultaneous cleavage and ionization occur under laser irradiation, peptides covalently attached to a single polymeric bead by a photosensitive linker can be directly sequenced by matrix-assisted laser desorption ionization (MALDI) MS (Siuzdak, G.; Lewis, J. K. Biotechnol. Bioeng. 1998, 61, 127-134; Fitzgerald, M. C., et al. Bioorg. Med. Chem. Lett. 1996, 6, 979-982). Structure-indicating fragments can be readily obtained by collision-induced dissociation (CID) or tandem MS (MS/MS).

The single bead analysis approach for peptides has also been applied to peptidomimetic and small molecule compound libraries (Haskins, N. J., et al. Rapid Commun. Mass Spectrom. 1995, 9, 1437-1440; Lorthioir, O., et al. Anal. Chem. 2001, 73, 963-970). For small libraries, the component on a single bead can be directly identified with the exact molecular mass and analysis of fragments. However, direct structure identification of a compound on a single bead isolated from a large diverse library of over 100 000 compounds will be very difficult if not impossible. This is because compounds from such combinatorial libraries often have molecular mass differences of less than 200 Da, and some of them are poorly- or non-ionizable.

Some indirect MS-based encoding/decoding strategies have also been described. For example, stable isotopes (¹³C or deuterium) have been incorporated into the coding building blocks to generate different isotopic patterns for structure characterization (Geysen, H. M., et al. Chem. Biol. 1996, 3, 679-688; Wagner, D. S., et al. Comb. Chem. High Throughput Screening 1998, 1, 143-153; Lane, S. J.; Pipe, A. Rapid Commun. Mass Spectrom. 2000, 14, 782-793).

Another elegant approach, but one limited to libraries of ionizable compounds with repeating subunits, such as peptides, has been developed by Youngquist et al. (Youngquist, R. S., et al. Rapid Commun. Mass Spectrom. 1994, 8, 77-81; Youngquist, R. S., et al. J. Am. Chem. Soc. 1995, 117, 3900-3906). In this approach, a small amount of a termination reagent is added at each synthetic step to generate a series of sequence-specific terminated products. Analysis of such products generated from one single bead, with MALDI-MS, enable one to reconstruct the complete peptide sequence.

Current chemical encoding methods have played an important role in the advancement of OBOC combinatorial chemistry. However, those methods often require orthogonal chemistries for tagging, and therefore additional synthetic steps. For example, in the halocarbon encoding method developed by Still et al. (Ohlmeyer, M. H. J., et al. Proc. Natl. Acad. Sci. USA 1993, 90, 10922-10926; Nestler, H. P., et al. J. Org. Chem. 1994, 59, 4723-4724), an additional 16-24 hours are needed to encode each building block. In addition to the increased time and cost, the tagging molecules themselves potentially could interfere with the binding of the target protein to the library compounds.

To address these problems, a new peptide-based encoding method has been developed that enables practitioners to topologically segregate the testing compounds from the coding tags (Liu, R., et al. J. Am. Chem. Soc. 2002, 124, 7678-7680). The resin beads are first derivatized with orthogonal protecting groups in the outer and inner regions separately. A coding tag precursor consisting of a sequence of α-amino acids, of which the side chains can be derivatized, is then constructed in the interior of the beads. During the library synthesis, building blocks are coupled to the outer scaffold and the side chains of the inner coding peptide simultaneously. In this way, the extra synthetic steps for coding the building blocks are eliminated by combining them with the library synthesis. After biological screening, the structures of active compounds can be easily determined by direct sequencing of the coding peptides with Edman degradation.

The utility and reliability of this encoding method has been confirmed. However, like other encoding strategies, this method has its limitations. First, this decoding method is based on Edman degradation, and therefore, is slow and expensive. Second, building blocks have to be carefully chosen to avoid retention time overlap of their amino acid derivatives during sequencing. Third, the choices for scaffolds are limited to those having the same functional groups as the side chains of commercially available trifunctional amino acids.

What is needed in the art is a highly efficient encoding strategy that is well-suited to libraries of small organic molecules. Surprisingly, the present invention meets this and other needs.

### SUMMARY OF THE INVENTION

The present invention relates to a novel method for encoding the building blocks of a compound during the synthesis of a compound library. The novel feature of this encoding method is the simultaneous preparation of a scaffold building block and a coding building block that is identical to or mimics the scaffold building block. In this manner, the preparation and encoding of a scaffold building block is carried in a single synthetic reaction. In addition, the coding building blocks are each individually attached to the solid support via a cleavable linker. Following preparation of the compound library, the coding building blocks are cleaved from the solid support and characterized to decode the compound.

In one aspect, the present invention provides a method for preparing a library of compounds, comprising: a) providing a plurality of individual synthesis templates each comprising a solid support, wherein the solid support has an interior portion and an exterior portion each with a plurality of reactive functional groups, wherein the solid support is linked to a scaffold via a scaffold linker, wherein the scaffold has at least two scaffold functional groups, and wherein at least two coding tag precursors, each comprising a coding functional group and a coding linker, are attached to the solid support; b) contacting a first synthesis template with a first reactive component such that a first scaffold functional group reacts with the first reactive component to afford a first scaffold building block, and a first coding functional group reacts with the first reactive component to afford a first coding building block; c) contacting the first synthesis template with a successive reactive component such that a subsequent scaffold functional group reacts with the successive reactive component to afford a subsequent scaffold building block, and a subsequent coding functional group reacts with the successive reactive component to afford a subsequent coding building block; d) repeating step c) until the first compound has been prepared; and e) subjecting additional synthesis templates to steps b)-d) with additional reactive components to prepare the library of compounds. In a preferred embodiment, the present invention provides a library of compounds prepared by the method above.

In another aspect, the present invention provides a method for preparing a library of compounds using a split-mix protocol, comprising: a) providing a population of individual synthesis templates each comprising a solid support, wherein the solid support has an interior portion and an exterior portion each with a plurality of reactive functional groups, wherein the solid support is linked to a scaffold via a scaffold linker, wherein the scaffold has at least two scaffold functional groups, and wherein at least two coding tag precursors, each comprising a coding functional group and a coding linker, are attached to the solid support; b) splitting the population of synthesis templates into two or more separate pools; c) contacting the population of synthesis templates with one or more first reactive components in the two or more separate pools such that a first scaffold functional group reacts with one of the first reactive components to afford a first scaffold building block, and a first coding functional group reacts with one of the first reactive components to afford a first coding building block, wherein the contacting step yields subsequent synthesis templates; d) mixing the subsequent synthesis templates from the two or more separate pools into a single pool; e) splitting the subsequent synthesis templates into two or more separate pools; f) contacting the subsequent synthesis templates in the two or more separate pools with a successive reactive component such that a subsequent scaffold functional group reacts with the successive reactive component to afford a subsequent scaffold building block, and a subsequent coding functional group reacts with the successive reactive component to afford a subsequent coding building block, wherein the contacting step yields further synthesis templates; and g) repeating steps d)-f), wherein the further synthesis templates of step f) become the subsequent synthesis templates of step d), until the library of compounds has been prepared.

### BRIEF DESCRIPTION OF THE DRAWINGS

FIG. 1. Schematic showing the stepwise preparation of a compound of a library, and concomitant encoding of the product of each reaction. Following preparation of the compound, the bead is screened for its biological activity. Those beads demonstrating activity have their coding building blocks cleaved and decoded via mass spectrometry.

FIG. 2. MALDI-FTMS spectrum of single-bead analysis for the resin containing library compound 11 and coding tags 12-14. This model compound was synthesized on beads with cleavable linker on both the outer layer and inner core.

FIG. 3. MALDI-FTMS decoding spectrum of compound 15 from the library of Example 5, screening against streptavidin.

FIG. 4. Comparison of MALDI-FIMS spectra of single-bead analysis for the resin containing library compound 1 and coding tags 2-4 of Example 6. a) Non-cleavable scaffold linker; b) Cleavable scaffold linker.

FIG. 5. A typical MALDI-FTMS decoding spectrum of streptavidin ligands from Example 6.

### DETAILED DESCRIPTION OF THE INVENTION

I. Definitions

As used herein, the term “library of compounds” refers to a collection of compounds on separate phase support particles in which each separate phase support particle contains a single structural species of the synthetic test compound. Each support contains many copies of the single structural species.

As used herein, the term “compound” refers to a small molecule consisting of 2 to 100, and more preferably, 2-20, functional groups, with or without a scaffold. In one embodiment, the compound is an aromatic heterocycle with three functional groups. In another embodiment, the compound can be a peptide or polymer.

As used herein, the terms “encode”, “encoded” and “encoding” refer to a library of compounds in which each distinct species of compound is paired on each separate solid phase support with at least one coding building block containing a functional group that is the same or mimics a particular functional group of the compound. In one embodiment, there is one coding building block for each functional group on the compound.

As used herein, the term “synthesis template” refers to a solid phase support with a scaffold and all coding functional groups individually attached to the scaffold. In one embodiment, the synthesis template is the starting point for preparing the library of compounds.

As used herein, the term “coding” is used as a prefix denoting that a particular feature or item is a part of the mechanism that encodes each functional group of the compounds in the library.

As used herein, the term “scaffold functional group” refers to a chemical moiety that is a precursor to the corresponding scaffold building block. Preferred scaffold functional groups include, but are not limited to, hydroxyl, carboxyl, amino, thiol, aldehyde, halogen, nitro, cyano, amido, urea, carbonate, carbamate, isocyanate, sulfone, sulfonate, sulfonamide, sulfoxide, amino acid, aryl, cycloalkyl, heterocyclyl, heteroaryl, etc. One of skill in the art will be aware of other common functional groups that are encompassed by the present invention.

As used herein, the term “coding functional group” refers to a chemical moiety that is a precursor to the corresponding coding building block. Preferred coding functional groups include, but are not limited to, hydroxyl, carboxyl, amino, thiol, aldehyde, halogen, nitro, cyano, amido, urea, carbonate, carbamate, isocyanate, sulfone, sulfonate, sulfonamide, sulfoxide, amino acid, aryl, cycloalkyl, heterocyclyl, heteroaryl, etc. One of skill in the art will be aware of other common functional groups that are encompassed by the present invention.

As used herein, the term “scaffold building block” refers to a chemical moiety that has been transformed by reacting a scaffold functional group with a reactive component.

As used herein, the term “coding building block” refers to a chemical moiety that has been transformed by reacting a coding functional group with a reactive component. The coding building block encodes the chemical functionality of the corresponding scaffold building block.

As used herein, the term “reactive component” refers to a chemical or reagant that is used to modify a functional group into a building block.

As used herein, the term “compound template” refers to a solid phase support with a scaffold and all coding building blocks individually linked to the solid phase support.

As used herein, the term “scaffold linker” refers to a chemical moiety that links the scaffold to the solid phase support. Scaffold linkers of the present invention, include, but are not limited to, aminobutyric acid, aminocaproic acid, 7-aminoheptanoic acid, 8-aminocaprylic acid, lysine, iminodiacetic acid, polyoxyethylene, glutamic acid, etc. In a further embodiment, linkers of the present invention can additionally comprise one or more β-alanines or other amino acids as spacers.

As used herein, the term “coding linker” refers to a chemical moiety that connects the coding functional group to the solid phase support. The coding linker also connects the coding building block to the solid phase support. The coding linkers of the present invention are cleavable, and comprise components that enhance the sensitivity of the analytical tools used for decoding. Coding linkers of the present invention, include, but are not limited to, aminobutyric acid, aminocaproic acid, 7-aminoheptanoic acid, 8-aminocaprylic acid, lysine, iminodiacetic acid, polyoxyethylene, glutamic acid, etc. In a further embodiment, linkers of the present invention can additionally comprise one or more)-alanines or other amino acids as spacers.

As used herein, the term “coding tag precursor” refers to a group that comprises a coding functional group and a coding linker.

As used herein, the term “coding tag” refers to a group that comprises a coding building block and a coding linker.

As used herein, the term “interior portion” refers to that portion of the solid phase support that substantially excludes the surface of the solid phase support.

As used herein, the term “exterior portion” refers to that portion of the solid phase support that substantially includes the surface of the solid phase support.

As used herein, the term “contacting” refers to the process of bringing into contact at least two distinct species such that they can react. In one embodiment, contacting an amine and an ester under appropriate conditions known to one of skill in the art would result in the formation of an amide.

As used herein, the term “coding sequence” refers to a set of coding building blocks that are separately attached to the solid support and encode the corresponding scaffold building blocks attached to the same solid support, or to a set of coding building blocks that are linked sequentially. In a preferred embodiment, coding sequence refers to a set of coding building blocks that are separately attached to the solid support and encode the corresponding scaffold building blocks attached to the same solid support.

As used herein, the term “mixing” refers to the act of combining individual elements such that they cannot be easily distinguished or separated.

II. General

As combinatorial chemistry has become an indispensable part of compound synthesis and drug discovery, the rapid and facile encoding and screening of the compounds generated is essential. While a variety of encoding methods have been developed in order to increase the speed and ease of encoding, they all have limitations when applied to large libraries of small molecules. The present invention provides a library of compounds attached to a separate phase support, preferably topologically segregated bifunctional resin beads. The compounds are prepared on the exterior of the beads while the coding building blocks are simultaneously prepared in the interior portion of the beads. Each functional group on the scaffold is encoded by an individual coding building block having the same chemical functionality as that on the scaffold. Following screening, the coding tags are cleaved from the positive beads and characterized by MS. The structures of active compounds can be readily identified according to the exact molecular masses of coding building blocks.

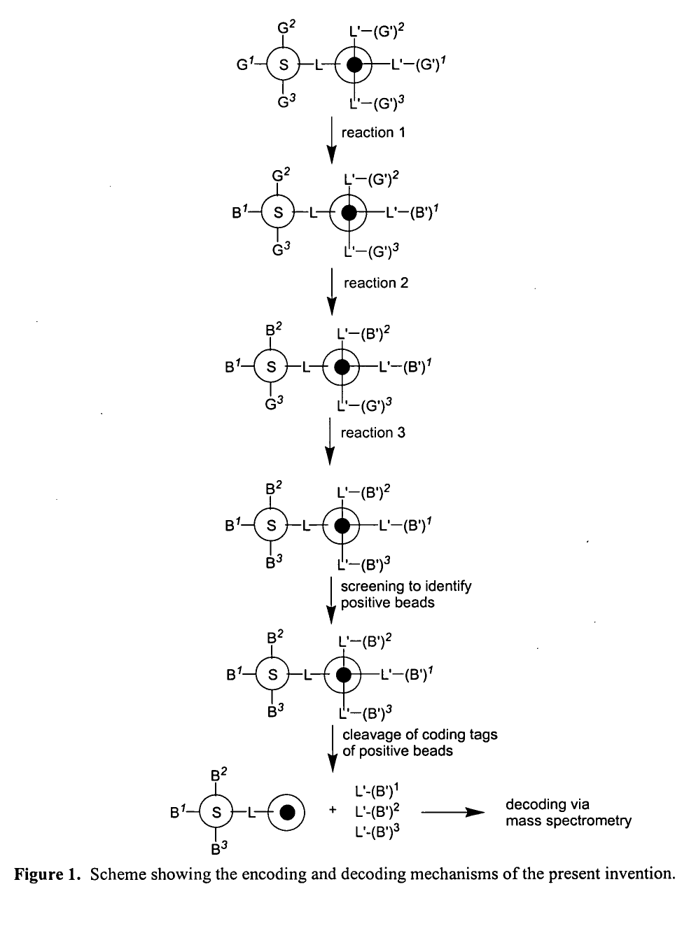

Using topologically segregated bifunctional resin beads, the library compounds are prepared on the exterior of the beads while the coding building blocks are simultaneously prepared in the interior (darkened) portion of the beads. FIG. 1 shows a synthesis template comprising a solid support attached to a scaffold (S) via a scaffold linker (L) on the exterior portion of the solid support. The scaffold has three scaffold functional groups (G¹, G² and G³), each unique from the others. In addition, the solid support has three coding tag precursors each separately attached to the solid support, and each comprising a coding functional group ((G′)¹, (G′)² or (G′)³) and a coding linker (L′). Each coding functional group is identical to, or mimics, one of the scaffold functional groups ((G′)¹ mimics G¹, (G′)² mimics G², etc.). As FIG. 1 demonstrates, each time the synthesis template is exposed to a particular reaction, one of the scaffold functional groups is converted to a scaffold building block (B¹, B² and B³), while at the same time, the corresponding coding functional group is converted to a coding building block ((B′)¹, (B′)² and (B′)³). Each scaffold building block is thereby encoded by an individual coding building block having the same chemical functionality ((B′)¹ mimics B¹, (B′)² mimics B², etc.). When the compound has been prepared, the bead is subjected to a screening method to determine its activity. After screening, the coding tags in the positive beads are released by chemical cleavage, and characterized by MS. The structures of active compounds can be readily identified according to the exact molecular masses of the coding tags.

III. Method for the Preparation of Encoded Compound Libraries

In one embodiment, the present invention provides a method for preparing a library of compounds, comprising: a) providing a plurality of individual synthesis templates each comprising a solid support, wherein the solid support has an interior portion and an exterior portion each with a plurality of reactive functional groups, wherein the solid support is linked to a scaffold via a scaffold linker, wherein the scaffold has at least two scaffold functional groups, and wherein at least two coding tag precursors, each comprising a coding functional group and a coding linker, are attached to the solid support; b) contacting a first synthesis template with a first reactive component such that a first scaffold functional group reacts with the first reactive component to afford a first scaffold building block, and a first coding functional group reacts with the first reactive component to afford a first coding building block; c) contacting the first synthesis template with a successive reactive component such that a subsequent scaffold functional group reacts with the successive reactive component to afford a subsequent scaffold building block, and a subsequent coding functional group reacts with the successive reactive component to afford a subsequent coding building block; d) repeating step c) until the first compound has been prepared; and e) subjecting additional synthesis templates to steps b)-d) with additional reactive components to prepare the library of compounds.

The libraries of compounds of the present invention are prepared using synthesis templates which are comprised of a solid support, preferably in the form of a bead, a scaffold having at least two scaffold functional groups, wherein the scaffold is attached to the solid support via a scaffold linker, and at least two coding tags, each comprising a coding functional group and a coding linker, and each separately attached to the solid support. One of skill in the art will recognize that other components may be incorporated.

Libraries of the present invention include libraries of compounds bound to a solid support, as well as libraries of compounds that are not bound to a solid support. In a preferred embodiment, the present invention provides a library of compounds bound to a solid support and prepared by the method described above. In another preferred embodiment, the method of the present invention further comprises the following step: f) cleaving each of the compounds from each of the synthesis templates. In yet another preferred embodiment, the present invention provides a library of compounds wherein the compounds are not bound to a solid support.

A. Encoding the Building Blocks of the Compound

The encoding strategy of the present invention utilizes cleavable coding functional groups in the interior of the solid support. In one embodiment, the coding functional groups of the present invention include, but are not limited to, hydroxyl, carboxyl, amino, thiol, aldehyde, halogen, nitro, cyano, amido, urea, carbonate, carbamate, isocyanate, sulfone, sulfonate, sulfonamide, sulfoxide, amino acid, aryl, cycloalkyl, heterocyclyl, heteroaryl, etc. Each of these coding functional groups is separately linked to the solid support through a coding linker, and contains a functional group that is identical to or mimics a corresponding scaffold functional group on the scaffold of the compound to be synthesized. In a preferred embodiment, the number of the coding functional groups is equal to the number of the scaffold functional groups.

The solid support of the present invention is first topologically derivatized (vide infra) with a protecting group on the outer layer using bi-phasic solvent approach (Liu et al. 2002). A cleavable linker, which can facilitate the mass determination of the coding building blocks, is then built into the interior of the bead. Coding functional groups are chosen according to the scaffold functional groups on the scaffold, and are coupled to the cleavable linker. Each coding functional group contains only one functional group, which has the same or similar chemical reactivity as the corresponding scaffold functional group on the scaffold. During the library synthesis, the reactive components couple to the outer scaffold functional groups and inner corresponding coding functional groups simultaneously.

The compounds of the present invention are prepared using a variety of synthetic reactions, including, but not limited to, amine acylation, reductive alkylation, aromatic reduction, aromatic acylation, aromatic cyclization, aryl-aryl coupling, [3+2] cycloaddition, Mitsunobu reaction, nucleophilic aromatic substitution, sulfonylation, aromatic halide displacement, Michael addition, Wittig reaction, Knoevenagel condensation, reductive amination, Heck reaction, Stille reaction, Suzuki reaction, Aldol condensation, Claisen condensation, amino acid coupling, amide bond formation, acetal formation, Diels-Alder reaction, [2+2] cycloaddition, enamine formation, esterification, Friedel Crafts reaction, glycosylation, Grignard reaction, Homer-Emmons reaction, hydrolysis, imine formation, metathesis reaction, nucleophilic substitution, oxidation, Pictet-Spengler reaction, Sonogashira reaction, thiazolidine formation, thiourea formation and urea formation. The reactive components of the present invention are those that enable the reactions above to occur. These include, but are not limited to, nucleophiles, electrophiles, acylating agents, aldehydes, carboxylic acids, alcohols, nitro, amino, carboxyl, aryl, heteroaryl, heterocyclyl, boronic acids, phosphorous ylides, etc. In order to encode each scaffold building block, the corresponding coding building block can be simultaneously prepared by a coding reaction that encodes the functionality of the corresponding scaffold building block. One of skill in the art can envision other synthetic reactions and reactive components useful in the present invention. Table 1 highlights several reactions that can be used to prepare the compounds of the present invention, and the corresponding coding reactions and reactive components. In Table 1, one of skill in the art will understand that radicals R, R¹ and R² can be, for example, hydrogen, alkyl, cycloalkyl, heterocyclyl, aryl and heteroaryl, all optionally substituted. One of skill in the art will further understand that radical Ar is an aryl, which can be, for example, phenyl, naphthyl, pyridyl and thienyl. In addition, one of skill in the art will understand that radical X can be, for example, hydrogen, halogen alkyl, cycloalkyl, heterocyclyl, aryl and heteroaryl.

TABLE 1
Proposed coding strategy for 15 coupling reactions.

| Reaction | Reaction scheme | | Reference | Proposed coding reaction | |
|---|---|---|---|---|---|
| Amine acylation | 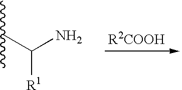 | 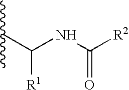 | Perumattam et al. 1998 | 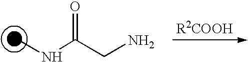 | 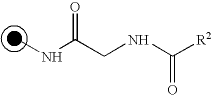 |
| Reductive alkylation | 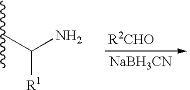 | 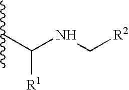 | Gordon and Steele 1995 | 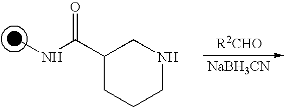 | 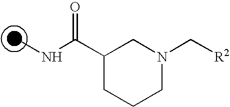 |
| Aromatic reduction, acylation and cyclization | 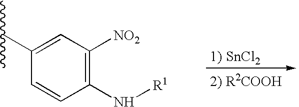 | 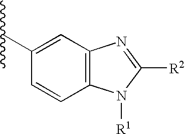 | Mazurov 2000 | 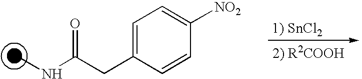 | 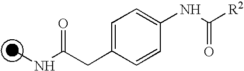 |
| Aryl-Aryl coupling | 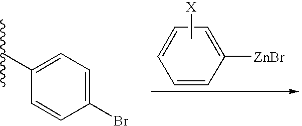 | 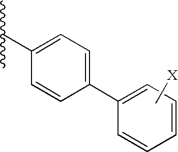 | Marquais and Arlt 1996 | 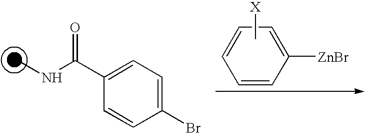 | 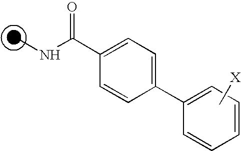 |
| [3 + 2]Cyclo- addition | 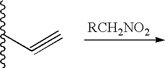 | 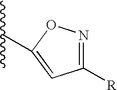 | Park and Kurth 1999 | 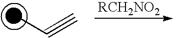 | 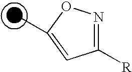 |
| Mitsunobu reaction | 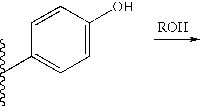 | 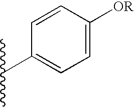 | Gentles et al. 2002 | 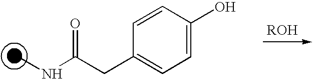 | 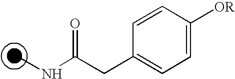 |
| Nucleo- philic aromatic substitu- tion | 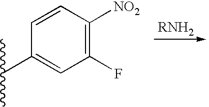 | 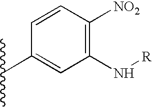 | Wei and Phillips 1998 | 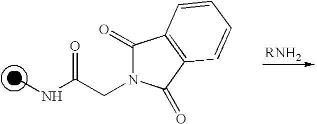 | 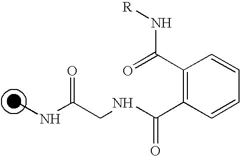 |
| Michael addition | 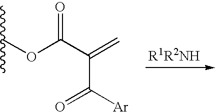 | 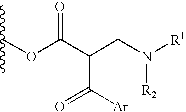 | Garibay et al. 1998 | 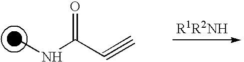 | 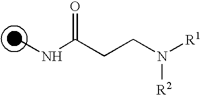 |
| Wittig reaction | 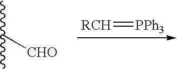 | 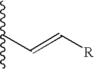 | Veerman et al. 1998 | 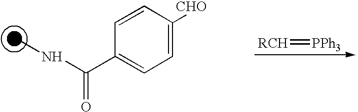 | 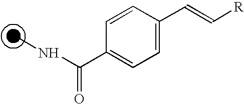 |
| Knoeve- nagel conden- sation | 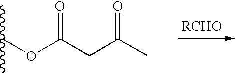 | 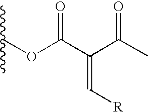 | Gordeev et al. 1996 | 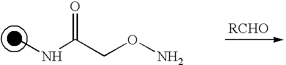 | 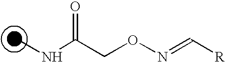 |
| Reductive amination | 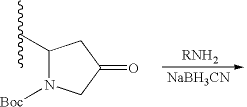 | 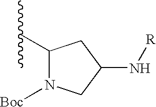 | Bray et al. 1995 | 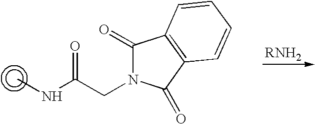 | 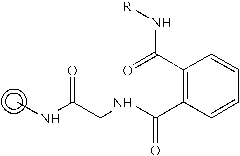 |
| Heck reaction | 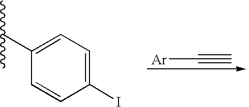 | 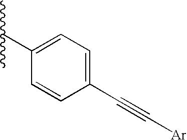 | Yu et al. 1994 | 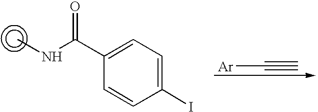 | 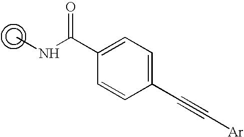 |
| Stille reaction | 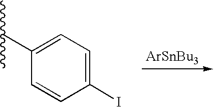 | 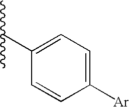 | Forman and Sucholeiki 1995 | 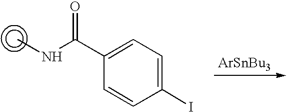 | 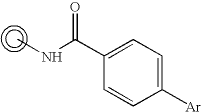 |
| Suzuki reaction | 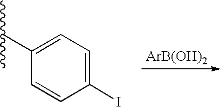 | 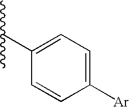 | Frenette and Friesen 1994 | 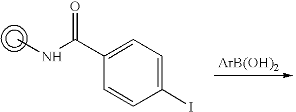 | 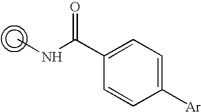 |
| Aldol conden- sation | 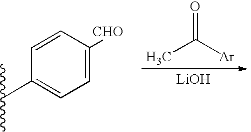 | 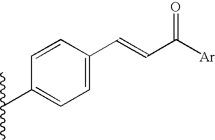 | Marzinzik and Felder 1998 | 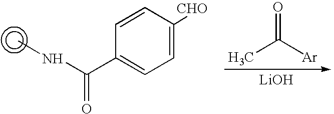 | 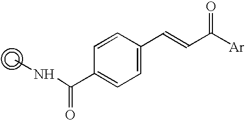 |
| Claisen conden- sation | 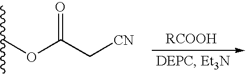 | 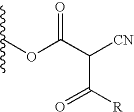 | Sim et al. 1998 | 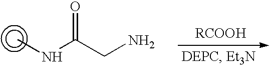 | 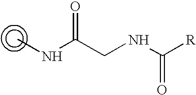 |

Contacting the scaffold functional group with a reactive component results in conversion of the scaffold functional group to the scaffold building block. In a similar manner, contacting the coding functional group with another of the same reactive component results in conversion of the corresponding coding functional group to the appropriate coding building block. In this manner, the scaffold building block is encoded by a coding building block. It would be apparent to one of skill in the art that “contacting” one component with another means to bring them into such close proximity that they can react with one another to afford a third component, the product.

In another embodiment of the present invention, the reactive component reacts with the scaffold functional group and the coding functional group via a reaction selected from the group consisting of amine acylation, reductive alkylation, aromatic reduction, aromatic acylation, aromatic cyclization, aryl-aryl coupling, [3+2] cycloaddition, Mitsunobu reaction, nucleophilic aromatic substitution, sulfonylation, aromatic halide displacement, Michael addition, Wittig reaction, Knoevenagel condensation, reductive amination, Heck reaction, Stille reaction, Suzuki reaction, Aldol condensation, Claisen condensation, amino acid coupling, amide bond formation, acetal formation, Diels-Alder reaction, [2+2] cycloaddition, enamine formation, esterification, Friedel Crafts reaction, glycosylation, Grignard reaction, Homer-Emmons reaction, hydrolysis, imine formation, metathesis reaction, nucleophilic substitution, oxidation, Pictet-Spengler reaction, Sonogashira reaction, thiazolidine formation, thiourea formation and urea formation. Other reactions useful in the present invention will be apparent to one of skill in the art.

In a preferred embodiment, the compounds of the library are prepared in parallel. In this embodiment, the compounds of the library can be prepared either using the split-mix methodology or in multi-partition containers. One of skill in the art will appreciate that other methods of preparing the compounds of the library in a parallel fashion are useful.

In yet another embodiment, at least one of the synthesis templates has a structure of formula I:

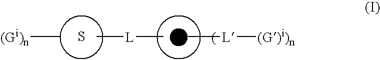

wherein (Gⁱ)ₙ represents n independent scaffold functional groups, G^{l} to Gⁿ, wherein each Gⁱ is one of the scaffold functional groups;

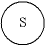

is the scaffold; L is the scaffold linker;

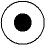

is the solid support, wherein the darkened portion represents the interior portion of the solid support, and the lightened portion represents the exterior portion of the solid support; (-L′-(G′)ⁱ)ₙ represents n independent coding tag precursors, wherein each of the coding tag precursors comprises one of n independent coding functional groups, (G′)^{l} to (G′)ⁿ, each linked to the solid support via one of n coding linkers, wherein each (G′)ⁱ is one of the coding functional groups, and L′ is the coding linker; subscript n is an integer from 2 to 10; and superscript i is an integer from 1 to n. In Formula I, the scaffold is linked to the solid support through a scaffold linker, L. Attached to the scaffold are at least two scaffold functional groups, Gⁱ. Also attached to the solid support are several separately attached coding tag precursors, each comprising a coding functional group, (G′)ⁱ, and a coding linker, L′.

In a further embodiment, the synthesis template has a structure of formula Ia:

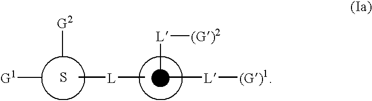

In Formula Ia, n=2, resulting in two scaffold functional groups (G¹ and G²) and two coding functional groups ((G′)¹ and (G′)²). Formula Ia demonstrates the coding functional groups as separately attached to the solid support.

In another embodiment, the synthesis template has a structure of formula Ib:

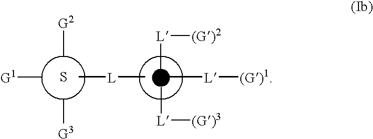

In Formula Ib, n=3, resulting three scaffold functional groups (G¹, G² and G³) and three corresponding coding functional groups ((G′)¹, (G′)² and (G′)³), each separately attached to the solid support.

In a preferred embodiment, the synthesis template has a structure of formula Ic:

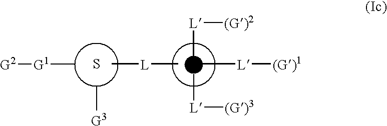

wherein each of G¹, G² and G³ is one of the scaffold functional groups;

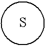

is the scaffold;

L is the scaffold linker;

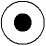

is the solid support, wherein the darkened portion represents the interior portion of the solid support, and the lightened portion represents the exterior portion of the solid support; and each of -L′-(G′)¹, -L′-(G′)² and -L′-(G′)³ is one of the coding tag precursors, each comprising a coding functional group linked to the solid support via a coding linker. In Formula Ic, n=3 as in Formula Ib. While there are three scaffold functional groups, one (G²) is linked to the scaffold through another scaffold functional group (G¹). Conversion of the G¹ scaffold functional group to the corresponding scaffold building block does not interfere with its linking G² to the scaffold. Likewise, conversion of G² to the corresponding scaffold building block does not interfere with G¹. Also attached to the solid support are three separately attached coding tag precursors.

In another preferred embodiment, the method of the present invention provides a library of compounds wherein at least one of the synthesis templates has a structure of formula II:

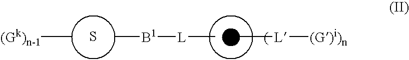

wherein B¹ represents a first scaffold building block; (G^{k})ₙ₋₁ represents n−1 independent scaffold functional groups, G² to Gⁿ, wherein each G^{k} is one of said scaffold functional groups;

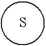

is the scaffold; L is the scaffold linker;

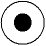

is the solid support, wherein the darkened portion represents the interior portion of the solid support, and the lightened portion represents the exterior portion of the solid support; (-L′-(G′)ⁱ)ₙ represents n independent coding tag precursors, wherein each of the coding tag precursors comprises one of n independent coding functional groups, (G′)^{l} to (G′)ⁿ, each linked to the solid support via one of n coding linkers, wherein each (G′)ⁱ is one of the coding functional groups, and L′ is the coding linker; subscript n is an integer from 2 to 10; superscript i is an integer from 1 to n; and superscript k is an integer from 2 to n. In Formula II, the scaffold is linked to the solid support through a scaffold linker. Attached to the scaffold are at least two scaffold functional groups, and one pre-attached scaffold building block linking the scaffold to the scaffold linker. Also attached to the solid support are several coding tag precursors, each separately linked to the solid support member.

In a further preferred embodiment, the synthesis template has a structure of formula IIa:

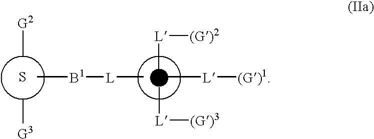

In Formula IIa, n=3, with three scaffold functional groups (G¹, G² and G³) wherein one (G^{l}) links the scaffold to the scaffold linker. In addition, the three coding functional groups ((G′)¹, (G′)² and (G′)³) are each separately attached to the solid support.

In a preferred embodiment, the method of the present invention provides a library of compounds wherein the steps a)-d) afford a compound template of formula III:

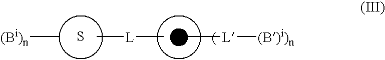

wherein (Bⁱ)ₙ represents n independent scaffold building blocks, B^{l} to Bⁿ, wherein each Bⁱ is one of the scaffold building blocks;

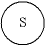

is the scaffold; L is the scaffold linker;

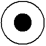

is the solid support, wherein the darkened portion represents the interior portion of the solid support, and the lightened portion represents the exterior portion of the solid support; (-L′-(B′)ⁱ)ₙ represents n independent coding tags, wherein each of the coding tags comprises one of n independent coding building blocks, (B′)^{l} to (B′)ⁿ, each linked to the solid support via one of n coding linkers, wherein each (B′)ⁱ is one of the coding building blocks, and L′ is the coding linker; subscript n is an integer from 2 to 10; and superscript i is an integer from 1 to n. Formula III represents the product formed following the method of the present invention for the preparation of a library of compounds. In Formula III, the scaffold is linked to the solid support through a scaffold linker. Attached to the scaffold are the scaffold building blocks. Also attached to the solid support are several separately attached coding tags. Each coding tag comprises one coding building block linked to the solid support member through a coding linker. One of skill in the art can envision other configurations for attachment of the scaffold functional groups to the scaffold that are useful in the present invention.

The compound templates of Formula III are prepared following the method of the present invention comprising: a) providing a plurality of individual synthesis templates according to formula Ib; b) contacting a first synthesis template with a first reactive component to afford the following structure:

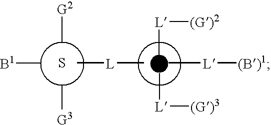

c) contacting the first synthesis template with a successive reactive component to afford the following structure:

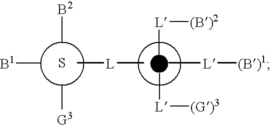

d) repeating step c) to prepare the compound attached to the following compound template according to the description above:

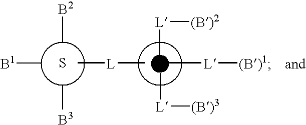

e) subjecting additional of the synthesis templates to steps b)-d) with additional of the reactive components in order to prepare the library of compounds. As demonstrated here, the corresponding scaffold functional group and coding functional group are simultaneously converted to the scaffold building block and coding building block, respectively. Each reaction step converts a scaffold functional group and a coding functional group to a scaffold building block and a coding building block, respectively. The process continues until all the scaffold functional groups have been converted to scaffold building blocks, and all the coding functional groups have been converted to coding building blocks. One of skill in the art will recognize that additional steps may be introduced into the method without detracting from the method.

In another embodiment of the present invention, the method of preparing the compound templates of Formula III further comprises the following step: f) cleaving each of the compounds from each of the compound templates.

In a preferred embodiment, on each of the synthesis templates, each of the scaffold building blocks is encoded by a single coding building block.

B. Decoding the Library

In one embodiment of the present invention, the method of the present invention further comprises the following step: f) decoding each of the compounds by cleaving each of the coding tags from the synthesis template and analyzing the coding tags to determine the identity of the corresponding scaffold building blocks. In a preferred embodiment, the analyzing is carried out via mass spectrometry. One of skill in the art can envision other analytical tools that are useful in the present invention.

Decoding is accomplished by cleaving all the coding tags at once and analyzing the releasates by mass spectrometry. In a preferred embodiment, matrix-assisted laser desorption/ionization Fourier transform mass spectrometry (MALDI-FTMS) is used due to its high mass resolution, accuracy and sensitivity. A hydrophilic linker (-linker-Phe-Phe-Met-) that links the coding building blocks with the solid support (resin bead) is designed to facilitate mass spectrometry analysis. Methionine is stable to many chemical reactions, but it can be readily cleaved by cyanogen bromide (CNBr). Its cleavage is very reliable and specific, and offers clean products, which are suitable to single-bead analysis. Two phenylalanines are introduced into the linker to increase the molecular weight of the final cleavage products, so that their signals can be easily distinguished from those of matrix and impurities. An additional hydrophilic linker is selected to enhance the solubility of the final cleaved products in the extraction solvent (50% acetonitrile/water). The whole linker has excellent chemical stability, and is very suitable for MALDI-FTMS detection.

Using this method, it is possible to detect several coding tags in the inner core (40% substitution in total) of a single bead. Because only the molecular mass of the coding tags is needed to identify the structure of library compound, a very small amount of a coding tag is sufficient for MALDI-FTMS detection. Considering a library based on a scaffold with four diversities, if 100 different reactive components are used in each synthetic step, a library containing 1004=100,000,000 compounds will be generated, while the total number of coding tag structures required is only 400. Because of the high precision and sensitivity of MALDI-FTMS, it is not difficult to accurately identify each of the 400 different building blocks used in the library synthesis. Since each coding functional group has only one functional group, the chemical structure of the final coding building blocks is very simple. Furthermore, all the coding tags are located in the interior of the bead, and each of them constitutes only about 10% equivalent of the whole bead, it is anticipated that this encoding method will have minimal effect on biological screening.

C. Solid Supports

A separate phase support suitable for use in the present invention is characterized by the following properties: (1) insolubility in liquid phases used for synthesis or screening; (2) capable of mobility in three dimensions independent of all other supports; (3) containing many copies of each of the synthetic test compound and, if present, the coding sequence attached to the support; (4) compatibility with screening assay conditions; and (5) being inert to the reaction conditions for synthesis of a test compound. A preferred support also has reactive functional groups, including, but not limited to, hydroxyl, carboxyl, amino, thiol, aldehyde, halogen, nitro, cyano, amido, urea, carbonate, carbamate, isocyanate, sulfone, sulfonate, sulfonamide, sulfoxide, etc., for attaching a subunit which is a precursor to each of the synthetic test compound and coding building blocks, or for attaching a linker which contains one or more reactive groups for the attachment of the monomer or other subunit precursor.

As used herein, separate phase support is not limited to a specific type of support. Rather a large number of supports are available and are known to one of ordinary skill in the art. In a preferred aspect, the separate phase support is a solid phase support, although the present invention encompasses the use of semi-solids, such as aerogels and hydrogels. Solid phase supports include silica gels, resins, derivatized plastic films, glass beads, cotton, plastic beads, alumina gels, polysaccharides such as Sepharose and the like, etc. A suitable solid phase support can be selected on the basis of desired end use and suitability for various synthetic protocols. For example, in polyamide synthesis, useful solid phase support can be resins such as polystyrene (e.g., PAM-resin obtained from Bachem Inc., Peninsula Laboratories, etc.), POLYHIP™ resin (obtained from Aminotech, Canada), polyamide resin (obtained from Peninsula Laboratories), polystyrene resin grafted with polyethylene glycol (TentaGel™, Rapp Polymere, Tubingen, Germany), polydimethyl-acrylamide resin (available from Milligen/Biosearch, California), or PEGA beads (obtained from Polymer Laboratories). Preferred solid phase synthesis supports for specific syntheses are described below. Thus, each resin bead is functionalized to contain both synthetic test compound and the corresponding coding structures. In a variation of this approach, the synthetic test compound and coding building blocks are attached to the solid support through linkers such as those described below. One of skill in the art will recognize that while many types of solid supports are useful in the present invention, topologically segregated solid supports are particularly useful.

Topology of Solid Supports

A variety of approaches for topologically separating the synthetic test compound and coding tags on a solid support in order to generate libraries are useful.

Topologically separating the synthetic test compound and the coding tag refers to the separation in space on a support. For example, if the support is a resin bead, separation can be between the surface and the interior of the resin bead of a significant number of the ligand-candidate molecules from a significant number of the coding tags. Preferably, the surface of the support contains primarily synthetic test compound molecules and very few coding tags. More preferably, the surface of the support contains greater than 90% synthetic test compound and less than 10% coding tags. Even more preferably, the surface of the support contains greater than 99% synthetic test compound molecules and less than 1% coding tags; most preferably, it contains more than 99.9% synthetic test compound and less than 0.1% coding tags. The advantage of such an arrangement is that interference of the coding tag in a binding screening assay is limited. It is not necessary that the topological area that contains the coding tag, i.e., the interior of a resin bead, be free of the synthetic test compound.

In one embodiment of the present invention, it is useful to have a high concentration of compounds attached to the exterior portion of the beads. This is advantageous when large quantities of a compound are desired. In a preferred embodiment, it is useful for at least 50% of the reactive functional groups on the exterior portion of the solid support to be linked to a compound. In a more preferred embodiment, it is useful for at least 75% of the reactive functional groups on the exterior portion of the solid support to be linked to a compound. In a most preferred embodiment, it is useful for at least 90% of the reactive functional groups on the exterior portion of the solid support to be linked to a compound.

In another embodiment, the steric and binding requirements for on-bead screening dictate that there be only a few compounds attached to the exterior portion of the solid support. In a preferred embodiment, it is useful for less than 50% of the reactive functional groups on the exterior portion of the solid support to be linked to a compound. In a more preferred embodiment, it is useful for less than 25% of the reactive functional groups on the exterior portion of the solid support to be linked to a compound. In a most preferred embodiment, it is useful for less than 10% of the reactive functional groups on the exterior portion of the solid support to be linked to a compound.

As discussed above, the coding tags are optionally segregated in the interior of the support particle. However, coding tags can also be segregated to the surface of a support particle, or to one side of a support particle.

One general approach for the topological separation of synthetic test compound from coding tags involves the selective derivatization of reactive sites on the support based on the differential accessibility of the coupling sites to reagents and solvents. For example, regions of low accessibility in a resin bead are the interior of the bead, e.g., various channels and other cavities. The surface of a resin bead, which is in contact with the molecules of the solution in which the bead is suspended, is a region of relatively high accessibility. Methods for effecting the selective linkage of coding functional groups and scaffolds to a suitable solid phase support include, but are not limited to, the following.

(i) Selective Derivatization Of Solid Support Surfaces Via Controlled Photolysis

Two approaches can be used. In one, a functionalized solid support is protected with a photocleavable protecting group, e.g., nitroveratryloxycarbonyl (Nvoc) (Patchornik et al. J. Am. Chem. Soc. 1970, 92, 6333). The Nvoc-derivatized support particles are arranged in a monolayer formation on a suitable surface. The monolayer is photolyzed using light of controlled intensity so that the area of the bead most likely to be deprotected by light will be the area of the bead in most direct contact with the light, i.e., the exterior surface of the bead. The resulting partially deprotected beads are washed thoroughly and reacted with a scaffold containing a light-stable protecting group. Following the reaction with the scaffold, the beads are subjected to quantitative photolysis to remove the remaining light-sensitive protecting groups, thus exposing functional groups in less light-accessible environments, e.g., the interior of a resin bead. After this quantitative photolysis, the support particles are further derivatized with an orthogonally-protected coding functional group, e.g., Fmoc-protected amino acid. The resulting solid support bead will ultimately contain synthetic test compound segregated primarily on the exterior surface and coding tags located in the interior of the solid phase support bead (see FIG. 1).

An alternative photolytic technique for segregating coding building blocks and synthetic test compound on a support involves derivatizing the support with a branched linker, one branch of which is photocleavable, and attaching the coding functional groups to the photosensitive branch of the linker. After completion of the synthesis, the support beads are arranged in a monolayer formation and photolyzed as described above. This photolysis provides beads which contain patches of synthetic test compound for selective screening with minimal interference from the coding building blocks.

(ii) Selective Derivatization of Solid Support Surfaces Using Chemical or Biochemical Approaches

The efficacy of these chemical and biochemical derivatizations depends on the ability of exterior surface functional groups, which are exposed, to react faster than other groups in the interior which are not exposed. It has been observed, for example, that antibodies cannot bind to peptide ligands in the interior of a non-porous resin solid phase support. Therefore, using differences in steric hindrance imposed by the structure of the support or by modulating the swelling of a bead through choice of reaction solvent, reactive groups on the exterior of the bead that are accessible to macromolecules or certain reagents can be reacted selectively relative to reactive groups in the interior of the bead. Therefore, the reactive groups in the exterior of the bead can be modified for the synthesis of the synthetic test compound, while interior reactive groups can be modified for preparation of the coding tags, or both the coding tags and synthetic test compound. Since the number of reactive groups inside a resin bead is much larger than the number of groups on the outer surface, the actual number of coding tags will be very large, providing enough coding tags for accurate mass spectral analysis, and thus the decoding of the structure of the synthetic test compound. A variety of chemical and biochemical approaches are contemplated including the following:

(a) Use of Polymeric Deprotecting Agents to Selectively Deprotect Parts of the Exterior of a Solid Support Bead Carrying Protected Functional Groups

The deprotected functional groups are used as anchors for the scaffold. The functional groups which remain protected are subsequently deprotected using a nonpolymeric deprotecting agent and used as anchors for the attachment of the coding functional groups. In a specific embodiment, this method involves use of enzymes to selectively activate groups located on the exterior of beads which have been derivatized with a suitable enzyme substrate. Due to their size, enzymes are excluded from the interior of the bead. In an example, infra, an enzyme completely removes a substrate from the surface of a resin bead, without significantly affecting the total amount of substrate attached to the bead, i.e., the interior of the bead. The removal of substrate exposes, and thus activates, a reactive site on the bead. The enzyme-modified groups of the solid support are used to anchor the scaffold and those groups that escaped modification are used to anchor the majority of the coding functional groups.

(b) Use of a Polymeric Protecting Group to Selectively Block Exposed Unprotected Functional Groups On the Exterior of a Support Bead

The unprotected functional groups in the interior of the support are used to anchor the coding functional groups. The remaining protected functional groups are then deprotected and used as anchors for the scaffolds of the library.

(c) Creating a Different State In The Interior of the Bead

Through the judicious selection of solvents, it is possible to swell the beads with one solvent, which is subsequently frozen, and then add the beads to a second solvent at a low temperature. For example, by freezing water inside the beads, then reacting the beads in an organic solvent at low temperature, the water in the interior of the bead remains frozen. Thus the surface of the bead, but not the interior, can be selectively reacted.

(d) Use of a Biphasic Solvent Environment

In a similar fashion to method (c) above, the beads are first swelled with an aqueous solvent, followed by derivatization of the beads in an appropriate organic solvent such that the water in the interior of the bead remains there. In this manner, only the functional groups on the outside of the bead (those not in the aqueous solvent) are derivatized (Liu, R. et al. J of the Am. Chem. Soc. 2002, 124, 7678).

D. Linkers

The solid supports of the present invention can also comprise linkers or an arrangement of linkers. As used herein, a linker refers to any molecule containing a chain of atoms, e.g., carbon, nitrogen, oxygen, sulfur, etc., that serves to link the molecules to be synthesized on the solid support with the solid support. The linker is usually attached to the support via a covalent bond, before synthesis on the support starts, and provides one or more sites for attachment of precursors of the molecules to be synthesized on the solid support. Various linkers can be used to attach the precursors of molecules to be synthesized to the solid phase support. Examples of linkers include aminobutyric acid, aminocaproic acid, 7-aminoheptanoic acid, 8-aminocaprylic acid, lysine, iminodiacetic acid, polyoxyethylene, glutamic acid, etc. In a further embodiment, linkers can additionally comprise one or more alanines or other amino acids as spacers.

In another embodiment, the “safety-catch amide linker” (SCAL) (see Patek, M. and Lebl, M. 1991, Tetrahedron Letters 1991, 32, 3891; International Patent Publication WO 92/18144, published Oct. 29, 1992) is introduced to the solid support.

In addition to the linkers described above, selectively cleavable linkers can be employed. One example is the ultraviolet light sensitive linker, ONb, described by Barany and Albericio (J. Am. Chem. Soc. 1985, 107, 4936). Other examples of photocleavable linkers are found in Wang (J. Org. Chem. 1976, 41, 32), Hammer et al. (Int. J. Pept. Protein Res. 1990, 36, 31), and Kreib-Cordonier et al. in “Peptides—Chemistry, Structure and Biology”, Rivier and Marshall, eds., 1990, pp. 895-897). Landen (Methods Enzym. 1977, 47, 145) used aqueous formic acid to cleave Asp-Pro bonds; this approach has been used to characterize T-cell determinants in conjunction with the Geysen pin synthesis method (Van der Zee et al. 1989, Eur. J. Immunol. 191: 43-47). Other potential linkers cleavable under basic conditions include those based on p-(hydroxymethyl)benzoic acid (Atherton et al. 1981, J. Chem. Soc. Perkin I: 538-546) and hydroxyacetic acid (Baleaux et al. 1986, Int. J. Pept. Protein Res. 28: 22-28). Geysen et al. (1990, J. Immunol. Methods 134: 23-33; International Publication WO 90/09395) reported peptide cleavage by a diketopiperazine mechanism. Preferred diketopiperazine linkages are disclosed in U.S. Pat. Ser. No. 5,504,265, which is hereby incorporated by reference in its entirety.

Enzyme-cleavable linkers can also be useful. An enzyme can specifically cleave a linker that comprises a sequence that is recognized by the enzyme. Thus, linkers containing suitable peptide sequences can be cleaved by a protease and linkers containing suitable nucleotide sequences can be cleaved by an endonuclease.

In certain instances, one can derivatize a portion (e.g., 10-90%) of the available resin functional groups with a cleavable linker using certain reaction conditions, and the remaining of the resin functional groups with a linker which is stable to the cleavage conditions to ensure that enough material will remain on the resin after cleavage for further study. This arrangement is particularly preferred when there are no coding tags. Combinations of linkers cleavable under different reaction conditions can also be used to allow selective cleavage of molecules from a single solid support bead.

In one embodiment, a cleavable linker can be used to release the synthetic test compound, or a portion thereof, for testing in a screening assay. In a preferred embodiment, the coding tags, if present, are each separately attached to the solid phase support via a cleavable linker that is stable to the conditions for release of the synthetic test compound. In another preferred embodiment, the scaffold linker is stable to the cleavage conditions for the coding linkers. In yet another embodiment, the coding tags are cleaved from the solid support prior to cleavage of the synthetic test compound.

One approach for the synthesis of encoded libraries involves linking the scaffolds and coding functional groups of the library together via a branched linker which also serves to link both precursors to the solid support. Depending on the structure of the linker, either the scaffold or the coding functional groups, or both, can be detached from the solid support for further study. One example of this approach of anchoring the scaffold and coding functional groups is to use Lys(SCAL) derivatized TentaGel.

A solid phase support linker for use in the present invention can further comprise a molecule of interest, which can be further derivatized to give a molecular library. The pre-attached molecule can be selected according to the methods described herein, or can comprise a structure known to embody desired properties. In a preferred embodiment, the scaffold linker is an amino acid.

An ionization linker has been used to enhance ionization of poorly- or non-ionizable molecules (Carrasco, M. R., et al. Tetrahedron Lett. 1997, 38, 6331-6334). The linker also provides a mass shift which overcomes signal overlap with matrix molecules. To effectively decode each bead with mass spectrometry, the linker should meet the following four criteria. First, the linker must be inert to the chemical reactions for library synthesis and stable under the conditions used for various biological screening. Second, the linker should be highly sensitive to the ionization method so that the final coding tags with different structures can be readily detected. Third, its cleavage must be clean and efficient. Fourth, the linker should have excellent solubility in the extraction solvent. A simple peptide-like linker that meets the above four criteria has been designed and synthesized on solid phase using the standard Fmoc chemistry (Fields, G. B., et al. Int. J. Peptide Protein Res. 1990, 35, 161-214). In principle, any chemically cleavable or photosensitive linkers can be used as the cleavable part as long as they are compatible with the library synthesis and screening. Methionine is preferred due to its clean and specific cleavage by cyanogen bromide (CNBr), and the final homoserine lactone product (Gross, E. et al. J. Biol. Chem. 1962, 237, 1856-1860) is chemically stable. This cleavage method has been successfully applied to single-bead analysis of peptides (Youngquist, R. S. et al. Rapid Commun. Mass Spectrom. 1994, 8, 77-81; Youngquist, R. S., et al. J. Am. Chem. Soc. 1995, 117, 3900-3906). Two phenylalanines are coupled to the methionine to increase the molecular weight of the linker. Finally, a linear hydrophilic molecule is introduced to the linker to enhance solubility of the coding tag in the extraction solvent (50% acetonitrile/water). The whole linker has excellent chemical stability, and is very suitable for MALDI-FTMS detection. The oxygen atoms, the amide bonds and the side chain of phenylalanines in the linker allow efficient formation of primarily sodiated species, and therefore provide efficient ionization.

In a preferred embodiment, the linker shown in Scheme 1 is used as the coding linker.

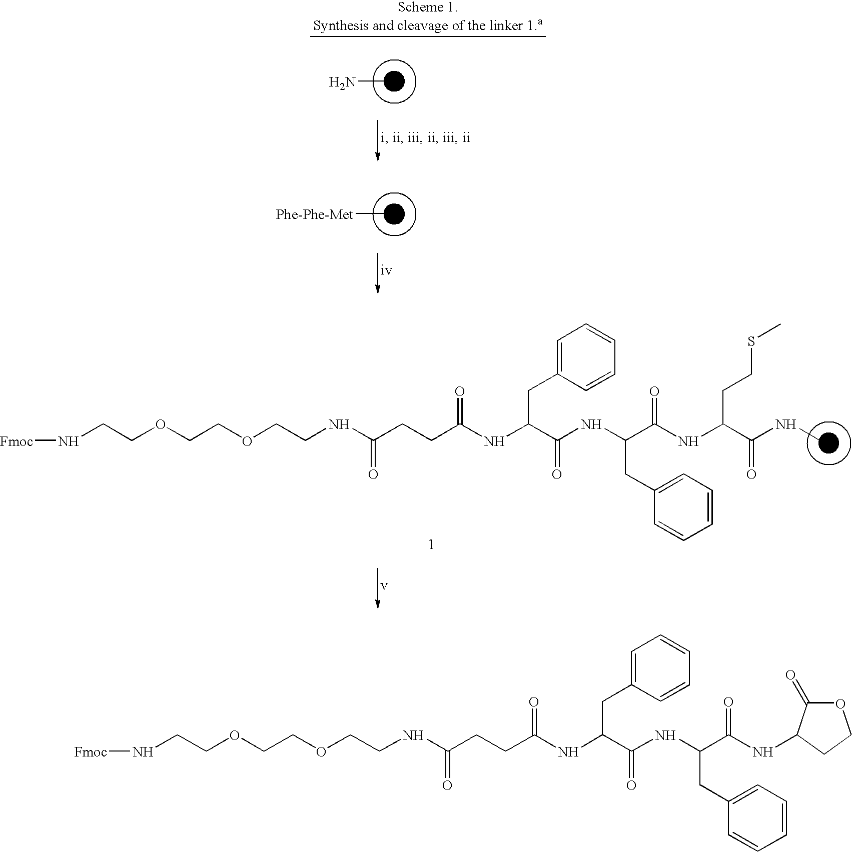

^{a} Reagents and conditions: (i) 3 equiv. of Fmoc-Met-OH, DIC and HOBt in DMF, rt, 1 h; (ii) 20% piperidine in DMF, rt, 30 min; (iii) 3 equiv. of Fmoc-Phe-OH, DIC and HOBt in DMF, rt, 1 h; (iv) 3 equiv. of Fmoc-NH(CH₂CH₂O)₂(CH₂)₂NHCO(CH₂)₂COOH, DIC and HOBt in DMF, rt, 3 h; (v) 0.25 M CNBr in 70% formic acid, rt, overnight.

E. Scaffolds

Scaffolds of the present invention can be a cyclic or bicyclic hydrocarbon, a steroid, a sugar, a heterocyclic structure, a polycyclic aromatic molecule, an amine, an amino acid, a multi-functional small molecule, a peptide or a polymer having various substituents at defined positions. Preferred scaffolds of the present invention include, but are not limited to, quinazoline, tricyclic quinazoline, purine, pyrimidine, phenylamine-pyrimidine, phthalazine, benzylidene malononitrile, amino acid, tertiary amine, peptide, polymer, aromatic compounds containing ortho-nitro fluoride(s), aromatic compounds containing para-nitro fluoride(s), aromatic compounds containing ortho-nitro chloromethyl, aromatic compounds containing ortho-nitro bromomethyl, lactam, sultam, lactone, pyrrole, pyrrolidine, pyrrolinone, oxazole, isoxazole, oxazoline, isoxazoline, oxazolinone, isoxazolinone, thiazole, thiozolidinone, hydantoin, pyrazole, pyrazoline, pyrazolone, imidazole, imidazolidine, imidazolone, triazole, thiadiazole, oxadiazole, benzofuran, isobenzofuran, dihydrobenzofuran, dihydroisobenzofuran, indole, indoline, benzoxazole, oxindole, indolizine, benzimidazole, benzimidazolone, pyridine, piperidine, piperidinone, pyrimidinone, piperazine, piperazinone, diketopiperazine, metathiazanone, morpholine, thiomorpholine, phenol, dihydropyran, quinoline, isoquinoline, quinolinone, isoquinolinone, quinolone, quinazolinone, quinoxalinone, benzopiperazinone, quinazolinedione, benzazepine and azepine. Scaffolds of the present invention also comprise at least two scaffold functional groups including, but not limited to, hydroxyl, carboxyl, amino, thiol, aldehyde, halogen, nitro, cyano, amido, urea, carbonate, carbamate, isocyanate, sulfone, sulfonate, sulfonamide, sulfoxide, etc., for attaching the scaffold building block. One of skill in the art can envision that other scaffolds, such as a single carbon atom, are also useful in the present invention.

In the following schemes 2-31, one of skill in the art will understand that radicals R₁, R₂, R₃, R₄ and R₅ can be, for example, hydrogen, alkyl, cycloalkyl, heterocyclyl, aryl and heteroaryl, all optionally substituted. One of skill in the art will further understand that radical Ar is an aryl, which can be, for example, phenyl, naphthyl, pyridyl and thienyl. In one embodiment, the library of compounds of the present invention is prepared using a quinazoline scaffold according to Scheme 2. The scaffold building blocks of such a quinazoline scaffold are encoded as shown in Scheme 3.

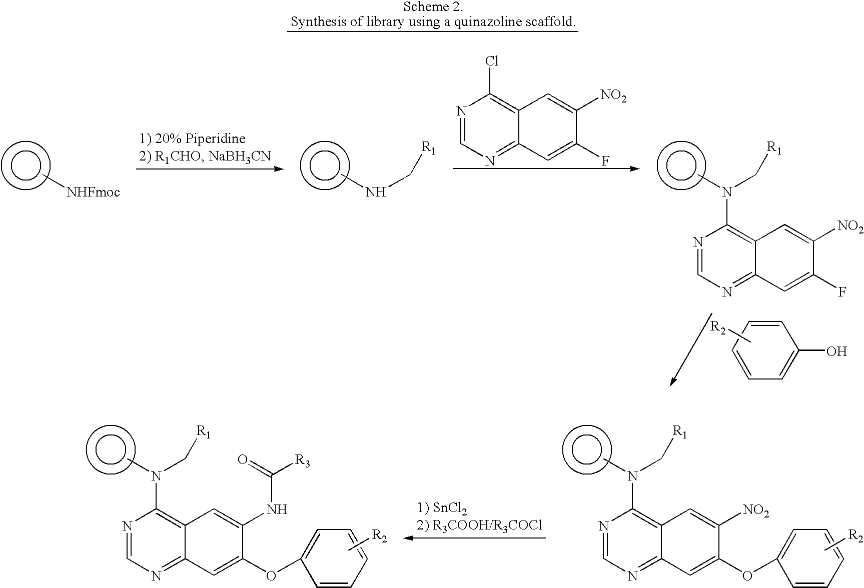

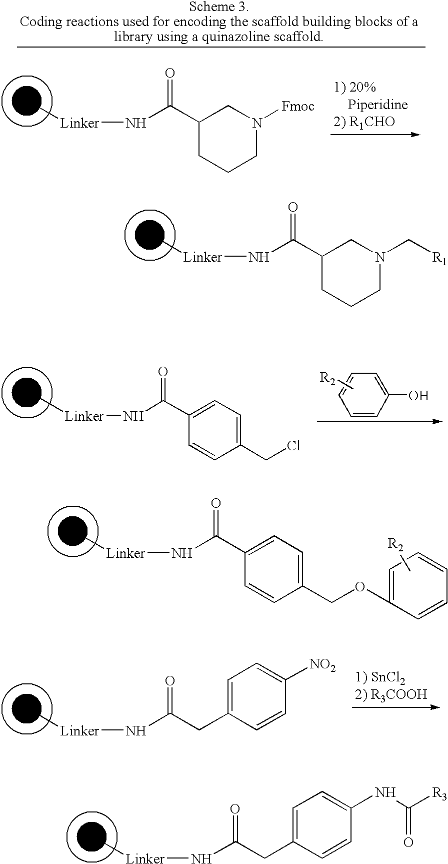

In one embodiment, the library of compounds of the present invention is prepared using a quinazoline scaffold according to Scheme 4. The scaffold building blocks of such a quinazoline scaffold are encoded as shown in Scheme 5.

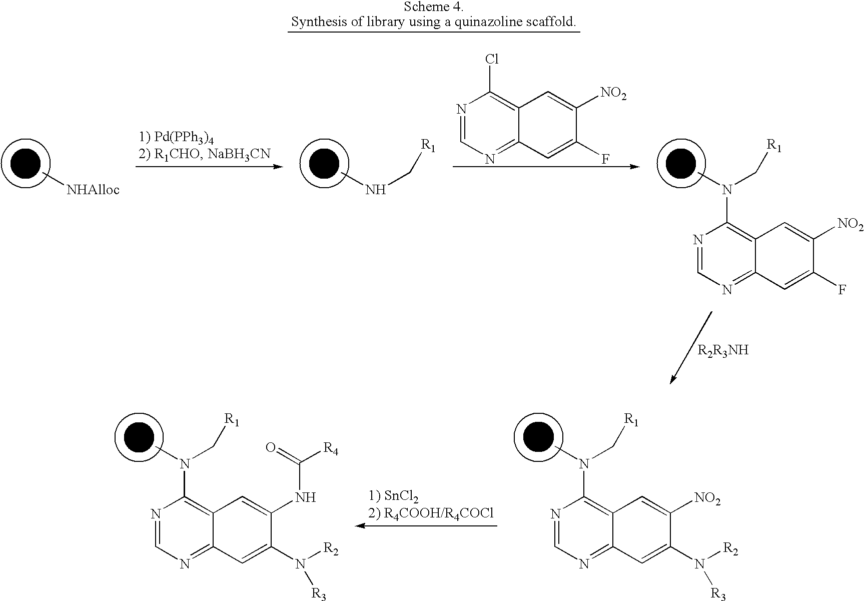

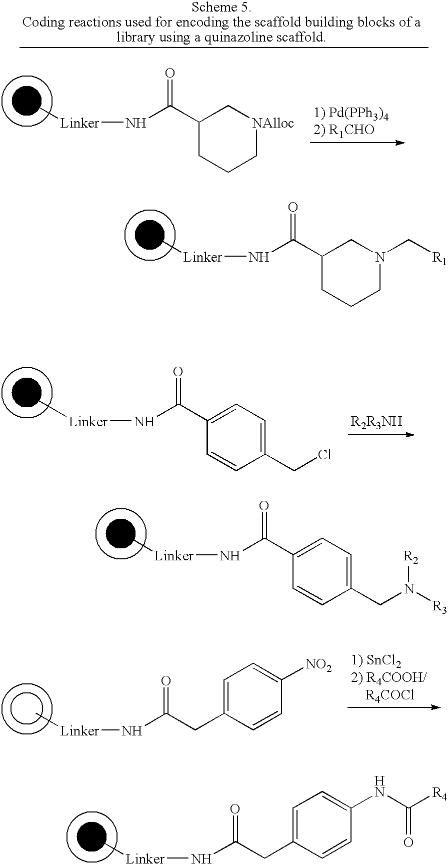

In one embodiment, the library of compounds of the present invention is prepared using a quinazoline scaffold according to Scheme 6. The scaffold building blocks of such a quinazoline scaffold are encoded as shown in Scheme 7.

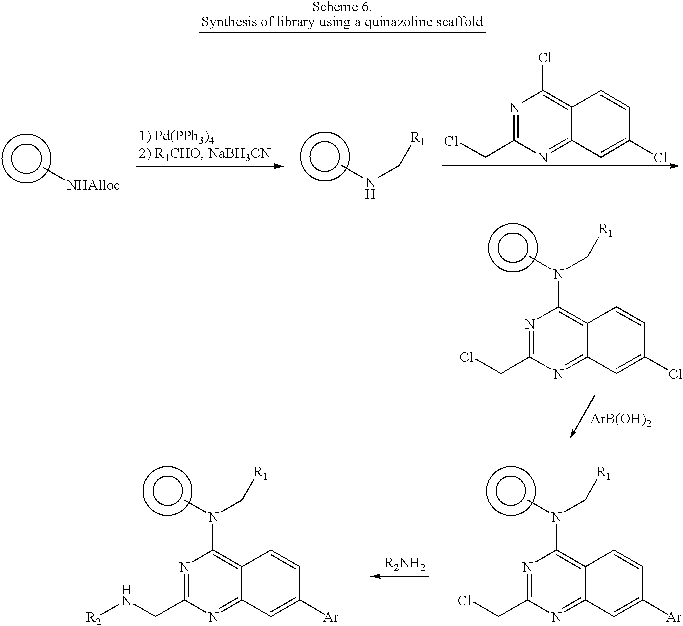

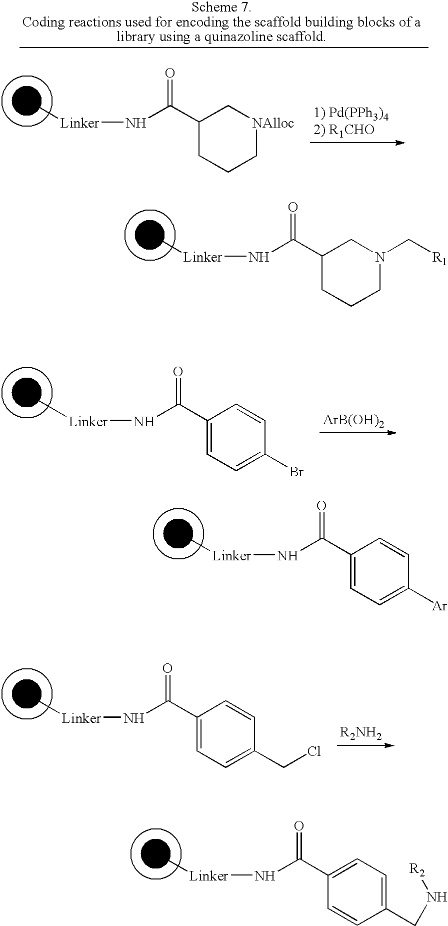

In one embodiment, the library of compounds of the present invention is prepared using a tricyclic quinazoline scaffold according to Scheme 8. The scaffold building blocks of such a tricyclic quinazoline scaffold are encoded as shown in Scheme 9.

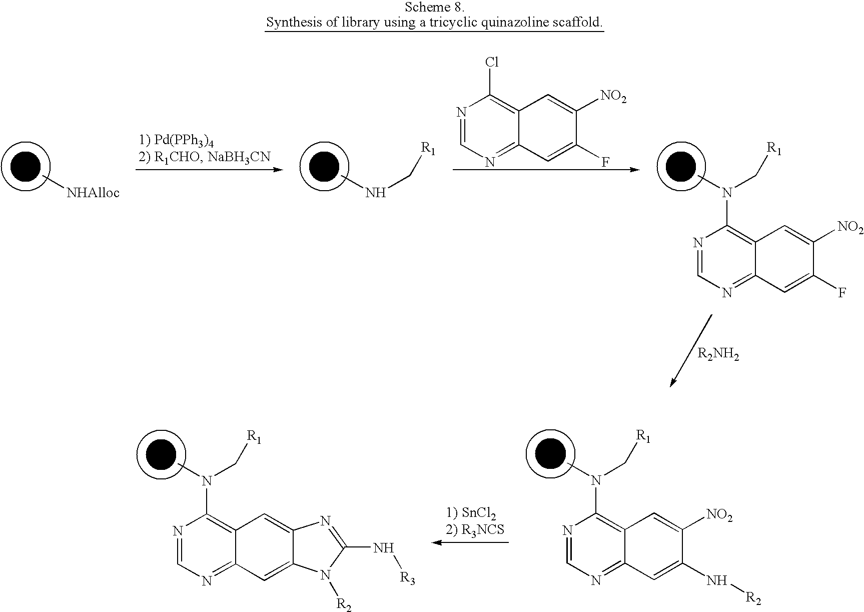

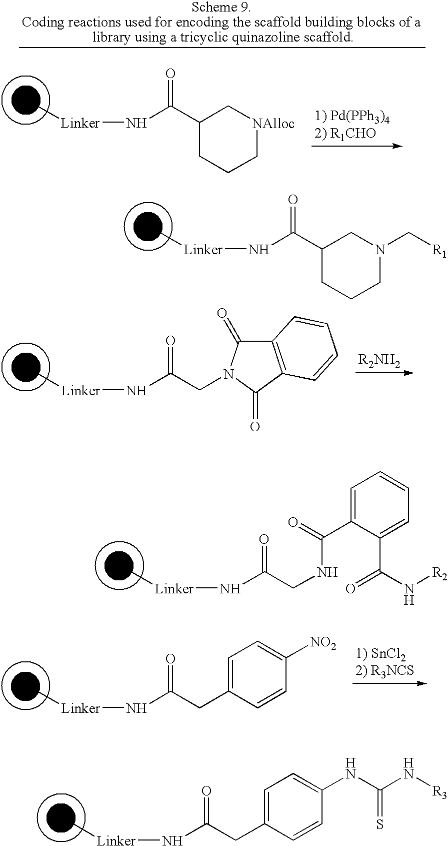

In one embodiment, the library of compounds of the present invention is prepared using a tricyclic quinazoline scaffold according to Scheme 10. The scaffold building blocks of such a tricyclic quinazoline scaffold are encoded as shown in Scheme 11.

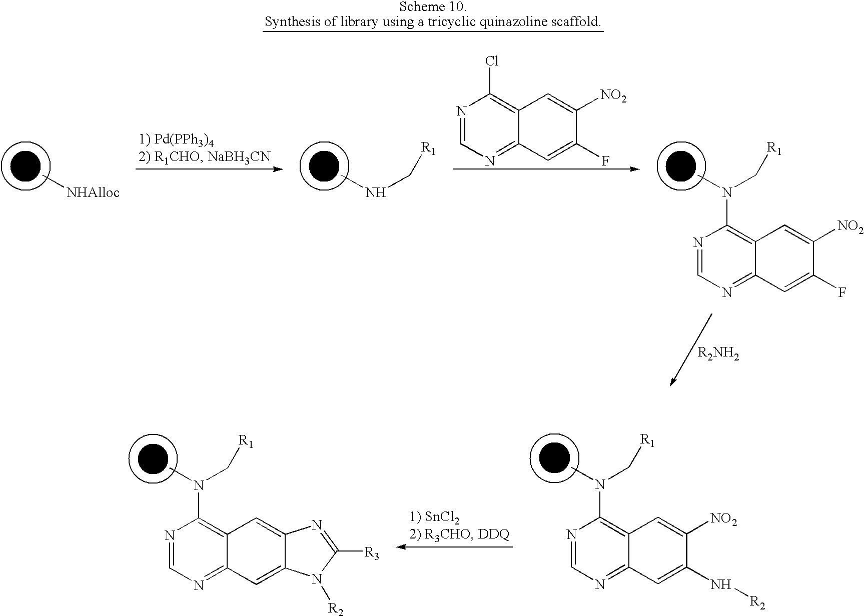

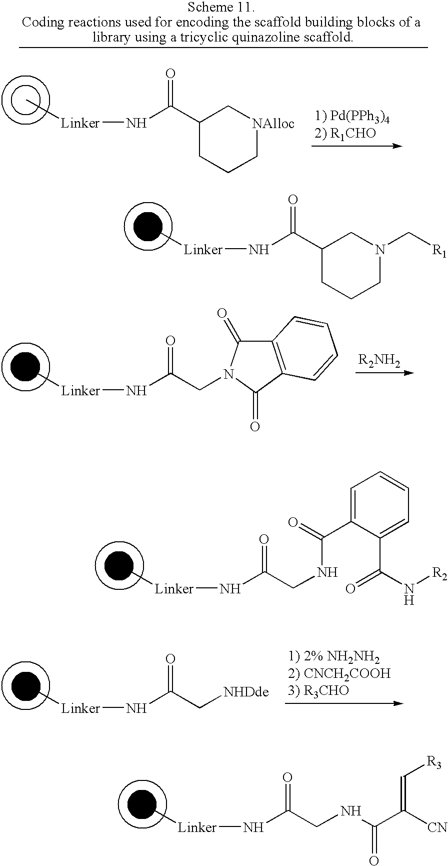

In one embodiment, the library of compounds of the present invention is prepared using the purine scaffold according to Scheme 12. The scaffold building blocks of the purine scaffold are encoded as shown in Scheme 13.

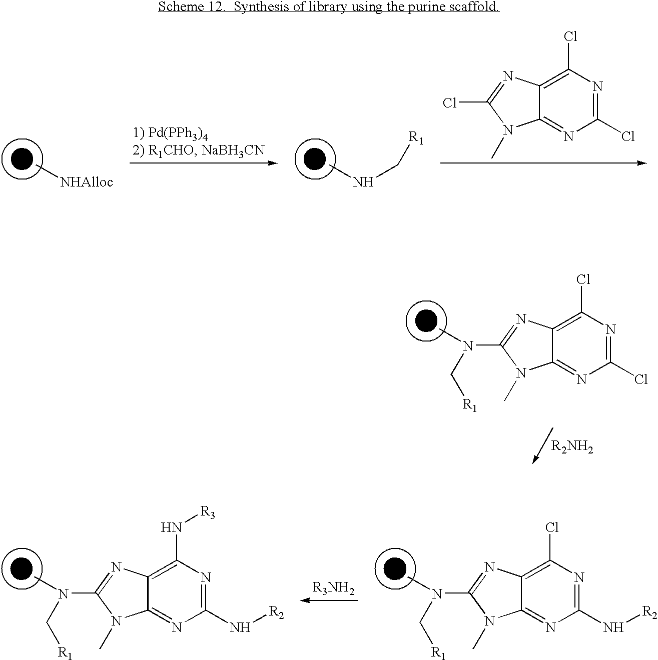

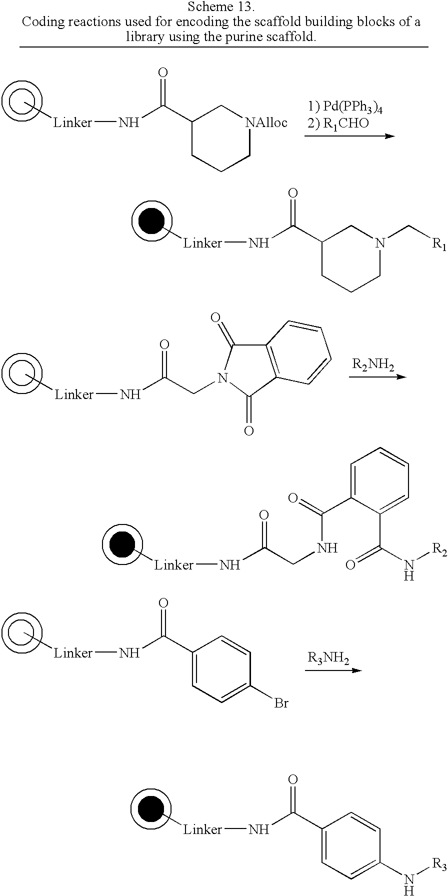

In one embodiment, the library of compounds of the present invention is prepared using the phenylamine-pyrimidine scaffold according to Scheme 14. The scaffold building blocks of the phenylamine-pyrimidine scaffold are encoded as shown in Scheme 15.

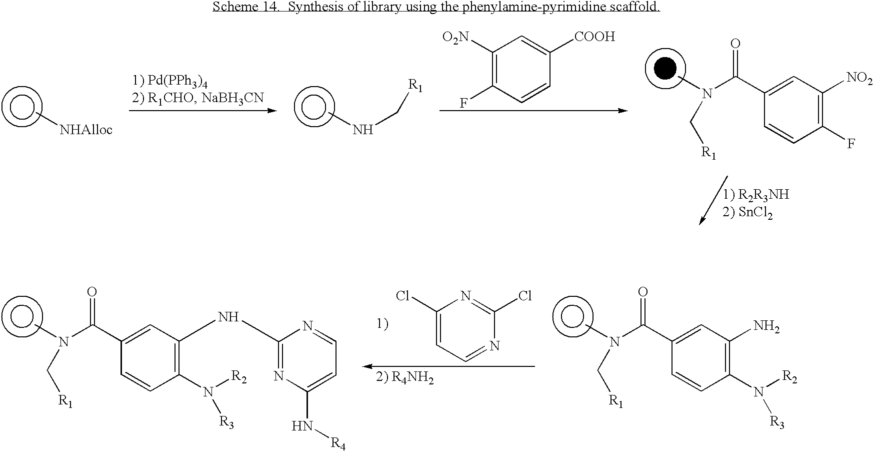

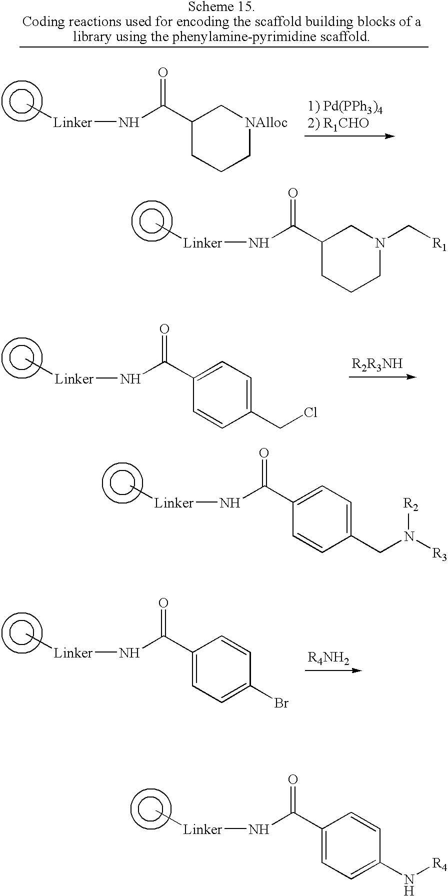

In one embodiment, the library of compounds of the present invention is prepared using the phthalazine scaffold according to Scheme 16. The scaffold building blocks of the phthalazine scaffold are encoded as shown in Scheme 17.

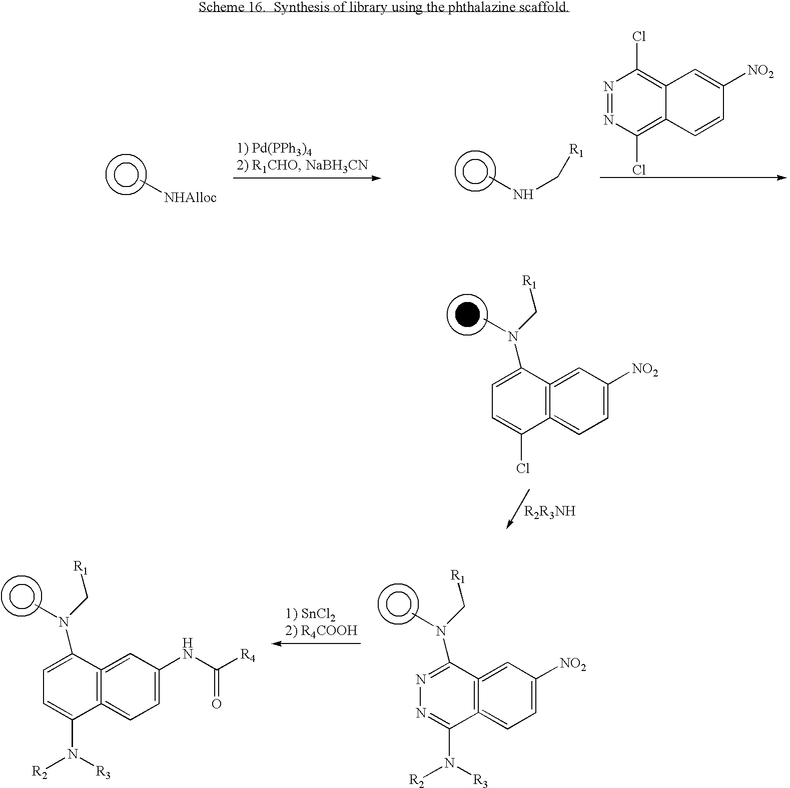

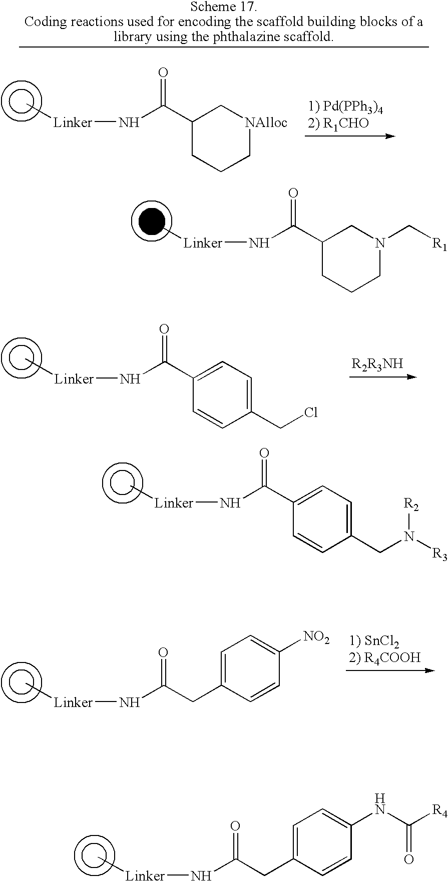

In one embodiment, the library of compounds of the present invention is prepared using the tertiary amine scaffold according to Scheme 18. The scaffold building blocks of the tertiary amine scaffold are encoded as shown in Scheme 19.

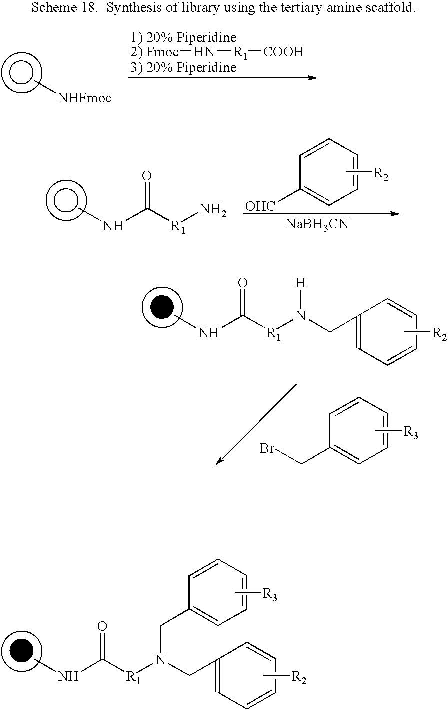

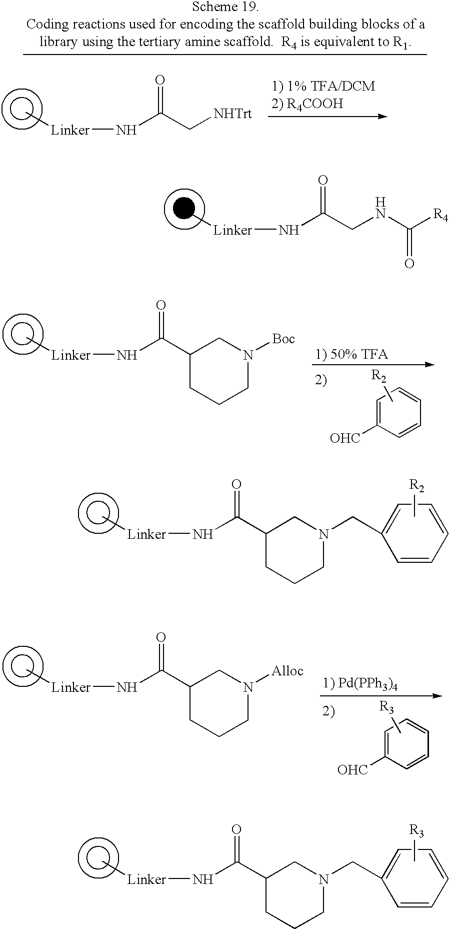

In one embodiment, the library of compounds of the present invention is prepared using the benzylidene malononitrile scaffold according to Scheme 20. The scaffold building blocks of the benzylidene malononitrile scaffold are encoded as shown in Scheme 21.

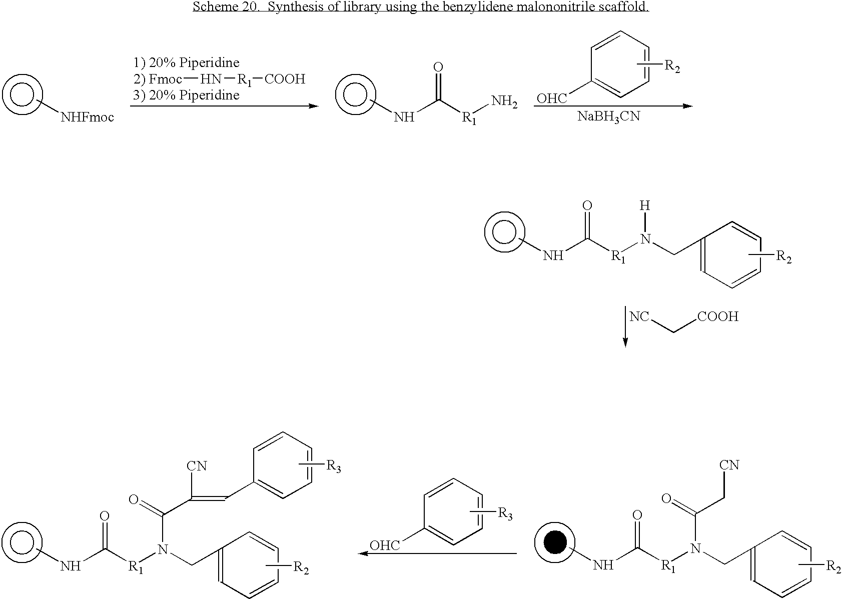

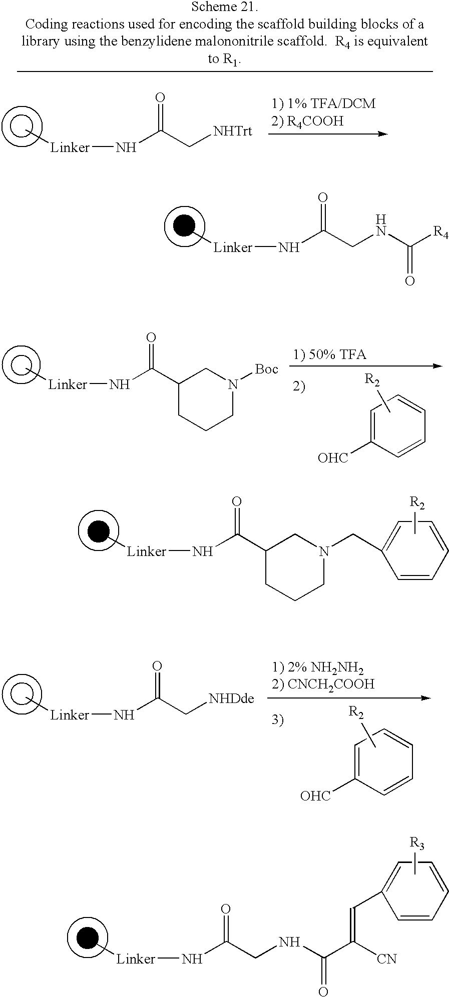

In one embodiment, the library of compounds of the present invention is prepared using the benzimidazole scaffold according to Scheme 22. The scaffold building blocks of the benzimidazole scaffold are encoded as shown in Scheme 23.

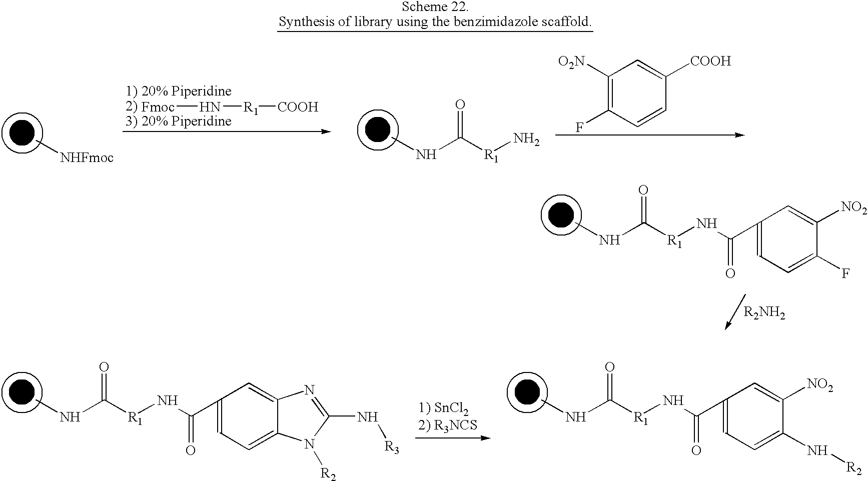

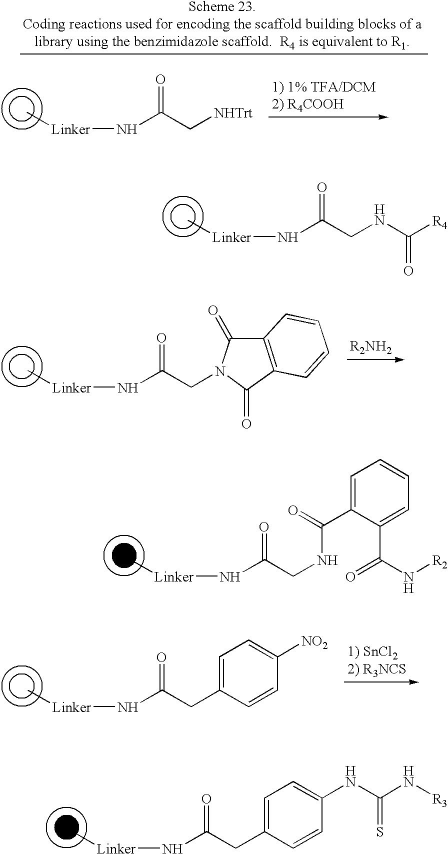

In one embodiment, the library of compounds of the present invention is prepared using the triazine scaffold according to Scheme 24. The scaffold building blocks of the triazine scaffold are encoded as shown in Scheme 25.

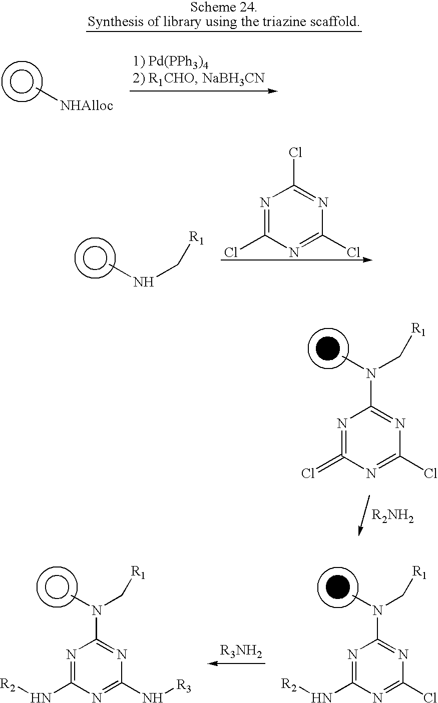

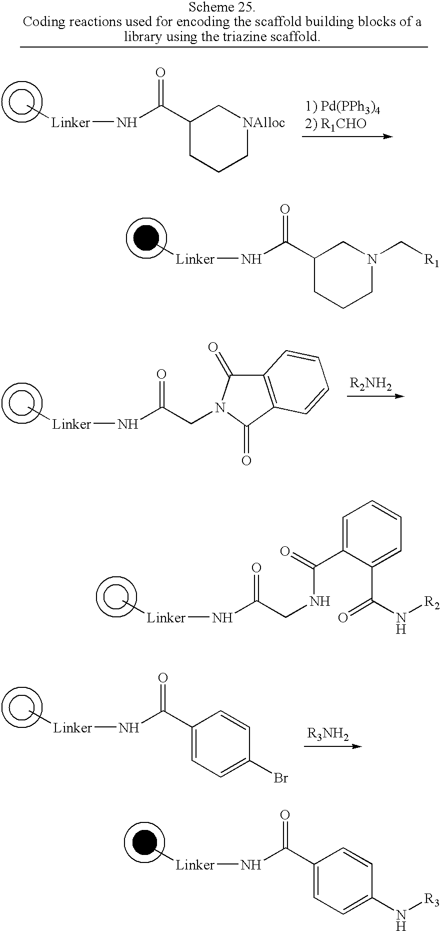

In one embodiment, the library of compounds of the present invention is prepared using an amino acid scaffold according to Scheme 26. The scaffold building blocks of such an amino acid scaffold are encoded as shown in Scheme 27.

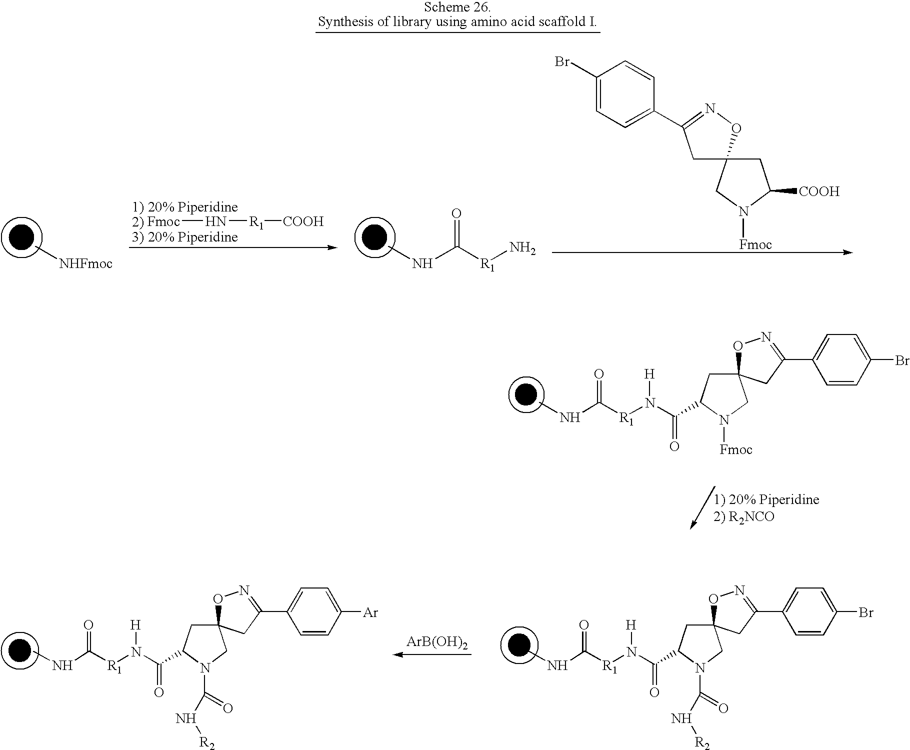

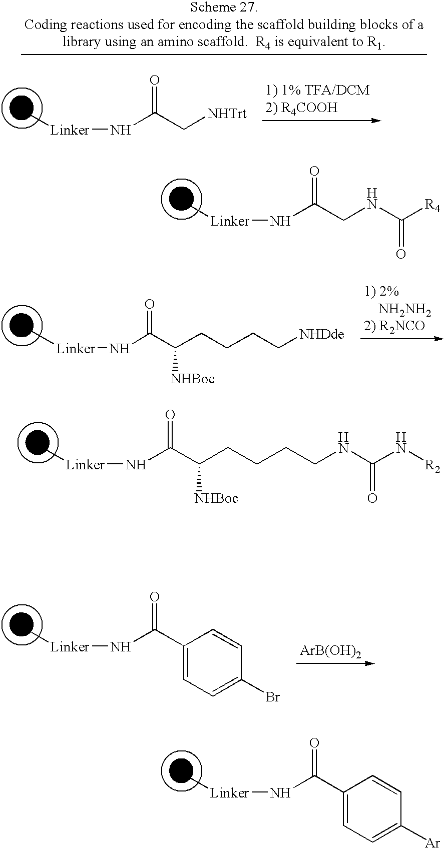

In one embodiment, the library of compounds of the present invention is prepared using the benzopyrazole scaffold according to Scheme 28. The scaffold building blocks of the benzopyrazole scaffold are encoded as shown in Scheme 29.

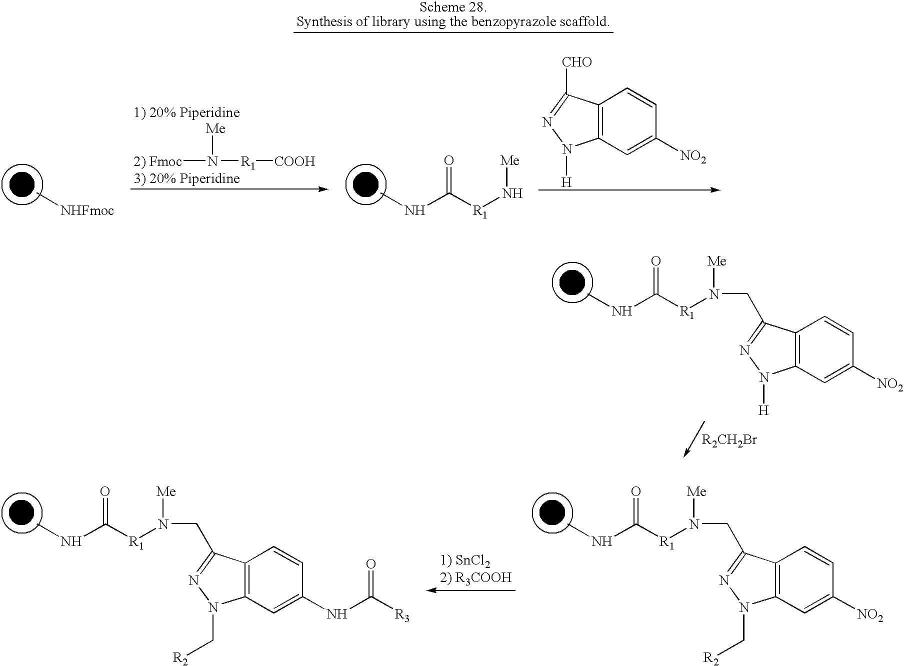

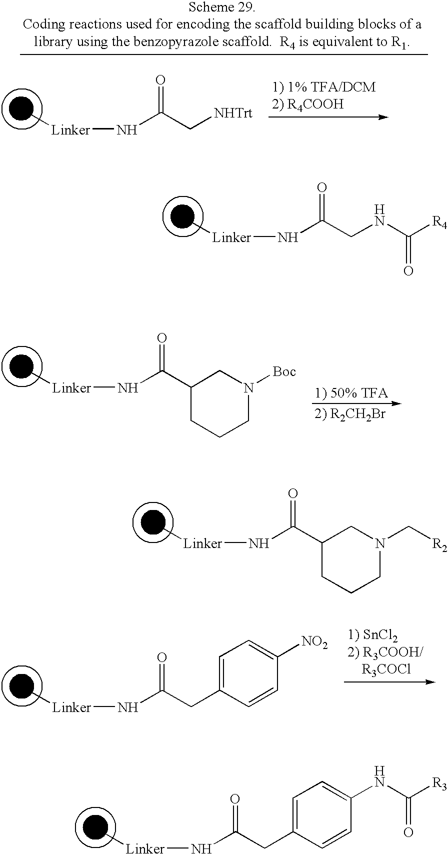

In one embodiment, the library of compounds of the present invention is prepared using an amino acid scaffold according to Scheme 30. The scaffold building blocks of such an amino acid scaffold are encoded as shown in Scheme 31.

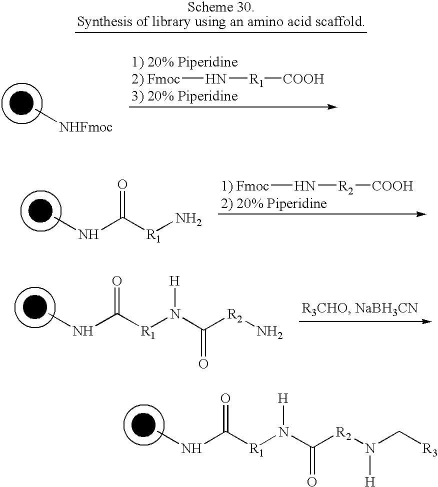

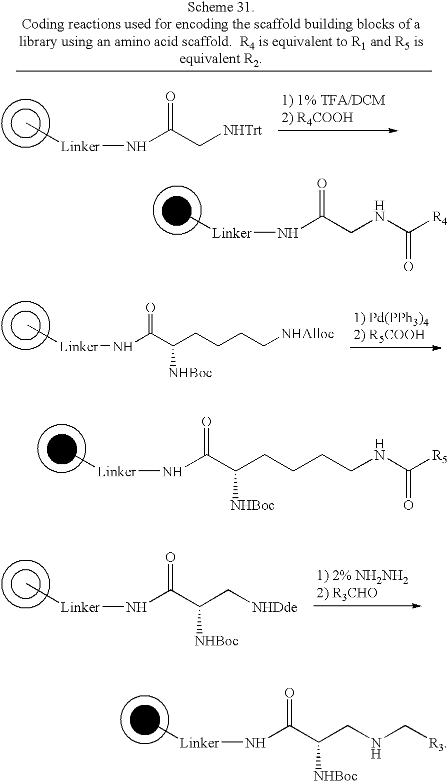

In a preferred embodiment, the scaffold is the same on each of the synthesis templates. In another preferred embodiment, at least two different scaffolds are used in the library. In yet another preferred embodiment, the scaffold is a member selected from the group consisting of quinazoline, tricyclic quinazoline, purine, pyrimidine, phenylamine-pyrimidine, phthalazine, benzylidene malononitrile, amino acid, tertiary amine, peptide, aromatic compounds containing ortho-nitro fluoride(s), aromatic compounds containing para-nitro fluoride(s), aromatic compounds containing ortho-nitro chloromethyl, aromatic compounds containing ortho-nitro bromomethyl, lactam, sultam, lactone, pyrrole, pyrrolidine, pyrrolinone, oxazole, isoxazole, oxazoline, isoxazoline, oxazolinone, isoxazolinone, thiazole, thiozolidinone, hydantoin, pyrazole, pyrazoline, pyrazolone, imidazole, imidazolidine, imidazolone, triazole, thiadiazole, oxadiazole, benzofuran, isobenzofuran, dihydrobenzofuran, dihydroisobenzofuran, indole, indoline, benzoxazole, oxindole, indolizine, benzimidazole, benzimidazolone, pyridine, piperidine, piperidinone, pyrimidinone, piperazine, piperazinone, diketopiperazine, metathiazanone, morpholine, thiomorpholine, phenol, dihydropyran, quinoline, isoquinoline, quinolinone, isoquinolinone, quinolone, quinazolinone, quinoxalinone, benzopiperazinone, quinazolinedione, benzazepine and azepine. Other scaffolds useful in the present invention will be apparent to one of skill in the art.

F. Split-Mix Methodology

In another preferred embodiment, the library of compounds is prepared via a split-mix methodology. In another aspect of the present invention, the method of the present invention for preparing a library of compounds via the split-mix methodology, comprises: a) providing a population of individual synthesis templates each comprising a solid support, wherein the solid support has an interior portion and an exterior portion each with a plurality of reactive functional groups, wherein the solid support is linked to a scaffold via a scaffold linker, wherein the scaffold has at least two scaffold functional groups, and wherein at least two coding tag precursors, each comprising a coding functional group and a coding linker, are attached to the solid support; b) splitting the population of synthesis templates into two or more separate pools; c) contacting the population of synthesis templates with one or more first reactive components in the two or more separate pools such that a first scaffold functional group reacts with one of the first reactive components to afford a first scaffold building block, and a first coding functional group reacts with one of the first reactive components to afford a first coding building block, wherein the contacting step yields subsequent synthesis templates; d) mixing the subsequent synthesis templates from the two or more separate pools into a single pool; e) splitting the subsequent synthesis templates into two or more separate pools; f) contacting the subsequent synthesis templates in the two or more separate pools with a successive reactive component such that a subsequent scaffold functional group reacts with the successive reactive component to afford a subsequent scaffold building block, and a subsequent coding functional group reacts with the successive reactive component to afford a subsequent coding building block, wherein the contacting step yields further synthesis templates; g) repeating steps d)-f), wherein the further synthesis templates of step f) become the subsequent synthesis templates of step d), until the library of compounds has been prepared.

The synthesis of libraries of synthetic test compound via a split-mix methodology comprises repeating the following steps: (i) dividing the selected support into a number of portions which is at least equal to the number of different subunits to be linked; (ii) chemically linking one and only one of the subunits of the synthetic test compound with one and only one of the portions of the solid support from step (i), preferably making certain that the chemical link-forming reaction is driven to completion to the fullest extent possible; (iii) thoroughly mixing the solid support portions containing the growing synthetic test compound; (iv) repeating steps (i) through (iii) a number of times equal to the number of subunits in each of the synthetic test compound of the desired library, thus growing the synthetic test compound; (v) removing any protecting groups that were used during the assembly of the synthetic test compound on the solid support.

Preferably, the coding building blocks are synthesized in parallel with the synthetic test compound. In this instance, before or after linking the subunit of the synthetic test compound to the support in step (ii), one coding building block, that correspond(s) to the added subunit of the synthetic test compound, is separately linked to the solid support, such that a unique structural code, corresponding to the structure of the growing synthetic test compound, is created on each support. It can be readily appreciated that if an encoded library is prepared, synthesis of the coding building block must precede the mixing step, (iii).

The repetition of steps (i)-(iii) (see step (iv)) will naturally result in growing the synthetic test compound and, if the process is modified to include synthesis of coding building blocks, a coding building block in parallel with each step of the test compound.

In one embodiment, enough support particles are used so that there is a high probability that every possible structure of the synthetic test compound is present in the library. Such a library is referred to as a “complete” library. To ensure a high probability of representation of every structure requires use of a number of supports in excess, e.g., by five-fold, twenty-fold, etc., according to statistics, such as Poisson statistics, of the number of possible species of compounds. In another embodiment, especially where the number of possible structures exceeds the number of supports, not every possible structure is represented in the library. Such “incomplete” libraries are also very useful.

IV. Screening Methods

In addition to providing libraries of a great variety of chemical structures as synthetic test compound, and methods of synthesis thereof, the present invention further comprises a method for identifying a compound of the present invention that binds to a target, wherein the compound is attached to a solid support, the method comprising: a) contacting the compound according to the method described above with the target; and b) determining the functional effect of the compound upon the target. In a preferred embodiment, the target of the present invention is a biological target. In other embodiments, the target can be synthetic in nature, such as a photogenic receptor or other material with an intensity physical property.

In a preferred embodiment, the present invention provides a method for determining the functional effect on a target of a compound attached to a solid support, wherein the target is a protein kinase. In a more preferred embodiment, the target is a protein tyrosine kinase.

In another embodiment, the present invention provides a method for identifying a compound of the present invention that binds to a target, wherein the compound is not attached to a solid support, the method comprising: a) contacting the compound according to the method described above with the target; and b) determining the functional effect of the compound upon the target. In a preferred embodiment, the target of the present invention is a biological target. In other embodiments, the target can be synthetic in nature, such as a photogenic receptor or other material with an intensity physical property.

In a preferred embodiment, the present invention provides a method for determining the functional effect on a target of a compound not attached to a solid support, wherein the target is a protein kinase. In a more preferred embodiment, the target is a protein tyrosine kinase.

The methods of screening the test compounds of a library of the present invention identify ligands within the library that demonstrate a biological activity of interest, such as binding, stimulation, inhibition, toxicity, taste, etc. Other libraries can be screened according to the methods described infra for enzyme activity, enzyme inhibitory activity, and chemical and physical properties of interest. Many screening assays are well known in the art; numerous screening assays are also described in U.S. Pat. No. 5,650,489.

The ligands discovered during an initial screening may not be the optimal ligands. In fact, it is often preferable to synthesize a second library based on the structures of the ligands selected during the first screening. In this way, one may be able to identify ligands of higher activity.

A. Binding Assays

The present invention allows identification of synthetic test compounds that bind to acceptor molecules. As used herein, the term “acceptor molecule” refers to any molecule which binds to a ligand. Acceptor molecules can be biological macromolecules such as antibodies, receptors, enzymes, nucleic acids, or smaller molecules such as certain carbohydrates, lipids, organic compounds serving as drugs, metals, etc.

The synthetic test compound in libraries of the present invention can potentially interact with many different acceptor molecules. By identifying the particular ligand species to which a specific acceptor molecule binds, it becomes possible to physically isolate the ligand species of interest.

Because only a small number of solid support beads will be removed during each screening/detection/isolation step, the majority of the beads will remain in the bead pool. Therefore, the library can be reused multiple times. If different color or identification schemes are used for different acceptor molecules (e.g., with fluorescent reporting groups such as fluorescein (green), Texas Red (Red), DAPI (blue) and BODIPI tagged on the acceptors), and with suitable excitation filters in the fluorescence microscope or the fluorescence detector, different acceptors (receptors) can be added to a library and evaluated simultaneously to facilitate rapid screening for specific targets. These strategies not only reduce cost, but also increase the number of acceptor molecules that can be screened.

In the method of the present invention, an acceptor molecule of interest is introduced to the library where it will recognize and bind to one or more ligand species within the library. Each ligand species to which the acceptor molecule binds will be found on a single solid phase support so that the support, and thus the ligand, can be readily identified and isolated.

The desired ligand can be isolated by any conventional means known to those of ordinary skill in the art and the present invention is not limited by the method of isolation. For example, and not by way of limitation, it is possible to physically isolate a solid-support-bead ligand combination that exhibits the strongest physico-chemical interaction with the specific acceptor molecule. In one embodiment, a solution of specific acceptor molecules is added to a library which contains 105 to 107 solid phase support beads. The acceptor molecule is incubated with the beads for a time sufficient to allow binding to occur. Thereafter, the complex of the acceptor molecule and the ligand bound to the support bead is isolated. More specific embodiments are set forth in the following methods, which describe the use of a monoclonal antibody, as a soluble acceptor molecule to bind a ligand which is a peptide. It will be clear that these methods are readily adaptable to detect binding of any acceptor molecule.

In addition to using soluble acceptor molecules, in another embodiment, it is possible to detect ligands that bind to cell surface receptors using intact cells. The use of intact cells is preferred for use with receptors that are multi-subunit or labile or with receptors that require the lipid domain of the cell membrane to be functional. The cells used in this technique can be either live or fixed cells. The cells can be incubated with the library and can bind to certain peptides in the library to form a “rosette” between the target cells and the relevant bead-peptide. The rosette can thereafter be isolated by differential centrifugation or removed physically under a dissecting microscope.

Alternatively, one can screen the library using a panning procedure with cell lines such as (i) a “parental” cell line where the receptor of interest is absent on its cell surface; and (ii) a receptor-positive cell line, e.g., a cell line which is derived by transfecting the parental line with the gene coding for the receptor of interest. It is then possible to screen the library by the following strategy: (i) first depleting the library of its non-specific beads that will bind to the cells lacking the receptor by introducing a monolayer of parental cell line by the standard “panning technique” to leave receptor-specific non-binding beads, or irrelevant non-binding beads; (ii) removing the non-binding beads which will include both receptor-specific or irrelevant beads and loading them on a monolayer of receptor positive cell line in which the receptor-specific bead will bind to the receptor positive cell line; (iii) removing the remaining irrelevant non-binding beads by gentle washing and decanting; and (iv) removing the receptor-specific bead(s) with a micromanipulator, such as a micropipette.

As an alternative to whole cell assays for membrane bound receptors or receptors that require the lipid domain of the cell membrane to be functional, the receptor molecules can be reconstituted into liposomes where reporting group or enzyme can be attached.

The foregoing examples refer to synthetic test compound, and any of the compounds described previously, can be used in the practice of the instant invention. Thus, an acceptor molecule can bind to one of a variety of polyamides, polyurethanes, polyesters, polyfunctionalized structure capable of acting as a scaffolding, etc.

In one embodiment, the acceptor molecule can be directly labeled. In another embodiment, a labeled secondary reagent can be used to detect binding of an acceptor molecule to a solid phase support particle containing a ligand of interest. Binding can be detected by in situ formation of a chromophore by an enzyme label. Suitable enzymes include, but are not limited to, alkaline phosphatase and horseradish peroxidase. In a further embodiment, a two color assay, using two chromogenic substrates with two enzyme labels on different acceptor molecules of interest, can be used. Cross-reactive and singly-reactive ligands can be identified with a two-color assay.

Other labels for use in the present invention include colored latex beads, magnetic beads, fluorescent labels (e.g., fluoresceine isothiocyanate (FITC), phycoerythrin (PE), Texas red (TR), rhodamine, free or chelated lanthanide series salts, especially Eu³⁺, to name a few fluorophores), chemiluminescent molecules, radio-isotopes, or magnetic resonance imaging labels. Two color assays can be performed with two or more colored latex beads, or fluorophores that emit at different wavelengths. Labeled beads can be isolated manually or by mechanical means. Mechanical means include fluorescence activated sorting, i.e., analogous to FACS, and micromanipulator removal means.

In specific examples, enzyme-chromogen labels and fluorescent (FITC) labels are used.

Reactive beads can be isolated on the basis of intensity of label, e.g., color intensity, fluorescence intensity, magnetic strength, or radioactivity, to mention a few criteria. The most intensely labeled beads can be selected and the ligand attached to the bead can be structurally characterized directly e.g., by Edman sequencing or by mass spectral analysis if applicable, or indirectly by sequencing the coding peptide corresponding to the ligand of interest. In another embodiment, a random selection of beads with a label intensity above an arbitrary cut-off can be selected and subjected to structural analysis. One can potentially use modern image analysis microscopy to quantitate the color intensity, and hence precisely define the relative affinity of the ligand to the acceptor molecule prior to the structure analysis of the bead ligand. Similarly, quantitative immunofluorescence microscopy can be applied if the acceptor is tagged with a fluorescent label. In yet another embodiment, beads demonstrating a certain label intensity are selected for compositional analysis, e.g., amino acid composition analysis in the case of peptide ligands. A refinement library comprising a restricted set of amino acid subunits identified as important from the amino acid analysis can then be prepared and screened.

In another embodiment, the ligand(s) with the greatest binding affinity can be identified by progressively diluting the acceptor molecule of interest until binding to only a few solid phase support beads of the library is detected. Alternatively, stringency of the binding with the acceptor molecule, can be increased. One of ordinary skill would understand that stringency of binding can be increased by (i) increasing solution ionic strength; (ii) increasing the concentration of denaturing compounds such as urea; (iii) increasing or decreasing assay solution pH; (iv) use of a monovalent acceptor molecule; (v) inclusion of a defined concentration of known competitor into the reaction mixture; (vi) lowering the acceptor concentration; and (vii) decreasing the concentration of library compounds on the surface of the beads. Other means of changing solution components to change binding interactions are well known in the art.

In another embodiment, ligands that demonstrate low affinity binding may be of interest. These can be selected by first removing all high affinity ligands and then detecting binding under low stringency or less dilute conditions.

In a preferred embodiment, a dual label assay can be used. The first label can be used to detect non-specific binding of an acceptor molecule of interest to beads in the presence of soluble ligand. Labeled beads are then removed from the library, and the soluble ligand is removed. Then specific binding acceptor molecule to the remaining beads is detected. Ligands on such beads can be expected to bind the acceptor molecule at the same binding site as the ligand of interest, and thus to mimic the ligand of interest. The dual label assay provides the advantage that the acceptor molecule of interest need not be purified since the first step of the assay allows removal of non-specific positive reacting beads. In a preferred embodiment, fluorescent-labeled acceptor molecules can be used as a probe to screen a synthetic test library, e.g., using FACS.

B. Bioactivity Assays

The instant invention further provides assays for biological activity of a ligand-candidate from a library treated so as to remove any toxic molecules remaining from synthesis, e.g., by neutralization and extensive washing with solvent, sterile water and culture medium. The biological activities that can be assayed include toxicity and killing, stimulation and growth promotion, signal transduction, biochemical and biophysical changes, and physiological change.

In a preferred embodiment, the synthetic test compounds of the library are selectively cleavable from the solid-phase support, also referred to herein as “bead”. Preferably, the synthetic test compounds are attached to the separate phase support via multiple cleavable linkers to allow for more than one release and screening assay. In one embodiment, beads are prepared such that only a fraction of synthetic test compound are selectively cleavable. A library is treated with a cleaving agent such that cleavage of a fraction of synthetic test compound occurs while the coding tags remain intact. Examples of cleaving agents include, but are not limited to, UV light, acid, base, enzyme, or catalyst. In one embodiment, the library is treated so that 10-99% of the synthetic test compound are released. In a more preferred embodiment, 25-50% of the synthetic test compound are released. Where all synthetic test compound molecules are cleavable, non-quantitative cleavage can be effected by limiting the cleaving agent. In one aspect, exposure time and intensity of UV light is limited. In another embodiment, the concentration of reagent is limited. After treatment to effect cleavage, the library can be further treated, e.g., by neutralization, to make it biologically compatible with the desired assay. In practice, one of ordinary skill would be able to readily determine appropriate cleavage conditions for partial cleavage when all synthetic test compound molecules of the library are attached to solid phase by cleavable linkers or bonds. One of ordinary skill would further understand that the relative concentration of released synthetic test compound can be affected by varying the cleavage conditions.

Since the beads of the library are immobilized, a concentration gradient of a particular ligand-candidate will form. High concentrations of synthetic test compound will be found in proximity of the bead from which it was released. Thus, evidence of biological activity of interest, in proximity to a bead, will allow identification and isolation of the bead, and structural characterization by sequencing the coding molecule corresponding to the synthetic test compound or other technique. Identification of the synthetic test compound is possible because the coding tags remain intact and attached to the interior of the bead during the screening procedures, and each positive bead can be readily decoded. In another embodiment, the beads can be partitioned in microtiter wells (e.g., 10 beads/well) and a fraction of ligand-candidate released and tested for biological activity, thus eliminating the potential problem of diffusion. Different portions of synthetic test compound can be attached to solid phase support or bead via different cleavable linkers for sequential assays. Within these examples, the term “bead” refers to a separate phase support particle.

Biological assays with uncleaved synthetic test compound are also envisioned. The biological activity of whole synthetic test compound-coated beads can then be screened. In one aspect, a library can be introduced into an animal. Beads of interest can be isolated from a specific tissue. Beads can be isolated that were specifically absorbed after oral, nasal, or cutaneous administration. In a preferred embodiment, such beads are magnetic, or have some other identifying feature, and thus are readily isolated from the tissue. In another embodiment, immobilized ligand itself can elicit biochemical changes with appropriate surface receptors.

It will further be understood by one of ordinary skill in the art that any cell that can be maintained in tissue culture, either for a short or long term, can be used in a biological assay. The term “cell” as used here is intended to include prokaryotic (e.g., bacterial) and eukaryotic cells, yeast, mold, and fungi. Primary cells or lines maintained in culture can be used. Furthermore, applicants envision that biological assays on viruses can be performed by infecting or transforming cells with virus. For example, and not by way of limitation, the ability of a ligand to inhibit lysogenic activity of lambda bacteriophage can be assayed by identifying transfected E. coli colonies that do not form clear plaques when infected.

Methods of the present invention for assaying activity of a synthetic test compound molecule of a library are not limited to the foregoing examples; any assay system can be modified to incorporate the presently disclosed invention are useful.

C. Enzyme Mimics/Enzyme Inhibitors

The present invention further comprises libraries that are capable of catalyzing reactions, i.e., enzyme libraries; libraries of molecules that serve as co-enzymes; and libraries of molecules that can inhibit enzyme reactions. Thus, the present invention also provides methods to be used to assay for enzyme or co-enzyme activity, or for inhibition of enzyme activity.

Enzyme activity can be observed by formation of a detectable reaction product. In a particular embodiment, an enzyme from an enzyme library catalyzes the reaction catalyzed by alkaline phosphatase, e.g., hydrolysis of 5-bromo-4-chloro-3-indoyl phosphate (BCIP) and forms a blue, insoluble reaction product on the solid phase support.

In another embodiment, a zone of observable product, e.g., color or fluorescence, can be formed in a semi-solid matrix. A library is layered in a semi-solid matrix, e.g., agarose gel, and a chromogenic or other indicator substrate is added. Where an enzyme-bead complex from an enzyme library shows the desirable enzyme activity, a zone of product will form. For example, and not by way of limitation, a molecule from a library which is a horseradish peroxidase mimic, can be identified by adding a solution of aminoantipyrene (0.25 mg/ml; Kodak), phenol (8 mg/ml) and H₂O₂ (0.005%) in 0.1M phosphate buffer, pH 7.0. Beads with enzyme activity will form a purple zone of color. In another embodiment, beads with protease activity can be identified by addition of the well known colorimetric protease substrates.

Co-enzyme activity can be observed by assaying for the enzyme activity mediated by a co-enzyme, where the natural or common co-enzyme is absent.

Enzyme inhibitory activity can be detected with a partially-released synthetic test compound. In one example, and not by way of limitation, a library is layered in a semi-solid matrix that contains an enzyme. The library is treated to partially release ligand-candidate molecules. Where the molecule inhibits the enzyme activity, a zone lacking product can be identified. In one embodiment, the enzyme substrate is chromogenic, and a colored product is formed. Thus, presence of an enzyme inhibitor would yield a zone of no color. In another embodiment, inhibition of proteolysis of hemoglobin or an indicator enzyme such as alkaline phosphatase can be detected by the presence of an opaque zone in the semi-solid matrix. This is because presence of proteolysis inhibitor will prevent degradation of the hemoglobin or indicator enzyme.

It is well known to one of ordinary skill in the art that a synthetic test compound molecule that demonstrates enzyme activity, co-enzyme activity, or that inhibits enzyme activity, can be a peptide, a peptide mimetic, or one of a variety of small-molecule compounds.

D. Topological Segregation

The present invention further encompasses a method of segregating the coding molecules in the interior of the solid support and the test compound on the exterior, accessible to a macromolecular acceptor molecule of interest. The method encompasses the steps of synthesizing a linker, which in the preferred embodiment is a peptide. The linker contains a sequence which can be cleaved by methods known to one of skill in the art.

V. Therapeutic and Diagnostic Agents using Compounds of the Present Invention

Once the structure of a selected ligand is determined, a large amount of the compound can be synthesized chemically or biologically for confirmation of the results of the structural and screening experiments and other studies. Once a molecular structure of interest has been identified through library screening and structural analysis of active ligands, the present invention provides molecules that comprise the molecular structure for use in treatment or diagnosis of disease. The molecule identified through screening alone can provide a diagnostic or therapeutic agent, or can be incorporated into a larger molecule. A molecule comprising a structure with biological or binding activity can be termed an “effector molecule.” The present invention further provides libraries for use in various applications. The “effector” function of the effector molecule can be any of the functions described herein or known in the art.

The method described herein not only provides a new tool to search for specific ligands of potential diagnostic or therapeutic value, but also provides important information on a series of ligands of potentially vastly different structure which nonetheless are able to interact with the same acceptor molecule. Integrating such information with molecular modeling and modern computational techniques is likely to provide new fundamental understanding of ligand-receptor interactions.

The therapeutic agents of the present invention comprise effector molecules that will bind to the biologically active site of cytokines, growth factors, or hormonal agents and thereby enhance or neutralize their action, and that will block or enhance transcription and/or translation. In another embodiment, an effector molecule can be an enzyme inhibitor, e.g. an inhibitor for HIV protease will be an anti-HIV agent, and a Factor Xa inhibitor will be an anti-coagulant.

The therapeutic agents of the present invention include, for example, effector molecules that bind to a receptor of pharmacologic interest such as growth factor receptors, neurotransmitter receptors, or hormone receptors. These effector molecules can be used as either agonists or antagonists of the action of the natural receptor ligand.

Another application of effector molecules that bind to receptors would be to use the binding to building block the attachment of viruses or microbes that gain access to a cell by attaching to a normal cellular receptor and being internalized. Examples of this phenomenon include the binding of the human immunodeficiency virus to the CD4 receptor, and of the herpes simplex virus to the fibroblast growth factor receptor. Effector molecules that occupy the receptor could be used as pharmacologic agents to building block viral infection of target cells. Parasite invasion of cells could be similarly inhibited, after suitable effector molecules were identified according to this invention.

In another embodiment, an effector molecule comprising a structure that binds to an acceptor molecule of interest can be used to target a drug or toxin. In a preferred embodiment, the acceptor molecule of interest is a receptor or antigen found on the surface of a tumor cell, animal parasite, or microbe, e.g., bacterium, virus, unicellular parasite, unicellular pathogen, fungus or mold. In another embodiment, the targeted entity is an intracellular receptor.

In addition, it is possible that a few of the millions of synthetic test compound molecules in the pool can provide structures that have biological activity. One can isolate molecules that possess antitumor, anti-animal parasite, or antimicrobial, e.g., anti-weed, anti-plant parasite, antifungal, antibacterial, anti-unicellular parasite, anti-unicellular pathogen, or antiviral activities. In addition, some of these ligands can act as agonists or antagonists of growth factors, e.g., erythropoietin, epidermal growth factor, fibroblast growth factor, tumor growth factors, to name but a few, as well as hormones, neurotransmitters, agonists for the receptors, immunomodulators, or other regulatory molecules.

The therapeutic agents of the present invention also include effector molecules comprising a structure that has a high affinity for drugs, e.g., digoxin, benzodiazepam, heroine, cocaine, or theophylline. Such molecules can be used as an antidote for overdoses of such drugs. Similarly, therapeutic agents include effector molecules that bind to small molecules or metal ions, including heavy metals. Molecules with high affinity for bilirubin will be useful in treatment of neonates with hyperbilirubinemea.

In general, methods to identify molecules for therapy of diseases or illnesses such as are listed in the Product Category Index of The Physicians Desk Reference (PDR, 1993, 47th Edition, Medical Economics Data: Oradell, N. J., pp. 201-202) are useful. For example, an effector molecule with anti-cancer, antiparasite, anticoagulant, anticoagulant antagonist, antidiabetic agent, anticonvulsant, antidepressant, antidiarrheal, antidote, antigonadotropin, antihistamine, antihypertensive, antiinflammatory, antinauseant, antimigraine, antiparkinsonism, antiplatelet, antipruritic, antipsychotic, antipyretic, antitoxin (e.g., antivenin), bronchial dilator, vasodilator, chelating agent, contraceptive, muscle relaxant, antiglaucomatous agent, or sedative activity can be identified.

The therapeutic agents of the present invention can also contain appropriate pharmaceutically acceptable carriers, diluents and adjuvants. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, magnesium carbonate, magnesium stearate, sodium stearate, glycerol monostearate, talc, sodium chloride, dried skim milk, glycerol, propylene, glycol, water, ethanol and the like. These compositions can take the form of solutions, suspensions, tablets, pills, capsules, powders, sustained-release formulations and the like. Suitable pharmaceutical carriers are described in “Remington's Pharmaceutical Sciences” by E. W. Martin. Such compositions will contain an effective therapeutic amount of the active compound together with a suitable amount of carrier so as to provide the form for proper administration to the patient. While intravenous injection is a very effective form of administration, other modes can be employed, such as by injection, or by oral, nasal or parenteral administration.

A molecule comprising a structure determined according to the present invention can also be used to form diagnostic agents. The diagnostic agent can also be a molecule comprising one or more structures identified as a result of library screening, e.g., more than one polyamide sequence or polyalkane sequence. In addition, the diagnostic agent can contain any of the carriers described above for therapeutic agents.

As used herein, “diagnostic agent” refers to an agent that can be used for the detection of conditions such as, but not limited to, cancer such as T or B cell lymphoma, and infectious diseases as set forth above. Detection is used in its broadest sense to encompass indication of existence of condition, location of body part involved in condition, or indication of severity of condition. For example, a peptide-horseradish immunoperoxidase complex or related immunohistochemical agent could be used to detect and quantitate specific receptor or antibody molecules in tissues, serum or body fluids. Diagnostic agents can be suitable for use in vitro or in vivo. Particularly, the present invention will provide useful diagnostic reagents for use in immunoassays, Southern or Northern hybridization, and in situ assays.

In addition, the diagnostic agent can contain one or more markers such as, but not limited to, radioisotope, fluorescent tags, paramagnetic substances, or other image enhancing agents. Those of ordinary skill in the art would be familiar with the range of markers and methods to incorporate them into the agent to form diagnostic agents.

The therapeutic agents and diagnostic agents of the instant invention can be used for the treatment and/or diagnosis of animals, and more preferably, mammals including humans, dogs, cats, horses, cows, pigs, guinea pigs, mice and rats. Therapeutic or diagnostic agents can also be used to treat and/or diagnose plant diseases.

The diseases and conditions amenable to therapy or diagnosis with molecules discovered according to the present invention are as varied and wide-ranging as the permutations of structures in a library.

In another embodiment, low affinity-binding beads can be selected, and a limited library prepared based on the structure of the ligands on the beads. In another embodiment, a custom low affinity or high affinity support comprising one or a few ligands identified from the millions of synthetic test compound provided by the present invention can be used in chromatographic separations.

### VI. EXAMPLES

In Schemes 32-38 of the following examples, one of skill in the art will understand that radicals R¹, R², R³ and R⁴ can be, for example, hydrogen, alkyl, cycloalkyl, heterocyclyl, aryl and heteroaryl, optionally substituted, while Ar can be, for example, aryl or heteroaryl, optionally substituted.

### Example 1

### Preparation of Topologically Segregated Beads

The bi-phasic approach to prepare the topologically segregated bi-functional resin beads. To spatially separate the testing compounds from the coding tags on the same bead, topologically segregated bi-functional beads are first prepared by selective protection of the outer layer of the resin bead with a protecting group, e.g. Fmoc using a bi-phasic method. Briefly, the resin (e.g. TentaGel bead, Rapp Polymere, Tubingen, Germany) is first pre-swollen in water followed by reaction with Fmoc-OSu dissolved in organic solvent. As the swelling properties of the bead in DCM and water are very different, the water-soaked beads swell in DCM causing Fmoc-OSu to penetrate into the interior of the bead, resulting in Fmoc-derivatization of the bead-interior as well. It has been observed that by adding diethyl ether to DCM, the water-soaked TentaGel bead does not swell further when mixed with this organic mixture. It has been discovered that by adjusting the ratio of DCM/diethyl ether used in the derivatization step to 55/45, the rapid diffusion of the organic phase into the interior of the bead is prevented. By using less than one equivalent of Fmoc-OSu (e.g. 0.6 eqs) in the presence of DIEA at room temperature for 30 min, the amino groups on the outer layer of the resin bead can be preferentially protected, while substantial amounts of free amino groups in the interior of the bead can still be used for anchoring the coding tag. To microscopically visualize the location of the free amino groups on the topologically segregated bi-functional bead, the free amino groups are derivatized (Krchnak et al. 1988) with bromophenol blue (3′,3″,5′,5″-tetrabromophenolsulfonephthalein) indicator. To quantify the amount of Fmoc coupled to the bead, UV spectrophotometric analysis (Bennett et al. 1998), microsequencing of two different peptides on exterior and interior of a single bead, and amino acid analysis have been used.

Preparation of topographically segregated bifunctional TentaGel resin beads with 60% Boc outside and 40% Fmoc-Linker 1 inside (outside-Boc-inside-Fmoc-linker-bifunctional resin). TentaGel S NH₂ resin (1.0 g, 0.26 mmol) were swollen in water for 48 h. Water was drained, and a solution of Alloc-OSu (31.1 mg, 0.156 mmol) in DCM/diethyl ether (50 mL, v/v=55:45) mixture was added to the resin, followed by addition of DEEA (55μL, 0.312 mmol). The mixture was shaken vigorously for 1 h. The resin was washed three times with DCM and six times with DMF. Fmoc-Linker 1 was then built in the inner region of the resin beads using abovementioned procedure. The resin was washed three times with DCM. In the presence of argon, a solution of PhSiH₃ (770 μL, 6.24 mmol) in 4 mL DCM was added to the resin followed by a solution of Pd(PPh₃)₄ (75.1 mg, 0.065 mmol) in 12 mL DCM (Grieco, P., et al. J Peptide Res. 2001, 57, 250-256). The mixture was shaken in an argon atmosphere for 30 min. This process was repeated once. The resin was washed with DCM, DMF, and DCM three times for each. A solution of di-tert-butyl dicarbonate (1.19 mL, 5.2 mmol) in 10 mL DCM was added to the resin, followed by addition of DIEA (226.4 μL, 1.3 mmol). The mixture was shaken until ninhydrin test was negative. The obtained outside-Boc-inside-Fmoc-linker-bifunctional resin was washed with DCM, DMF, DCM and MeOH three times for each, and then dried in vacuo. The percentage of inner region was determined to be 39% using quantitative UV absorption analysis of the dibenzofulvene-piperidine adduct released by treatment with piperidine (Bennett, W. D., et al. Eds. In Advanced Chem Tech Handbook of combinatorial and solid-phase organic chemistry A guide to principles, products and protocols; Advanced ChemTech Inc.: Louisville, Ky., 1998, p330).

### Example 2

### Preparation and Cleavage of the Scaffold Linker

Synthesis of N-(9-fluorenylmethoxycarbonyl)-4-{2-[2-(2-aminoethoxyl)ethoxy]ethylamino}-4-oxo-butanoic acid (N-Fmoc-2,2′-(ethylenedioxy)bis(ethylamine) monosuccinamide, Fmoc-Ebes-OH) (Zhao, Z. G., et al. Bioconjugate Chem. 1999, 10, 242-430). 2,2′-(ethylenedioxy)bis(ethylamine) (1.46 mL, 10 mmol) was dissolved in 50 mL acetonitrile. A solution of succinic anhydride (1.0 g, 10 mmol) in 25 mL of acetonitrile was added dropwise under vigorous magnetic stirring over 1 h. The stirring was stopped after 3 h. After the waxy product settled down, the organic solvent was decanted and discarded. The product was redissolved in 100 mL 50% acetonitrile/water and chilled in an ice bath for 30 min. A solution of N-(9-fluorenylmethoxycarbonyloxy)succinimide (Fmoc-OSu, 4.4 g, 13 mmol) in 25 mL acetonitrile was added dropwise under vigorous magnetic stirring over 1 h. The pH of reaction mixture was maintained from 8-9 by adding DIEA throughout the reaction. The reaction was allowed to proceed overnight at room temperature. The solvents were removed in vacuo. The residue was dissolved in 100 mL of 5% aqueous NaHCO₃ solution and washed with ethyl acetate. The aqueous phase was then acidified with 1 M HCl to pH 2 and extracted with ethyl acetate (50 mL×3). The combined organic phase was washed with water and dried over anhydrous MgSO₄. The solution was concentrated to a small volume and diluted with hexanes. A white solid is obtained with a yield of 72.6%. Its purity was determined to be 98% by HPLC analysis based on the absorption at 254 nm. MALDI-FTMS analysis result (M+Na⁺): Calcd. 493.195. Found 493.198.

Solid phase synthesis of the cleavable linker. The sequence Phe-Phe-Met was synthesized on TentaGel S NH₂ resin using standard Fmoc chemistry with 1,3-diisopropylcarbodiimide (DIC) and HOBt as the activating system (Fields, G. B., et al. Int. J. Peptide Protein Res. 1990, 35, 161-214). Ninhydrin test was carried out to monitor amino acid coupling and Fmoc deprotection (Kaiser, E., et al. Anal. Biochem., 1970, 34, 595-598). After Fmoc deprotection of the last Phe, a mixture of Fmoc-Ebes-OH (3 equiv.), HOBt (3 equiv.) and DIC (3 equiv.) in DMF was added to the resin. The reaction mixture was gently shaken overnight until ninhydrin test was negative. The obtained Fmoc-linker-resin was washed with DMF, DCM, MeOH three times for each, and then dried in vacuo.

Cleavage of the scaffold linker. A single resin bead was transferred to a 200 μL polypropylene microcentrifuge tube in ethanol under a microscope. The solvent was evaporated in vacuo. 10 μL 0.25 M CNBr in 70% formic acid was added. The mixture was gently shaken overnight in the dark. The cleavage was stopped by freezing and lyophilizing to dryness.

### Example 3

### Determination of Relative Reactivity of Coding Functional Group Precursors

Fmoc-linker-resin (20 mg, 0.0052 mmol) was swollen in DMF overnight, followed by Fmoc deprotection. A mixture of the coding functional group precursor (0.0156 mmol), benzoic acid (1.91 mg, 0.0156 mmol), HOBt (4.22 mg, 0.0312 mmol), DIC (4.9 μL, 0.0312 mmol) and 0.4 mL DMF was agitated for 30 min, and then added to the resin. The reaction mixture was agitated until ninhydrin test was negative. The resin was washed with DMF, DCM and MeOH thoroughly. Fifty beads were randomly picked and divided into 5 groups for cleavage and MALDI-FTMS analysis.

TABLE 2
The relative reactivity of some coding functional group precursors

| Coding functional group | Relative | |
|---|---|---|
| precursors | reactivity | Encoding reactions |
| Benzoic acid | 1.00 | |
| Acrylic acid | 1.79 | Michael addition |
| Bromoacetic acid | 17.76 | Nucleophilic substitution |
| 4-Bromomethylphenylacetic acid | 1.39 | Nucleophilic substitution |
| 4-Chloromethylbenzoic acid | 1.81 | Nucleophilic substitution |
| 4-Maleimidobutyric acid | 0.47 | Nucleophilic substitution |
| N-Phthaloylglycine | 5.71 | Nucleophilic substitution |
| N-Tritylglycine | 1.55 | Amino acid coupling |
| N-Fmoc-nipecotic acid | 0.93 | Reductive alkylation |
| 3-Nitrophenylacetic acid | 2.75 | Aromatic reduction and |
| | | acylation |
| 4-Nitrophenylacetic acid | 0.70 | Aromatic reduction and |
| | | acylation |

### Example 4

### Decoding the Library

Following cleavage of the coding tags, the residue containing the cleaved coding tags was redissolved in 10 μL 50% acetonitrile/water. The sample was applied on the MALDI probe with 2 μL aliquots. Sodium dopant (0.01 M NaCl in 50% ethanol/water, 0.1 μL) was added to the probe tip followed by matrix solution (0.4 M of 2,5-dihydroxybenzoic acid in ethanol, 1 μL). Hot air was used to quickly crystallize the sample on the probe. All samples were analyzed using a commercial MALDI-FTMS instrument (IonSpec Corp., Irvine, Calif.), equipped with an external MALDI source, a 4.7 Tesla superconducting magnet, and a Nd:YAG laser operating at 355 nm.

### Example 5

### Synthesis and Screening of Model Encoded Library with a Benzene Scaffold

To demonstrate the utility and efficiency of the present encoding method, a simple encoded small molecule library was synthesized and screened against streptavidin using (R,S)-N-Fmoc-β-amino-5-fluoro-2-nitrobenzenepropanoic acid as the scaffold (Scheme 32). 4-(chloromethyl)benzoic acid, N-Fmoc-3-piperidinecarboxylic acid and 4-nitrophenylacetic acid were chosen to encode the para-nitro fluoride, Fmoc-protected amino group and nitro group on the scaffold, respectively. At the beginning of the library synthesis, a secondary amine was used as the first building block to replace both of the para-nitro fluoride on the scaffold and the chloride of the coding functional group 4-(chloromethyl)benzoic acid. The Fmoc protecting group was removed in this step simultaneously. A carboxylic acid or a Boc-protected amino acid was then coupled to the amino group on the scaffold as well as the coding functional group 3-piperidinecarboxylic acid, followed by reduction of nitro groups with Tin (II) chloride. In the last synthetic step, a carboxylic acid (anhydride, acyl chloride or sulfonyl chloride) reacted with the aryl amino groups of both the scaffold and the coding functional group. The Boc and acid-labile side-chain protecting groups of amino acids were removed by treatment with TFA after library synthesis.

In the model library synthesis, forty two secondary amines, forty two carboxylic acids or Boc-protected amino acids, and forty eight carboxylic acids (anhydrides, acyl chlorides or sulfonyl chlorides) were selected as the first (BB1), second (BB2), and third (BB3) building blocks, respectively. The molecular weights of coding functional groups were calculated prior to library synthesis to avoid any ambiguity in final decoding (Table 3).

TABLE 3
Structures of the building blocks (BB1, BB2, and BB3 for first, second, and third
synthetic steps, respectively) for library synthesis and the calculated molecular masses (MW)
of the corresponding coding tags.

| Entry | BB1 (R¹,R²) | MW | BB2 (R³)^{b} |
|---|---|---|---|
| 1 | 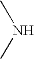 | 809.385 | CH₃COOH |
| 2 | 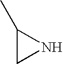 | 821.385 | 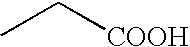 |
| 3 | 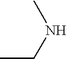 | 823.401 | 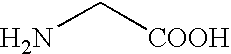 |
| 4 | 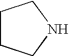 | 835.401 | 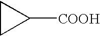 |
| 5 | 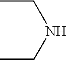 | 837.416 | 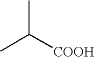 |
| 6 | 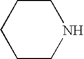 | 849.416 | 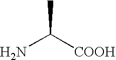 |
| 7 | 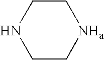 | 850.412 | 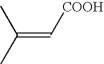 |
| 8 | 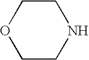 | 851.396 | 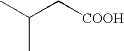 |
| 9 | 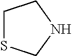 | 853.357 | 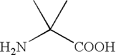 |
| 10 | 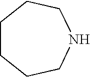 | 863.432 | 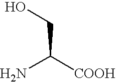 |
| 11 | 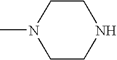 | 864.427 | 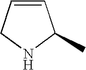 |
| 12 | 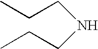 | 865.448 | 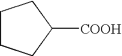 |
| 13 | 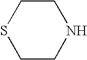 | 867.373 | 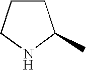 |
| 14 | 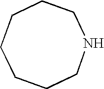 | 877.448 | 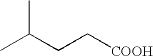 |
| 15 | 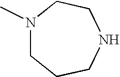 | 878.443 | 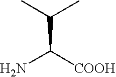 |
| 16 | 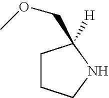 | 879.427 | 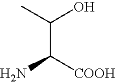 |
| 17 | 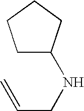 | 889.448 | 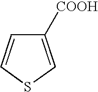 |
| 18 | 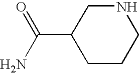 | 892.422 | 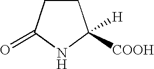 |
| 19 | 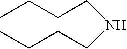 | 893.479 | 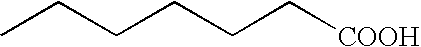 |
| 20 | 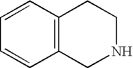 | 897.416 | 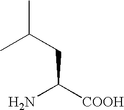 |
| 21 | 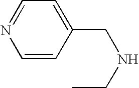 | 900.427 | 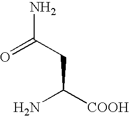 |
| 22 | 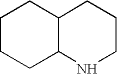 | 903.463 | 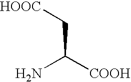 |
| 23 | 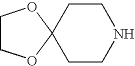 | 907.422 | 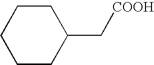 |
| 24 | 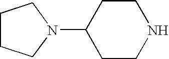 | 918.474 | 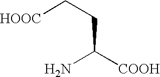 |
| 25 | 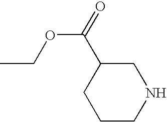 | 921.437 | 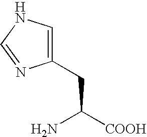 |
| 26 | 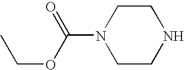 | 922.433 | 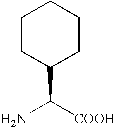 |
| 27 | 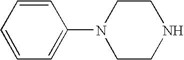 | 926.443 | 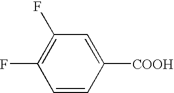 |
| 28 | 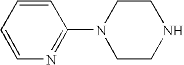 | 927.438 | 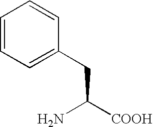 |
| 29 | 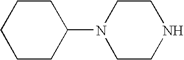 | 932.490 | 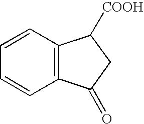 |
| 30 | 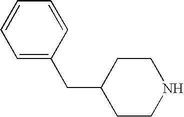 | 939.463 | 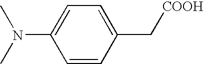 |
| 31 | 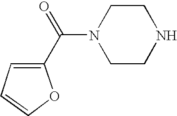 | 944.417 | 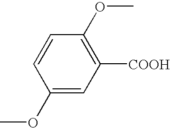 |
| 32 | 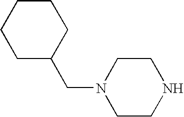 | 946.505 | 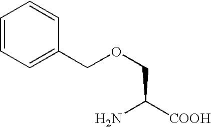 |
| 33 | 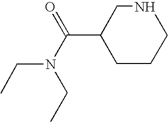 | 948.485 | 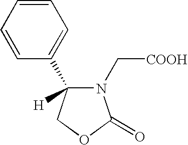 |
| 34 | 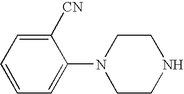 | 951.438 | 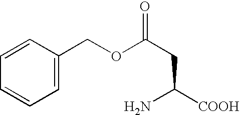 |
| 35 | 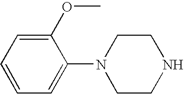 | 956.453 | 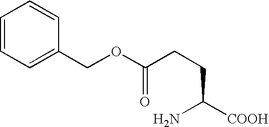 |
| 36 | 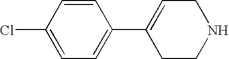 | 957.393 | 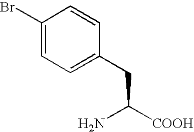 |
| 37 | 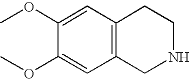 | 957.437 | 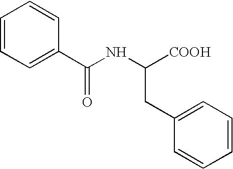 |
| 38 | 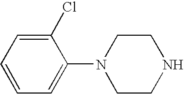 | 960.404 | 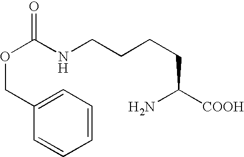 |
| 39 | 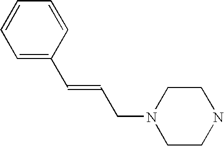 | 966.474 | 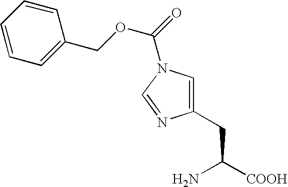 |
| 40 | 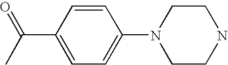 | 968.453 | 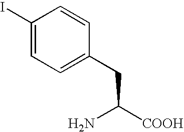 |
| 41 | 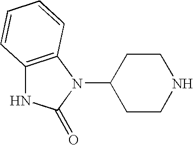 | 981.449 | 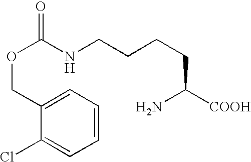 |
| 42 | 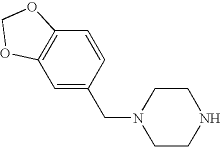 | 984.448 | 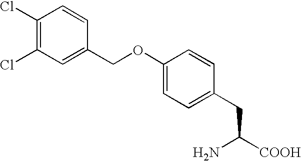 |

| Entry | MW | BB3 (R⁴) | MW |
|---|---|---|---|
| 1 | 801.380 | 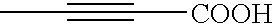 | 847.364 |
| 2 | 815.396 | (ClCH₂CO)₂O | 857.325 |
| 3 | 816.391 | 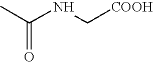 | 880.386 |
| 4 | 827.396 | 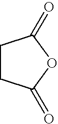 | 881.370 |
| 5 | 829.411 | 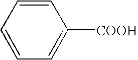 | 885.380 |
| 6 | 830.406 | 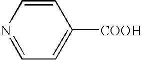 | 886.375 |
| 7 | 841.411 | 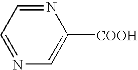 | 887.370 |
| 8 | 843.427 | 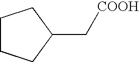 | 891.427 |
| 9 | 844.422 | 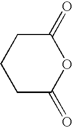 | 895.385 |
| 10 | 846.401 | 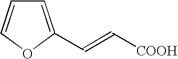 | 901.375 |
| 11 | 854.406 | 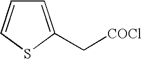 | 905.352 |
| 12 | 855.427 | 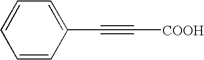 | 909.380 |
| 13 | 856.422 | 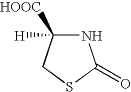 | 910.342 |
| 14 | 857.443 | 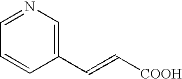 | 912.391 |
| 15 | 858.438 | 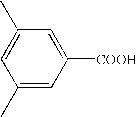 | 913.411 |
| 16 | 860.417 | 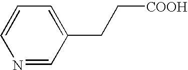 | 914.406 |
| 17 | 869.352 | 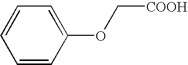 | 915.390 |
| 18 | 870.401 | 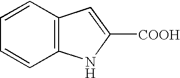 | 924.391 |
| 19 | 871.458 | 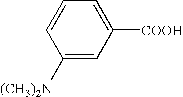 | 928.422 |
| 20 | 872.453 | 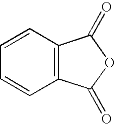 | 929.370 |
| 21 | 873.412 | 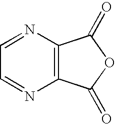 | 931.360 |
| 22 | 874.396 | 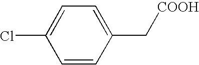 | 933.357 |
| 23 | 883.458 | 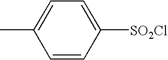 | 935.363 |
| 24 | 888.412 | 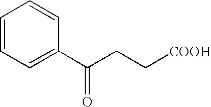 | 941.406 |
| 25 | 896.428 | 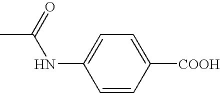 | 942.401 |
| 26 | 898.469 | 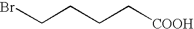 | 943.322 |
| 27 | 899.377 | 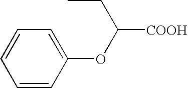 | 943.422 |
| 28 | 906.438 | 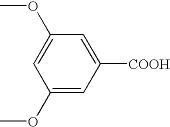 | 945.401 |
| 29 | 917.406 | 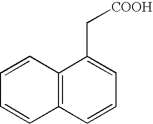 | 949.411 |
| 30 | 920.453 | 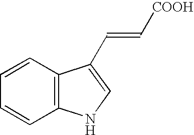 | 950.406 |
| 31 | 923.417 | 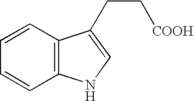 | 952.422 |
| 32 | 936.448 | 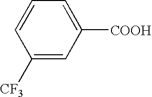 | 953.367 |
| 33 | 962.428 | 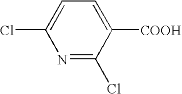 | 954.297 |
| 34 | 964.443 | 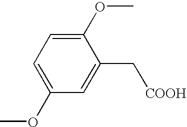 | 959.417 |
| 35 | 978.459 | 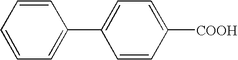 | 961.411 |
| 36 | 984.348 | 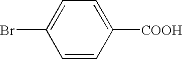 | 963.290 |
| 37 | 1010.464 | 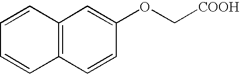 | 965.406 |
| 38 | 1021.501 | 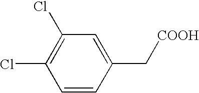 | 967.318 |
| 39 | 1030.465 | 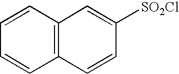 | 971.363 |
| 40 | 1032.334 | 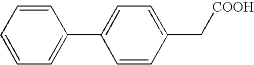 | 975.427 |
| 41 | 1055.462 | 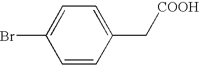 | 977.306 |
| 42 | 1080.402 | 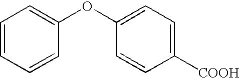 | 977.406 |
| 43 | | 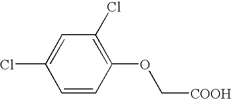 | 983.313 |
| 44 | | 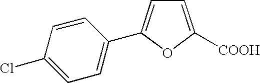 | 985.352 |
| 45 | | 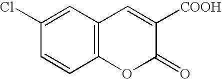 | 987.331 |
| 46 | | 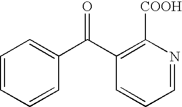 | 990.401 |
| 47 | | 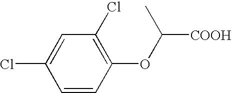 | 997.328 |
| 48 | | 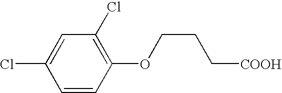 | 1011.344 |

^{a}One of the amino groups was protected with Boc during the synthesis.
^{b}All amino acids were N^{α}-Boc-protected, and their side-chains were protected with standard acid-labile protecting groups during the synthesis.

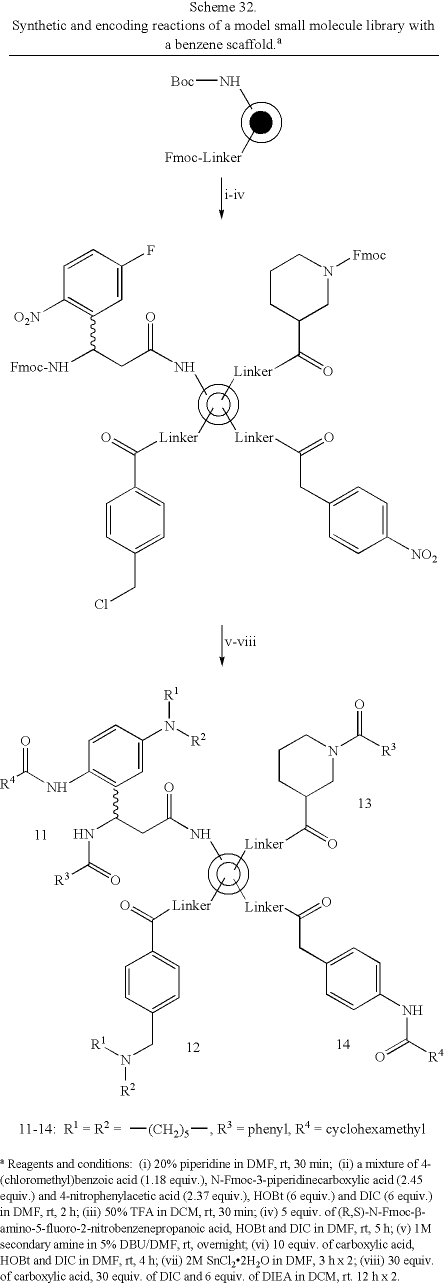

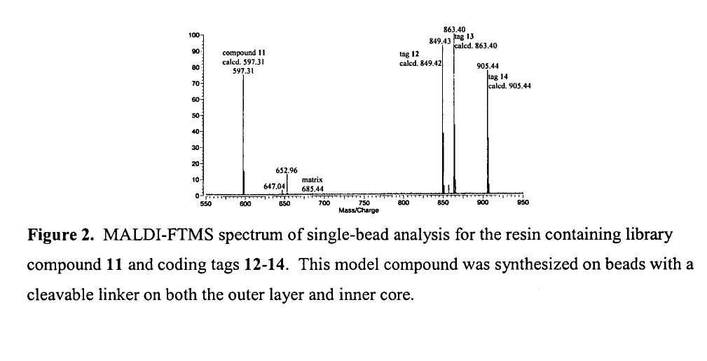

A randomly selected model compound (Scheme 32, compound 11) from this library was synthesized and encoded prior to the library synthesis. The model compound was linked to the solid support via methionine to make it releasable by CNBr. The decoding result is shown in FIG. 2. The obtained molecular masses of library compound 11 and three coding tags are consistent with the calculated values.

Synthesis of the model encoded library. Outside-Boc-inside-Fmoc-linker-bifunctional resin (0.5 g, 0.13 mmol) was swollen in DMF overnight, followed by Fmoc deprotection. A mixture of 4-(chloromethyl)benzoic acid (26.17 mg, 0.1534 mmol), N-Fmoc-3-piperidinecarboxylic acid (113.92 mg, 0.3185 mmol), 4-nitrophenylacetic acid (55.81 mg, 0.3081 mmol), HOBt (105.46 mg, 0.78 mmol), DIC (122.1 μL, 0.78 mmol) and 10 mL DMF was agitated for 30 min, and then added to the resin. The resulting mixture was agitated until ninhydrin test was negative. The resin was washed with DMF, MeOH and DCM three times for each, followed by Boc deprotection. A solution of (R,S)—N-Fmoc-β-amino-5-fluoro-2-nitrobenzenepropanoic acid (272.66 mg, 0.65 mmol), HOBt (87.88 mg, 0.65 mmol) and DIC (101.8 μL, 0.65 mmol) in 8 mL DMF was added to the resin. The reaction mixture was shaken until ninhydrin test was negative. The resin was filtered and washed with DMF, MeOH and DMF three times for each. The resin was then split into 42 aliquots, to each of which a solution of one of 42 secondary amines in 5% DBU/DMF was added. The reaction was allowed to proceed overnight. The resin beads were combined and washed with DMF, MeOH and DMF three times for each. The resin was split into 42 equal portions again. Each one of 42 carboxylic acids and Na-Boc-protected amino acids (0.031 mmol) was dissolved in a solution of HOBt (4.19 mg, 0.031 mmol) in 0.5 ml DMF followed by addition of DIC (4.9 μL, 0.031 mmol). The solutions were added to the 42 portions of resin individually. The reaction mixtures were shaken for 4 h. The resin beads were combined and washed with DMF, MeOH and DMF three times for each, followed by incubation with 10 mL of 2 M SnCl₂.2H₂O in DMF for 3 h. The reduction was repeated. After washing thoroughly with DMF, DCM, MeOH and DCM three times for each, the resin was split into 48 aliquots. To each aliquot of resin, a solution of one of 40 carboxylic acids (0.081 mmol) and DIC (12.7 μL, 0.081 mmol) in 0.7 ml DCM (or a solution of one of 8 anhydrides, acyl chlorides and sulfonyl chlorides (0.041 mmol) in 0.7 ml DCM) was added followed by addition of DIEA (2.8 μL, 0.016 mmol). The reaction was carried out for 12 h, and repeated. Complete acylation was confirmed by negative chloranil test (Vojkovsky, T. Pept. Res. 1995, 8, 236-237). The resin beads were combined, washed with DCM, DMF, DCM and MeOH three times for each, and then dried in vacuo. The bead-supported library was treated with a cleavage mixture consisting of TFA, TIS and water (v/v/v=95:2.5:2.5) for 2 h to remove the Boc and side-chain protecting groups of amino acids. After extensive washing with DCM, DMF, DCM and MeOH, the bead-supported library was stored in MeOH at 4° C.

After the library synthesis was completed, fifty resin beads were randomly picked for single bead analysis. Forty six of them were unambiguously decoded using MALDI-FTMS. No signals were obtained from 4 samples, probably due to the loss of the beads during sample transfer.

This 84 672-member library (42×42×48) was screened against streptavidin at an extremely dilute streptavidin-alkaline phosphatase conjugate concentration (1:100 000, or 50 pM) using an enzyme-linked colorimetric assay (Liu, R., et al. J. Am. Chem. Soc. 2002, 124, 7678-7680; Lam, K. S., et al. ImmunoMethods 1992, 1, 11-15).

Library screening. 0.2 mL of bead-supported library was transferred into a 10 mL disposable polypropylene column with a polyethylene frit. The beads were washed with water (4 mL×10), and then agitated with 4 mL PBST GN buffer (8.0 mM Na₂HPO₄, 1.5 mM KH₂PO₄, 137 mM NaCl, 2.7 mM KCl, pH 7.4, plus 0.1% Tween-20, 0.1% gelatin and 0.05% sodium azide) for 1 h. To the beads washed with PBST GN buffer (4 mL×5) streptavidin-alkaline phosphatase conjugate solution (2 mL) was added at a dilution of 1:100 000 (original concentration was 1 mg/mL) in PBST GN buffer. The mixture was incubated for 1 h. The beads were filtered and washed with TBS buffer (2.5 mM Tris-HCl, 13.7 mM NaCl, 0.27 mM KCl, pH 8.0, 4 mL), followed by washing with BCIP buffer (0.1 M Tris-HCl, 0.1 M NaCl, 2.34 mM MgCl₂, pH 8.8, 4 mL×2). A solution of the alkaline phosphatase substrate 5-bromo-4-chloro-3-indoyl phosphate (BCIP) in BCIP buffer (1.65 mg/mL, 2 mL) was added to the resin to develop color for 1 h. The enzymatic reaction was stopped by washing with PBST GN buffer (4 mL×5) and water (4 mL×5). The blue-colored beads were retrieved and treated with 6.0 M guanidine-HCl (pH 1.0) solution to strip the protein off the beads, and then washed twice with water. The indigo dye was removed by incubating the beads with acetone for 15 min. After washing with ethanol twice, the beads were ready for cleavage.

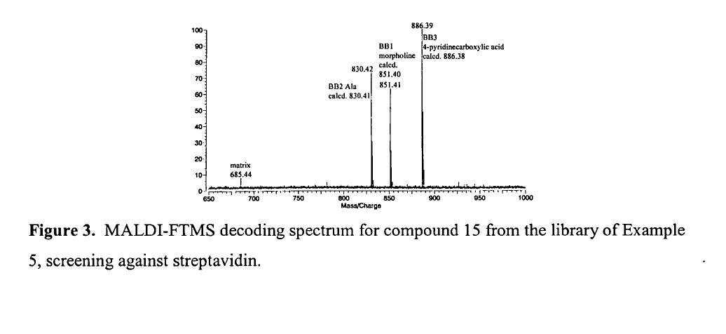

Twenty positive beads were isolated, and eighteen of them were successfully analyzed with MALDI-FTMS. A typical decoding spectrum is shown in FIG. 3. Seventeen streptavidin binding ligands with structural similarity have been identified. The decoding results are summarized in Table 4. At the position of first building block, morpholine was found seven times, and thiomorpholine was found three times. The second building block was quite variable, but a neutral amino acid appeared to be preferable. As the third building block, 4-pyridinecarboxylic acid was seen frequently (eight times), which has been found important for streptavidin binding (Liu, R., et al. J. Am. Chem. Soc. 2002, 124, 7678-7680). Its structural analogues, 3-pyridinepropanoic acid and pyrazinecarboxylic acid, were also observed in some of the ligands. The streptavidin binding affinity of these ligands was confirmed by re-synthesis of these compounds on regular TentaGel resin and re-screening against streptavidin using the same method. Two of these compound-beads (Scheme 33, compounds 15 and 16) stained very dark suggesting higher binding affinity than the others. The MALDI-FTMS spectrum for the coding tags of compound 15 is shown in FIGS. 3.

TABLE 4
Decoding results of positive beads obtained from streptavidin
binding assay.

| Entry BB1 (R¹ and R²) | | BB2 (R³) | BB3 (R⁴) |
|---|---|---|---|
| 1^{a} | Morpholine | Ala | 4-pyridinecarboxylic acid |
| 2 | Morpholine | Ala | 3-benzoyl-2- |
| | | | pyridinecarboxylic acid |
| 3 | Morpholine | Gly | pyrazinecarboxylic acid |
| 4 | Morpholine | Gly | 4-oxo-benzenebutanoic |
| | | | acid |
| 5 | Morpholine | Ser | 4-oxo-benzenebutanoic |
| | | | acid |
| 6 | Morpholine | Gly | 3,5-dimethoxybenzoic |
| | | | acid |
| 7 | Thiomorpholine | acetic acid | 4-pyridinecarboxylic acid |
| 8 | Thiomorpholine | Ala | 4-pyridinecarboxylic acid |
| 9 | Thiomorpholine | Ala | [1,1′-biphenyl]-4- |
| | | | carboxylic acid |
| 10 | Ethyl 1-piperazine | (S)-α-amino- | 4-pyridinecarboxylic acid |
| | carboxylate | cyclohexaneacetic | |
| | | acid | |
| 11 | Ethyl 1-piperazine | Leu | 4-pyridinecarboxylic acid |
| | carboxylate | | |
| 12 | Ethyl 1-piperazine | Ser | 3-pyridinepropanoic acid |
| | carboxylate | | |
| 13 | 1-(2-pyridyl)piperazine | Gly | 4-pyridinecarboxylic acid |
| 14 | 1-(2-cyanophenyl) | Leu | 4-pyridinecarboxylic acid |
| | piperazine | | |
| 15 | decahydroquinoline | Gly | 4-pyridinecarboxylic acid |
| 16 | 1-(2-furanylcarbonyl) | acetic acid | 4-oxo-benzenebutanoic |
| | piperazine | | acid |
| 17 | 1,4-dioxa-8- | Ser | 3-pyridinepropanoic acid |
| | azaspiro[4.5]decane | | |

^{a}The structure was found twice.

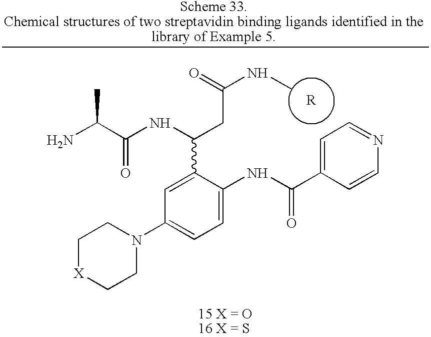

### Example 6

### Library Synthesis with a Quinoxaline Scaffold

A model compound (1, Scheme 34) from a randomly selected small molecule library was synthesized and encoded. The synthesis of the substituted 4-acyl-1,2,3,4-tetrahydroquinoxalin-2-ones based on a 4-fluoro-3-nitrobenzoic acid scaffold has been reported (Scheme 35) (Zaragoza and Stephensen, 1999). The synthetic and encoding reactions are shown in Scheme 34. The first scaffold functional group, i.e., the amino acid, can be readily coded by a pre-coupled carboxylic acid on the coding functional group Trt-Gly-OH (coding building block 2, Scheme 34). N-phthaloylglycine and 4-nitrophenylacetic acid are selected as coding functional groups to code the reactive components that react with the ortho-nitro fluoride and the nitro scaffold functional group.

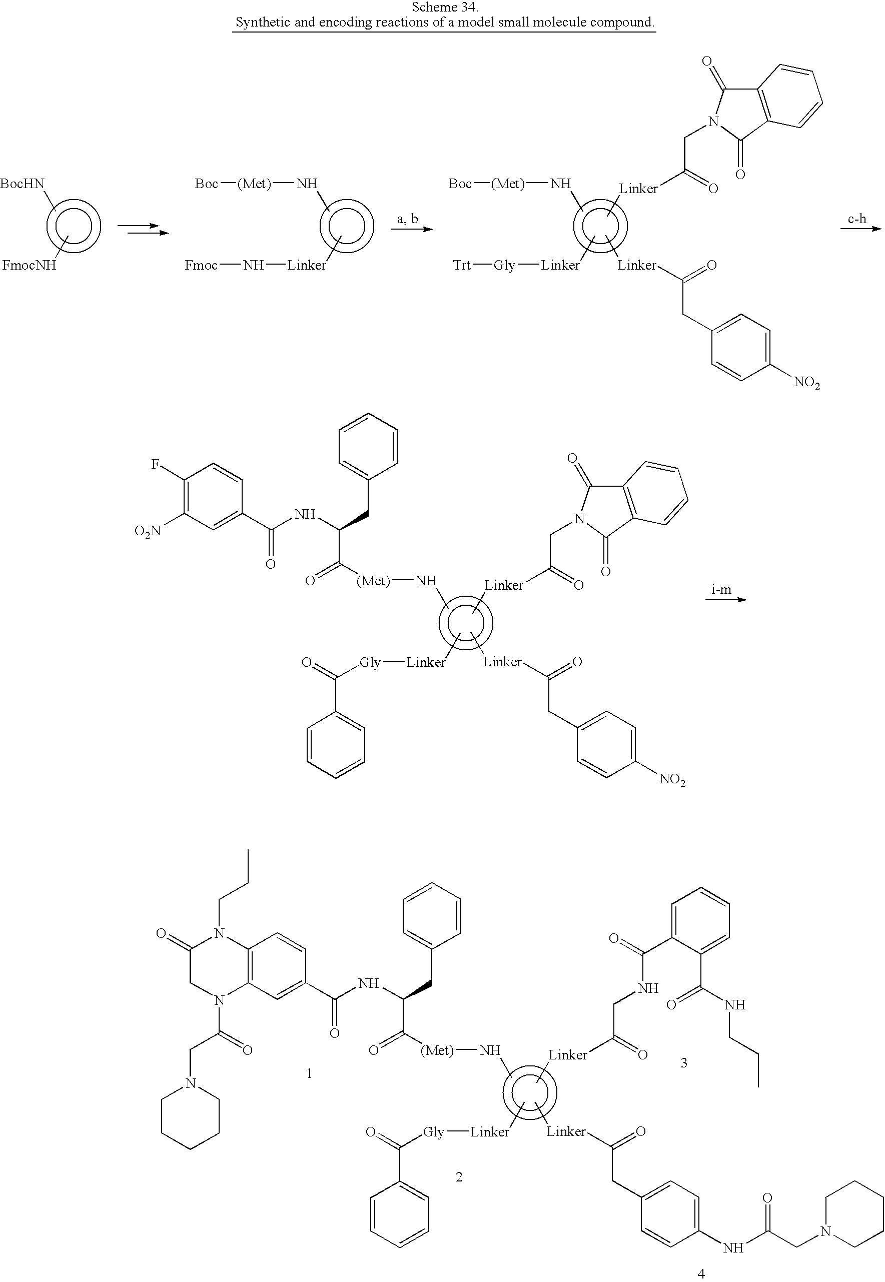

a) 20% piperidine/DMF, 20 min; b) a mixture of Trt-Gly-OH (2.01 equiv), N-phthaloylglycine (0.34 equiv) and 4-nitrophenylacetic acid (3.45 equiv), HOBt (6 equiv), DIC (6 equiv), DMF, 2 h; c) 1% TFA and 5% TIS in DCM, 2 min×4; d) benzoic acid (5 equiv), HOBt (5 equiv), DIC (5 equiv), DMF, 2 h; e) 50% TFA/DCM, 30 min; f) Boc-Phe-OH (5 equiv), HOBt (5 equiv), DIC (5 equiv), DMF, 2 h; g) 50% TFA/DCM, 30 min; h) 4-fluoro-3-nitrobenzoic acid (5 equiv), HOBt (5 equiv), DIC (5 equiv), DMF, 2 h; i) 0.25 M propylamine in 5% DIEA/DMF, overnight; j) 2M SnCl₂.H₂O in DMF, 3 h×2; k) chloroacetic anhydride (20 equiv), DIEA (4 equiv), DMF, overnight; 1) 10% DIEA/DMF, 6 h; m) 10% piperidine and 10% DIEA/DMF.

Scheme 35. Synthesis of substituted 4-acyl-1,2,3,4-tetrahydroquinoxalin-2-ones. One of skill in the art will understand that radical R′ can be, for example, hydrogen, alkyl, cycloalkyl, heterocyclyl, aryl and heteroaryl, all optionally substituted, that base can be, for example, an amine base, a nucleophilic base or a non-nucleophilic base, and the radical Nu-can be an amine, an alkoxide, an organometallic, or a carbon-based nucleophile, for example.

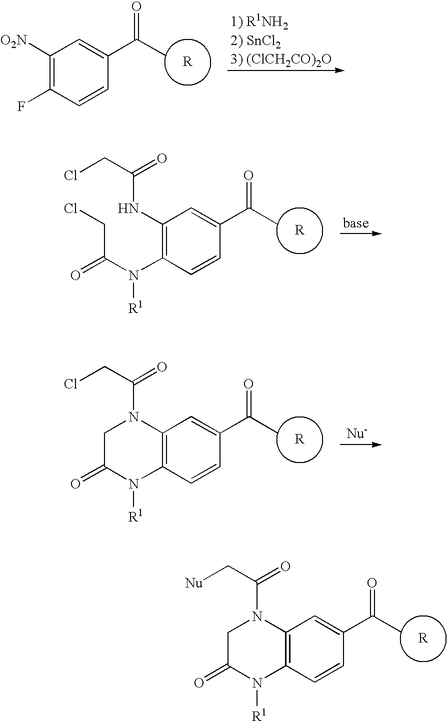

To compare coding efficiency, both noncleavable and cleavable scaffold linker beads were prepared. The resin containing pre-synthesized coding linker was divided into two parts. One part of the resin was treated with TFA to remove the outside Boc protecting groups, and then coupled with Boc-Met-OH, while the other part of the resin remained unmodified. Therefore, both the scaffold linker and the coding linker of the former resin are cleavable, while only the coding linker of the latter resin is cleavable. The inside Fmoc group of the resin is then removed.

A mixture of Trt-Gly-OH, N-phthaloylglycine and 4-nitrophenylacetic acid, whose concentrations have been adjusted according to their relative reactivity (Table 2), are coupled to the interior of the resin. The Trt protecting group on Trt-Gly-OH is removed using 1% TFA, and benzoic acid is coupled to glycine to code the phenylalanine scaffold building block. After Boc deprotection and Boc-Phe-OH coupling, the scaffold is then coupled to the phenylalanine scaffold building block. Propylamine reacts with the N-phthaloylglycine coding functional group to form a stable amide bond (coding building block 3) when replacing the fluoride on the scaffold. Both the nitro scaffold functional group and the nitro coding functional group are reduced with Tin (II) chloride, followed by acylation with chloroacetic anhydride. The tetrahydroquinoxalin is then formed by treating the resin with a base. Finally, the substitution of the remaining chloride with piperidine generates library compound 1 and coding building block 4. A single bead from both parts of the resin is then treated with cyanogen bromide, and analyzed with MALDI-FTMS.

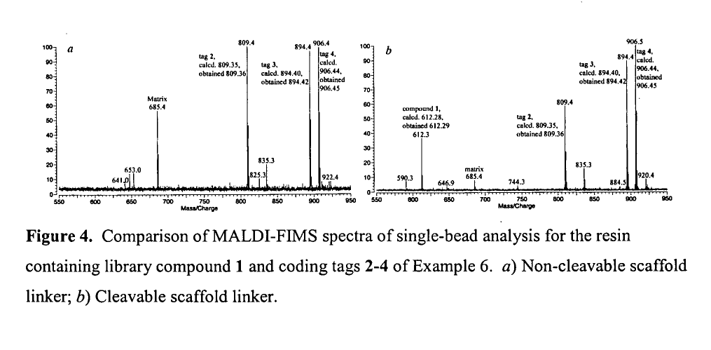

The MS spectra of single-bead analysis are shown in FIG. 4. The results from the cleavable scaffold linker (FIG. 4a) and the non-cleavable scaffold linker (FIG. 4b) beads are consistent. The signals of the three coding tags are clearly detected in both cases, while that of the library compound, as expected, appears only in FIG. 4a.

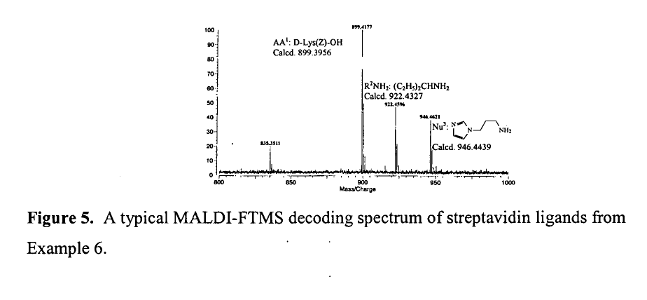

Based on the above preliminary results, a 4-acyl-1,2,3,4-tetrahydroquinoxalin-2-one model library was synthesized (Scheme 35). An amino acid was coupled prior to the scaffold coupling to introduce the first diversity, which was encoded by a pre-coupled acid in the bead interior as shown in Scheme 35. 37 amino acids were used in this step. After scaffold coupling, 51 primary amines were coupled to the scaffold via aromatic nucleophilic substitution. In the last step, 86 nucleophilic reagents including 55 primary amines, 28 secondary amines and 5 thio compounds were used. This results in a library with 37×51×88=166,056 compounds. This library was then screened for ligands that bind to streptavidin followed an established procedure (Liu and Lam 2000, Lam and Lebl 1992). Four positive beads were selected and decoded with MALDI-FTMS. The chemical structures of these streptavidin ligands are shown in Table 5. FIG. 5 shows the MALDI-FTMS spectrum for the coding tags of entry 1 in Table 5.

TABLE 5
Chemical structures of the streptavidin ligands identified from the
4-acyl-1,2,3,4-tetrahydroquinoxalin-2-one library.

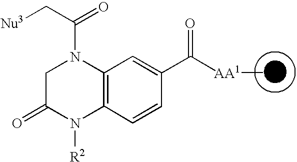

| Entry | AA¹ | R²NH₂ | Nu³ |
|---|---|---|---|
| 1 | 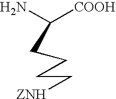 | 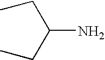 | 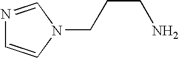 |
| 2 | 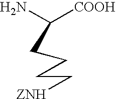 | 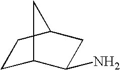 | 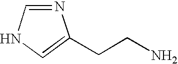 |
| 3 | 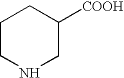 | 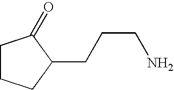 | 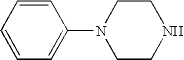 |
| 4 | 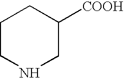 | 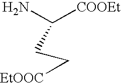 | 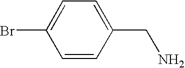 |

### Example 7

### Library Synthesis with a Quinazoline Scaffold using Split-mix Methodology

4-Chloro-7-fluoro-6-nitroquinazoline is used as the scaffold, which is prepared according to the approach described by Barth et al. (Barth et al. 2001). The outer layer of the TentaGel resin beads is first derivatized with Fmoc using the above-mentioned bi-phasic solvent approach. Then, chemical cleavable Boc-linker (same as the linker shown in Scheme 1, but with Boc protecting group) is coupled to the interior of the beads. After cleaving the Boc of the linker with 50% TFA in DCM at room temperature for 30 min, a mixture of coding functional group precursors (4-chloromethylbenzoic acid, 4-nitrophenylacetic acid, and N-Fmoc-nipecotic acid) in a pre-determined ratio based on the relative reactivity (Table 2) are coupled to the linker in the interior of the beads via HOBt/DIC coupling. The Fmoc groups on both the interior (nipecotic acid) and exterior layer are then removed with 20% piperidine in DMF at room temperature (twice, 5 min, 15 min). The bead library is split into different portions to which a specific aldehyde (first reactive component, 10 eq) in trimethyl orthoformate is added. The aldehyde is coupled to the scaffold to form a secondary amine scaffold building block, and to the nipecotic acid coding functional group to form a tertiary amine coding building block simultaneously via reductive alkylation (NaBH₃CN, 1% AcOH, THF). After the reactions are completed, all the beads are combined and mixed, and then added to the scaffold. The secondary amines in the outer layer of the bead first react with the 4-chloro scaffold functional group by nucleophilic substitution, leaving the 7-fluoro group intact (Barth et al. 2001). The beads are split once again; each portion of beads receives a second reactive component (phenols) in the presence of base (e.g. DBU, K₂CO₃). The phenols reacted with the scaffold functional groups and the second coding functional groups (4-chloromethylbenzoic acid) simultaneously. The NO₂ scaffold functional group and the third coding functional group (4-nitrophenylacetic acid) are then reduced with SnCl₂, followed by acylation with the third reactive component: carboxylic acids, anhydrides or acyl chlorides. After the synthesis is complete, the beads are combined and washed thoroughly with organic solvents, water and PBS buffer prior to biological testing.

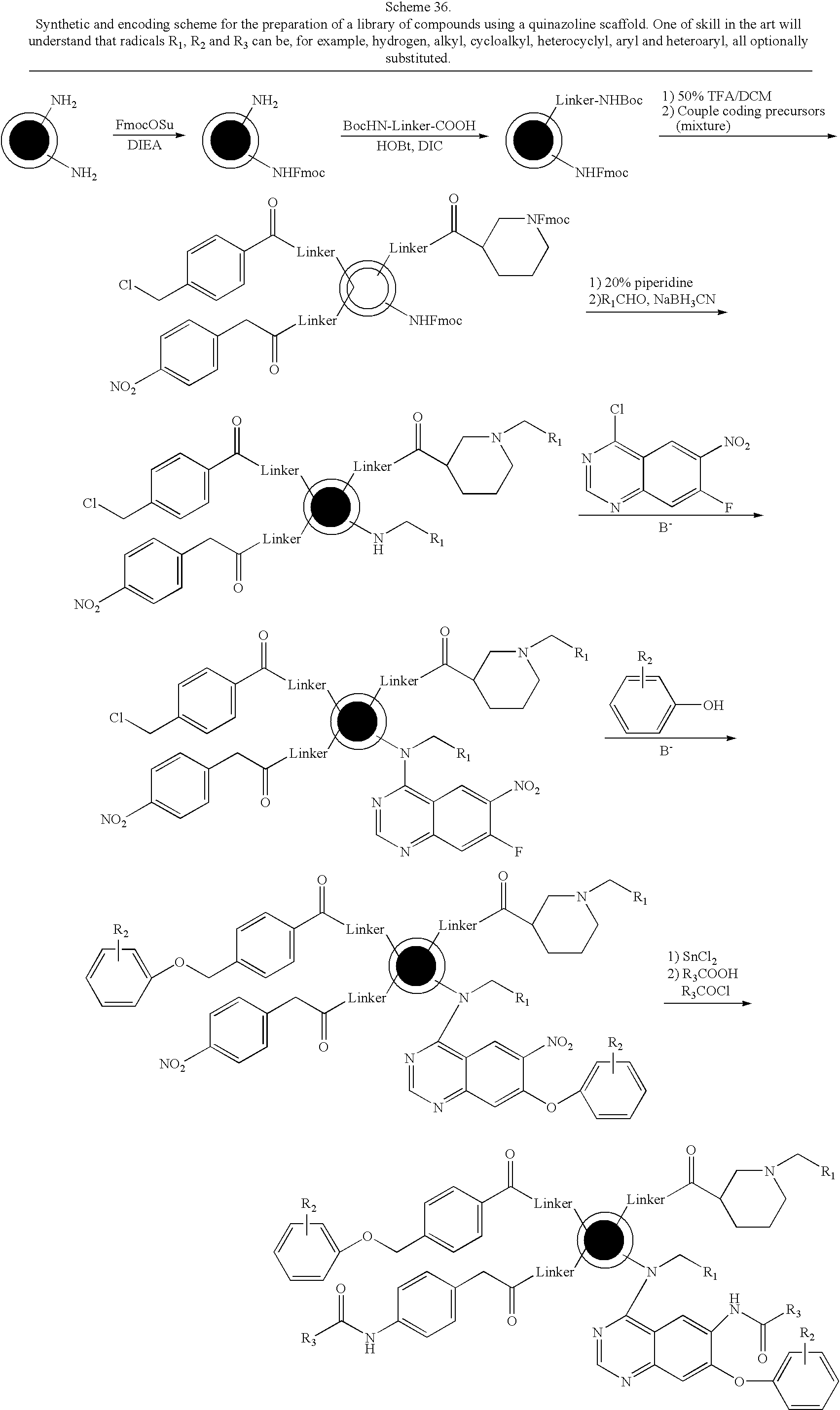

### Example 8

### Library Synthesis with a Quinazoline Scaffold using a Split-mix Methodology

The library synthesis and encoding strategy of library 3 are similar to library 1. Although the scaffold 4,7-di chloro-2-chloromethyl quinazoline for this library is not commercially available, it can be prepared (see Scheme 37) using the similar approach reported by Wright et al. (Wright et al. 2002). The outer layer of the TentaGel resin beads is first derivatized with Alloc using bi-phasic solvent approach. Then, a cleavable linker, i.e. Fmoc-linker (see Scheme 38) is coupled to the interior of the beads. After cleaving the Fmoc of the linker with 20% piperidine in DMF, the mixture of coding functional group precursors (4-chloromethylbenzoic acid, 4-bromoebenzoic acid, and N-Alloc-nipecotic acid) are coupled to the linker in a pre-determined ratio of reaction activity via HOBt/DIC coupling. After removing the Alloc group of both the outer layer and the coding functional group nipecotic acid, with Pd(PPh₃)₄/PhSiH₃ in DCM at room temperature for 30 min (twice), the beads are split into different portions to which each of the first aldehyde reactive components are added (one portion receives one aldehyde). The aldehydes react simultaneously, via reductive alkylation, with the outer layer of the bead to form secondary amines and with the nipecotic acid coding functional group to form tertiary amine coding building blocks. After the reaction is complete, all the beads are combined and mixed, and then added to the scaffold. The 4-chloro group of the scaffold is more reactive than the other two chloro groups, and will react first with the secondary amines in the bead outer layer by nucleophilic substitution (Wright et al. 2002). The beads are then split and each portion of beads receives a second reactive component (aryl boronic acids). The boronic acids are coupled to the scaffold functional group and the second coding functional group (4-bromobenzoic acid) simultaneously via Suzuki reaction. After another round of mix and split, the third reactive component (amines) is coupled with the scaffold functional group and the third coding functional group (chloromethyl benzoic acid) at the same time. In the last step, high temperature or microwave might be required. After the synthesis is complete, the beads are combined and washed thoroughly with organic solvents, water and PBS buffer prior to screening.

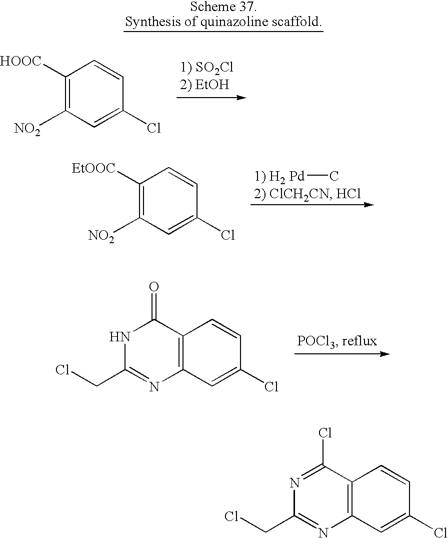

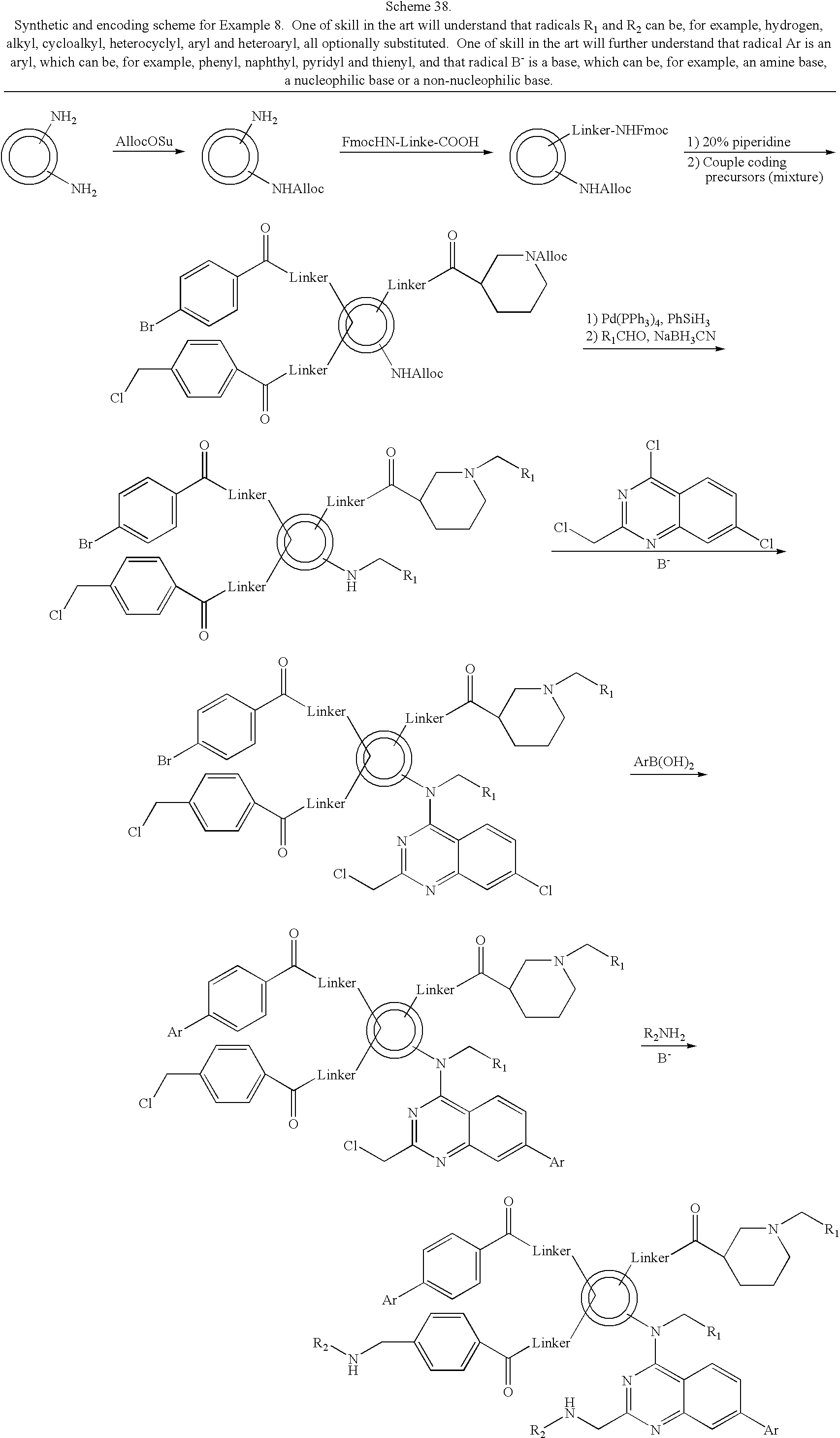

Although the foregoing invention has been described in some detail by way of illustration and example for purposes of clarity of understanding, it will be obvious that certain changes and modifications can be practiced within the scope of the appended claims. In addition, each reference provided herein is incorporated by reference in its entirety to the same extent as if each reference was individually incorporated by reference.

What is claimed is:

1. A method for preparing a library of compounds, comprising:
a) providing a plurality of individual synthesis templates each comprising a solid support, wherein said solid support has an interior portion and an exterior portion each with a plurality of reactive functional groups, wherein said solid support is linked to a scaffold via a scaffold linker, wherein said scaffold has at least two scaffold functional groups, and wherein at least two coding tag precursors, each comprising a coding functional group and a coding linker, are attached to said solid support;
b) contacting a first synthesis template with a first reactive component such that a first scaffold functional group reacts with said first reactive component to afford a first scaffold building block, and a first coding functional group reacts with said first reactive component to afford a first coding building block;
c) contacting said first synthesis template with a successive reactive component such that a subsequent scaffold functional group reacts with said successive reactive component to afford a subsequent scaffold building block, and a subsequent coding functional group reacts with said successive reactive component to afford a subsequent coding building block;
d) repeating step c) until said first compound has been prepared; and
e) subjecting additional synthesis templates to steps b)-d) with additional reactive components to prepare said library of compounds.

2. The method of claim 1, further comprising the following step:
f) cleaving each of said compounds from each of said synthesis templates.

3. The method of claim 1, wherein the number of said scaffold functional groups equals the number of said coding functional groups.

4. The method of claim 1, wherein said reactive component reacts with said scaffold functional group and said coding functional group via a reaction selected from the group consisting of amine acylation, reductive alkylation, aromatic reduction, aromatic acylation, aromatic cyclization, aryl-aryl coupling, [3+2] cycloaddition, Mitsunobu reaction, nucleophilic aromatic substitution, sulfonylation, aromatic halide displacement, Michael addition, Wittig reaction, Knoevenagel condensation, reductive amination, Heck reaction, Stille reaction, Suzuki reaction, Aldol condensation, Claisen condensation, amino acid coupling, amide bond formation, acetal formation, Diels-Alder reaction, [2+2] cycloaddition, enamine formation, esterification, Friedel Crafts reaction, glycosylation, Grignard reaction, Horner-Emmons reaction, hydrolysis, imine formation, metathesis reaction, nucleophilic substitution, oxidation, Pictet-Spengler reaction, Sonogashira reaction, thiazolidine formation, thiourea formation and urea formation.

5. The method of claim 1, wherein said compounds of said library are prepared in parallel.

6. The method of claim 1, wherein at least one of said synthesis templates has a structure of formula I: 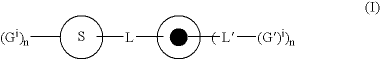
wherein
(Gⁱ)ₙ represents n independent scaffold functional groups, G^{l} to Gⁿ, wherein each Gⁱ is one of said scaffold functional groups; 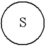
is said scaffold;
L is said scaffold linker; 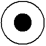
is said solid support, wherein the darkened portion represents said interior portion of said solid support, and the lightened portion represents said exterior portion of said solid support;
(-L′-(G′)ⁱ)ₙ represents n independent coding tag precursors, wherein each of said coding tag precursors comprises one of n independent coding functional groups, (G′)^{l} to (G′)ⁿ, each linked to said solid support via one of n coding linkers, wherein each (G′)ⁱ is one of said coding functional groups, and L′ is said coding linker;
subscript n is an integer from 2 to 10; and
superscript i is an integer from 1 to n.

7. The method of claim 6, wherein said synthesis template has a structure of formula Ia:

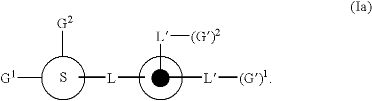

8. The method of claim 6, wherein said synthesis template has a structure of formula Ib:

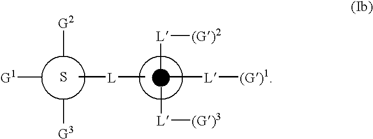

9. The method of claim 1, wherein said synthesis template has a structure of formula Ic: 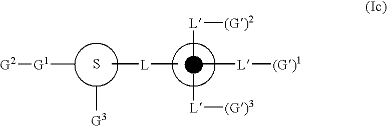
wherein
each of G¹, G² and G³ is one of said scaffold functional groups; 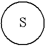
is said scaffold;
L is said scaffold lilnker; 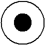
is said scaffold linker;
is said solid support, wherein the darkened portion represents said interior portion of said solid support, and the lightened portion represents said exterior portion of said solid support; and
each of -L′-(G′)¹, -L′-(G′)² and -L′-(G′)³ is one of said coding tag precursors, each comprising said coding functional group linked to said solid support via said coding linker.

10. The method of claim 1, wherein at least one of said synthesis templates has a structure of formula II: 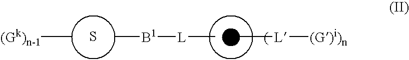
wherein
B^{l} represents a first scaffold building block;
(G^{k})ₙ₋₁ represents n−1 independent scaffold functional groups, G² to Gⁿ, wherein each G^{k} is one of said scaffold functional groups; 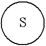
is said scaffold;
L is said scaffold linker; 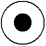
is said solid support, wherein the darkened portion represents said interior portion of said solid support, and the lightened portion represents said exterior portion of said solid support;
(-L′-(G′)ⁱ)ₙ represents n independent coding tag precursors, wherein each of said coding tag precursors comprises one of n independent coding functional groups, (G′)^{l} to (G′)ⁿ, each linked to said solid support via one of n coding linkers, wherein each (G′)ⁱ is one of said coding functional groups, and L′ is said coding linker;
subscript n is an integer from 2 to 10;
superscript i is an integer from 1 to n; and
superscript k is an integer from 2 to n.

11. The method of claim 10, wherein said synthesis template has a structure of formula IIa:

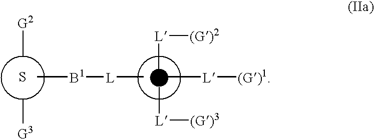

12. The method of claim 1, wherein said steps a)-d) afford a compound template of formula III: 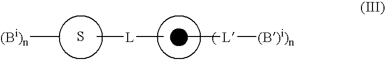
wherein
(Bⁱ)ₙ represents n independent scaffold building blocks, B^{l} to Bⁿ, wherein each Bⁱ is one of said scaffold building blocks; 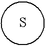
is said scaffold;
L is said scaffold linker; 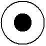
is said solid support, wherein the darkened portion represents said interior portion of said solid support, and the lightened portion represents said exterior portion of said solid support;
(-L′-(B′)ⁱ)ₙ represents n independent coding tags, wherein each of said coding tags comprises one of n independent coding building blocks, (B′)^{l} to (B′)ⁿ, each linked to said solid support via one of n coding linkers, wherein each (B′)^{l} is one of said coding building blocks, and L′ is said coding linker;
subscript n is an integer from 2 to 10; and
superscript i is an integer from 1 to n.

13. The method of claim 1, comprising:
a) providing a plurality of individual synthesis templates according to formula Ib;
b) contacting a first synthesis template with a first reactive component to afford the following structure: 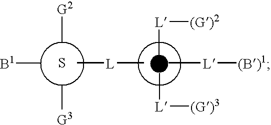
c) contacting said first synthesis template with a successive reactive component to afford the following structure: 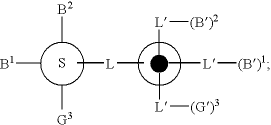
d) repeating step c) to prepare said compound attached to the following compound template according to claim 12: 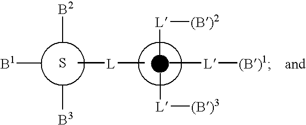
e) subjecting additional of said synthesis templates to steps b)-d) with additional of said reactive components in order to prepare said library of compounds.

14. The method of claim 13, further comprising the following step:
f) cleaving each of said compounds from each of said compound templates.

15. The method of claim 1, wherein, on each of said synthesis templates, each of said scaffold building blocks is encoded by a single coding building block.

16. The method of claim 1, further comprising the following step:
f) decoding each of said compounds by cleaving each of said coding tags from said synthesis template and analyzing said coding tags to determine the identity of said corresponding scaffold building blocks.

17. The method of claim 17, wherein said analyzing is carried out via mass spectrometry.

18. The method of claim 1, wherein said scaffold is the same on each of said synthesis templates.

19. The method of claim 1, wherein at least two different scaffolds are used.

20. The method of claim 1, wherein said scaffold is a member selected from the group consisting of quinazoline, tricyclic quinazoline, purine, pyrimidine, phenylamine-pyrimidine, phthalazine, benzylidene malononitrile, amino acid, tertiary amine, peptide, lactam, sultam, lactone, pyrrole, pyrrolidine, pyrrolinone, oxazole, isoxazole, oxazoline, isoxazoline, oxazolinone, isoxazolinone, thiazole, thiozolidinone, hydantoin, pyrazole, pyrazoline, pyrazolone, imidazole, imidazolidine, imidazolone, triazole, thiadiazole, oxadiazole, benzoffuran, isobenzofuran, dihydrobenzofuran, dihydroisobenzofuran, indole, indoline, benzoxazole, oxindole, indolizine, benzimidazole, benzimidazolone, pyridine, piperidine, piperidinone, pyrimidinone, piperazine, piperazinone, diketopiperazine, metathiazanone, morpholine, thiomorpholine, phenol, dihydropyran, quinoline, isoquinoline, quinolinone, isoquinolinone, quinolone, quinazolinone, quinoxalinone, benzopiperazinone, quinazolinedione, benzazepine and azepine.

21. The method of claim 1, wherein said library of compounds is prepared via a split-mix methodology.

22. A library of compounds prepared by the method of claim 1.

23. A library of compounds prepared by the method of claim 2.

24. A method for identifying a compound of claim 1 that binds to a target, said method comprising:
a) contacting said compound of claim 1 with said target; and
b) determining the functional effect of said compound upon said target.

25. The method of claim 24, wherein said biological target is a protein kinase.

26. A method for identifying a compound of claim 2 that binds to a target, said method comprising:
a) contacting said compound of claim 2 with said target; and
b) determining the functional effect of said compound upon said target.

27. The method of claim 26, wherein said target is a protein kinase.

28. A method for preparing a library of compounds, comprising:
a) providing a population of individual synthesis templates each comprising a solid support, wherein said solid support has an interior portion and an exterior portion each with a plurality of reactive functional groups, wherein said solid support is linked to a scaffold via a scaffold linker, wherein said scaffold has at least two scaffold functional groups, and wherein at least two coding tag precursors, each comprising a coding functional group and a coding linker, are attached to said solid support;
b) splitting said population of synthesis templates into two or more separate pools;
c) contacting said population of synthesis templates with one or more first reactive components in said two or more separate pools such that a first scaffold functional group reacts with one of said first reactive components to afford a first scaffold building block, and a first coding functional group reacts with one of said first reactive components to afford a first coding building block, wherein said contacting step yields subsequent synthesis templates;
d) mixing said subsequent synthesis templates from said two or more separate pools into a single pool;
e) splitting said subsequent synthesis templates into two or more separate pools;
f) contacting said subsequent synthesis templates in said two or more separate pools with a successive reactive component such that a subsequent scaffold functional group reacts with said successive reactive component to afford a subsequent scaffold building block, and a subsequent coding functional group reacts with said successive reactive component to afford a subsequent coding building block, wherein said contacting step yields further synthesis templates;
g) repeating steps d)-f), wherein said further synthesis templates of step f) become said subsequent synthesis templates of step d), until said library of compounds has been prepared.
